(19) United States
(12) Patent Application Publication
(10) Pub. No.: US 2006/0194835 A1
(43) Pub. Date: Aug. 31, 2006

(54) Compositions and methods for treating or preventing flaviviridae infections

(75) Inventors: Dominique Dugourd, Vancouver (CA); Evelina Rubinchik, Richmond (CA); Jacob Clement, North Vancouver (CA); Hillel David Friedland, San Mateo, CA (US)

Correspondence Address: DARBY & DARBY P.C., P. O. BOX 5257, NEW YORK, NY, 10150-5257, US

(73) Assignee: Migenix Inc., Vancouver (CA)

(21) Appl. No.: 11/351,885

(22) Filed: Feb. 9, 2006

Related U.S. Application Data
(60) Provisional application No. 60/651,910, filed on Feb. 9, 2005.
(60) Provisional application No. 60/664,297, filed on Mar. 21, 2005.
(60) Provisional application No. 60/735,464, filed on Nov. 12, 2005.

(51) Int. Cl. A61K 31/4745

(52) U.S. Cl. 514/306

(57) ABSTRACT

The present disclosure relates generally to compositions having a glucosidase inhibitor (castanospermine or a derivative thereof, such as celgosivir) in combination with adjunctive therapies of compounds that alter immune function (such as interferon) and compounds that alter viral replication (such as nucleoside analogues like ribavirin), which can be used to treat or prevent infections caused by or associated with a virus of the Flaviviridae family, particularly infections caused by or associated with Hepatitis C virus (HCV).

### RELATED APPLICATIONS

This application claims the benefit of U.S. Provisional Patent Application No. 60/651,910, filed Feb. 9, 2005, U.S. Provisional Patent Application No. 60/664,297, filed Mar. 21, 2005, and U.S. Provisional Patent Application No. 60/735,464, filed Nov. 12, 2005, which provisional applications are incorporated herein by reference in their entirety.

### TECHNICAL FIELD

The present disclosure relates generally to the treatment of infectious disease, and more specifically, to the use of castanospermine or derivatives thereof in combination with additional anti-viral compounds and/or therapeutic molecules to treat or prevent infections caused by or associated with Flaviviridae, particularly infections caused by or associated with Hepatitis C virus (HCV).

### BACKGROUND

The family Flaviviridae comprises the genera Flavivirus, Pestivirus and Hepacivirus. One significant member of the Flaviviridae family is hepatitis C virus (HCV). HCV was first identified in 1989 and is a major cause of acute hepatitis, responsible for most cases of post-transfusion non-A, non-B hepatitis. HCV is recognized as a major cause of chronic liver disease, including cirrhosis and liver cancer (Hoofnagle, Hepatology 26:15S, 1997). The World Health Organization estimates that close to 170 million people worldwide (i.e., 3% of the world's population) are chronically infected with HCV (Global surveillance and control of hepatitis C. Report of a WHO Consultation organized in collaboration with the Viral Hepatitis Prevention Board, Antwerp, Belgium. J Viral Hepat. 6:35, 1999). In the United States alone, 2.7 million people are chronically infected with HCV with an estimated 8,000 to 10,000 deaths annually (Alter et al., N. Engl J Med. 341:556, 1999). Approximately 3-4 million people are newly infected each year, and 80-85% of these infected patients develop chronic infection with approximately 20-30% of these patients progressing to cirrhosis and end-stage liver disease, frequently complicated by hepatocellular carcinoma (HCC) (see, e.g., Kolykhalov et al., J. Virol. 74:2046, 2000).

Until recently, interferon-α (IFN-α) monotherapy was the only therapy with a proven benefit for the treatment of HCV infection. In the case of genotype 1 HCV infection, only about 50% of patients show an initial response to treatment with IFN-α (i.e., half are non-responders), and the response is not sustainable in the majority of patients. Furthermore, patients suffer considerable side effects due to IFN-α treatment, including flu-like symptoms, malaise, dry skin, depression, leucopenia, thrombocytopenia and thyroid dysfunction. The current standard of care for treating HCV infection is administration of pegylated IFN-α (IFN-α conjugated with polyethylene glycol, PEG) with the broad spectrum nucleoside analogue ribavirin. Unfortunately, treatment with IFN-α or IFN-α with ribavirin is not particularly effective if a person: is infected with genotype 1 HCV (the most common genotype in the U.S. and Europe), has a high HCV viral load (greater than two million copies), has been infected with HCV for a longer time, has moderate to severe disease, is male, and is older.

Other drugs are being tested for combination therapy with interferon-α, such as histamine dihydrochloride, and a synthetic version of thymosin-α-1, a hormone that stimulates T-cells and natural killer cells. Amantadine, an antiviral medication used to treat influenza A, has been studied in combination with interferon and ribavirin. Unfortunately, amantadine shows some significant side-effects and the combination studies conducted to date have been disappointing (see, e.g., Khalili et al., Am. J. Gastroenterol. 98:1284-9, 2000; Brillanti et al., Ital. J. Gastroenterol. Hepatol. 31:130, 1999). HCV helicase inhibitors, HCV protease inhibitors (including a serine protease inhibitor), and RNA-dependent RNA HCV genome polymerase inhibitors that would potentially block HCV viral replication are also currently under study. Overall, HCV, and especially genotype 1, is a difficult disease to manage due to the lack of good conventional treatment options.

Hence, a need exists for identifying and developing anti-Flaviviridae therapies with improved anti-viral activity and reduced toxicity as compared to current treatment regimes (such as those used for the treatment of HCV). The present invention meets such needs, and further provides other related advantages.

### SUMMARY

The present invention generally provides compositions comprising a combination of a glucosidase inhibitor, and other anti-Flaviviridae compounds, such as agents that alter immune function or agents that alter Flaviviridae functions. Exemplary glucosidase inhibitors include castanospermine or derivatives thereof, such as celgosivir; agents that alters immune function include interferons; and agents that alters replication of Flaviviridae include nucleoside inhibitors such as ribavirin or 2′-C-methyl cytidine (NM-1 07). Such combinations of compounds, or compositions thereof, are useful for treating or preventing, for example, Flaviviridae viral infections such as those caused by hepatitis C virus (HCV). In particular, the present disclosure provides castanospermine or derivatives thereof (such as celgosivir) in combination with two other anti-Flaviviridae compounds, providing unexpectedly high or synergistic inhibitory activity against HCV, and an unexpected decrease in the cytotoxicity of known anti-Flaviviridae compounds (such as interferon and ribavirin).

In one aspect, the instant disclosure provides a composition comprising a glucosidase inhibitor, an agent that alters immune function, and an agent that alters replication of Flaviviridae. In certain embodiments, the glucosidase inhibitor has the following structural formula (I):

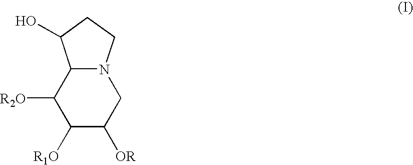

wherein R, R₁ and R₂ are independently hydrogen, C₁₋₁₄ alkanoyl, C₂₋₁₄ alkenoyl, cyclohexanecarbonyl, C₁₋₈ alkoxyacetyl,

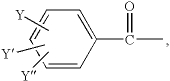

naphthalenecarbonyl optionally substituted by methyl or halogen; phenyl(C₂₋₆ alkanoyl) wherein the phenyl is optionally substituted by methyl or halogen; cinnamoyl; pyridinecarbonyl optionally substituted by methyl or halogen; dihydropyridine carbonyl optionally substituted by C₁₋₁₀ alkyl; thiophenecarbonyl optionally substituted by methyl or halogen; or furancarbonyl optionally substituted by methyl or halogen; Y is hydrogen, C₁₋₄ alkyl, C₁₋₄ alkoxy, halogen, trifluoromethyl, C₁₋₄ alkylsulphonyl, C₁₋₄ alkylmercapto, cyano or dimethylamino; Y′ is hydrogen, C₁₋₄ alkyl, C₁₋₄ alkoxy, halogen or it is combined with Y to give 3,4-methylenedioxy; Y″ is hydrogen, C₁₋₄ alkyl, C₁₋₄ alkoxy or halogen; and pharmaceutically acceptable salts thereof. In another embodiment, the glucosidase inhibitor has the structural formula described above with R, R₁ and R₂ being selected in such a way that at least one of them, but not more than two of them, is hydrogen; or a pharmaceutically acceptable salt or derivative thereof. In related embodiments, the glucosidase inhibitor can be (a) [1S-(1α,6β,7α,8β,8aβ)]-octahydro-1,6,7,8-indolizinetetrol 6-benzoate; (b) [1S-(1α,6β,7α,8β,8aβ)]-octahydro-1,6,7,8-indolizinetetrol 7-benzoate; (c) [1S-(1α,6β,7α,8β,8aβ)]-octahydro-1,6,7,8-indolizinetetrol 6-(4-methylbenzoate); (d) [1S-(1α,6β,7α,8β,8aβ)]-octahydro-1,6,7,8-indolizinetetrol 7-(4bromobenzoate); (e) [1S-(1α,6β,7α,8β,8aβ)]-octahydro-1,6,7,8-indolizinetetrol 6,8-dibutanoate; (f) [1S-(1β,6β,7α,8β,8aβ)]-octahydro-1,6,7,8-indolizinetetrol 6-butanoate; (g) [1S-(1α,6β,7α,8β,8aβ)]-octahydro-1,6,7,8-indolizinetetrol 6-(2-furancarbonxylate); (h) [1S-(1α,6β,7α,8β,8aβ)]-octahydro-1,6,7,8-indolizinetetrol 7-(2,4-dichlorobenzoate); (i) [1S-(1α,6β,7α,8β,8aβ)]-octahydro-1,6,7,8-indolizinetetrol 6-(3-hexenoate); (j) [1S-(1α,6β,7α,8β,8aβ)]-octahydro-1,6,7,8-indolizinetetrol 6-octanoate; (k) [1S-(1α,6β,7α,8β,8aβ)]-octahydro-1,6,7,8-indolizinetetrol 6-pentanoate; (l) an O-pivaloyl ester; (m) a 2-ethyl-butyryl ester; (n) a 3,3-dimethylbutyryl ester; (o) a cyclopropanoyl ester; (p) a 4-methoxybenzoate ester; (q) a 2-aminobenzoate ester; (r) castanospermine or (s) a mixture of at least two of (a)-(r). In still other embodiments, the agent that alters immune function can be an interferon, such as interferon-α or pegylated interferon-α. In further embodiments, the agent that alters viral replication can be a nucleoside analogue, such as ribavirin.

### BRIEF DESCRIPTION OF THE DRAWINGS

FIGS. 1A and 1B illustrate the 3-D and 2-D view, respectively, of the double combination synergy volume of castanospermine and IFN-α.

FIG. 2 is an isobologram of the double combination of castanospermine and IFN-α.

FIGS. 3A and 3B illustrate the 3-D and 2-D view, respectively, of the double combination synergy volume of celgosivir and IFN-α.

FIG. 4 is an isobologram of the double combination of celgosivir and IFN-α.

FIGS. 5A and 5B illustrate the 3-D and 2-D view, respectively, of the double combination synergy volume of castanospermine and ribavirin.

FIG. 6 is an isobologram of the double combination of castanospermine and ribavirin.

FIGS. 7A and 7B illustrate the 3-D and 2-D view, respectively, of the double combination synergy volume of celgosivir and ribavirin.

FIG. 8 is an isobologram of the double combination of celgosivir and ribavirin.

FIGS. 9A and 9B illustrate the 3-D and 2-D view, respectively, of the double combination synergy volume of castanospermine and NM107.

FIG. 10 is an isobologram of the double combination of castanospermine and NM107.

FIGS. 11A and 11B illustrate the 3-D and 2-D view, respectively, of the double combination synergy volume of celgosivir and NM107.

FIG. 12 is an isobologram of the double combination of celgosivir and NM107.

FIGS. 13A and 13B illustrate the 3-D and 2-D view, respectively, of the double combination synergy volume of IFN-α and ribavirin.

FIG. 14 is an isobologram of the double combination of IFN-α and ribavirin.

FIGS. 15A and 15B illustrate an Fa-CI graph and isolbologram, respectively, of the double combination of castanospermine and Peg-IFN-α2b.

FIGS. 16A and 16B illustrate an Fa-CI graph and isolbologram, respectively, of the double combination of celgosivir and Peg-IFN-α2b.

FIGS. 17A and 17B illustrate an Fa-CI graph and isolbologram, respectively, of the double combination of celgosivir and IFN-αcon-1.

FIGS. 18A and 18B illustrate an Fa-CI graph and isolbologram, respectively, of the double combination of celgosivir and IFN-α-n3.

FIGS. 19A-19F illustrate the 3-D and 2-D view, respectively, of the combination synergy volume of celgosivir and IFN-α with varying concentrations of ribavirin.

FIGS. 20A-20F illustrate the 3-D and 2-D view, respectively, of the combination synergy volume of castanospermine and IFN-α with varying concentrations of ribavirin.

FIG. 21 illustrates an Fa-CI graph of the triple combination of celgosivir, IFN-λ1 and NM107.

FIGS. 22A and 22B illustrates an Fa-CI graph of the double combination of celgosivir and ribavirin and the triple combination of celgosivir, ribavirin and IFN-α2b, respectively.

FIGS. 23A and 23B illustrate the 3-D and 2-D view, respectively, of the combination antagonism volume of celgosivir and IFN-α.

FIGS. 24A and 24B illustrate the 3-D and 2-D view, respectively, of the combination antagonism volume of celgosivir and ribavirin.

FIGS. 25A and 25B illustrate the 3-D and 2-D view, respectively, of the combination antagonism volume of castanospermine and IFN-α.

FIGS. 26A and 26B illustrate the 3-D and 2-D view, respectively, of the combination antagonism volume of castanospermine and ribavirin.

FIG. 27 illustrates the synergy data in a linear graph.

FIGS. 28A-28C graphically illustrate the effect of anti-diarrheal agents on the pharmacokinetics (PK) of orally administered celgosivir. The graphs show the plasma concentration of castanospermine versus time plots for various groups of rats, as indicated.

### DETAILED DESCRIPTION

The present disclosure provides compositions and methods for using castanospermine or derivatives thereof (such as celgosivir) in combination with other anti-viral compounds to treat or prevent infectious diseases. In particular, these compositions are useful for treating or preventing viral infections, such as hepatitis C virus (HCV) infections. The invention, therefore, relates generally to the surprising discovery that castanospermine or derivatives thereof (e.g., ester derivatives) administered in combination with other therapeutic compounds, such as interferon-alpha (IFN-α, interferon-α, alpha-interferon, or α-interferon) or ribavirin, have an unexpectedly high activity against Flaviviridae, such as HCV. Also, the double combination of castanospermine with IFN-α or castanospermine with ribavirin results in a surprising reduction in the cytotoxicity of IFN-α and ribavirin, respectively. In addition, these combination therapies can be combined with other therapeutic adjuncts that reduce or alleviate associated side effects, such as anti-diarrheal agents. Accordingly, the compositions of the instant disclosure are useful, for example, in the treatment of HCV infections and HCV-related disease. In addition, the compounds and compositions provided herein are useful as research tools for in vitro and cell-based assays to study the biological mechanisms of, for example, HCV infection (e.g., replication and transmission).

By way of background, glycoproteins are classified into two major classes according to the linkage between sugar and amino acid of a protein. The most common is an N-glycosidic linkage between an asparagine of a protein and an N-acetyl-D-glucosamine residue of an oligosaccharide. N-linked oligosaccharides, following attachment to a polypeptide backbone, are processed by a series of specific enzymes in the endoplasmic reticulum (ER), and this processing pathway has been well characterized.

In the ER, α-glucosidase I is responsible for the removal of the terminal α-1,2 glucose residue from the precursor oligosaccharide, and α-glucosidase II removes the two remaining α-1,3 linked glucose residues prior to removal of mannose residues by mannosidases and further processing reactions involving various transferases. These oligosaccharide “trimming” reactions enable glycoproteins to fold correctly and to interact with chaperone proteins such as calnexin and calreticulin for transport through the Golgi apparatus. Inhibitors of key enzymes in this biosynthetic pathway, particularly those blocking α-glucosidases and α-mannosidases, prevent replication of several enveloped viruses. Such inhibitors may act by interfering with the folding of the viral envelope glycoprotein, thus preventing the initial virus-host cell interaction or a subsequent fusion. These inhibitors may also prevent viral duplication by preventing the construction of the proper glycoprotein required for the completion of the viral membrane.

For example, nonspecific glycosylation inhibitors 2-deoxy-D-glucose and β-hydroxy-norvaline inhibited expression of HIV glycoproteins and blocked the formation of syncytia (Blough et al., Biochem. Biophys. Res. Commun. 141:33, 1986). Viral multiplication of HIV-infected cells treated with these agents is stopped, presumably because of the unavailability of glycoprotein required for viral membrane formation. The glycosylation inhibitor 2-deoxy-2-fluoro-D-mannose exhibited antiviral activity against influenza-infected cells by preventing the glycosylation of viral membrane protein (McDowell et al., Biochemistry, 24:8145, 1985). Lu et al. presented evidence that N-linked glycosylation was necessary for hepatitis B virus secretion (Virology 213: 660, 1995), and Block et al. showed that secretion of human hepatitis B virus was inhibited by the imino sugar N-butyldeoxynojirimycin (Proc. Natl. Acad. Sci. USA 91: 2235, 1994; see also, e.g., WO9929321).

In the present description, any concentration range, percentage range, integer range or ratio range is to be understood to include the value of any integer within the recited range and, when appropriate, fractions thereof (such as one tenth and one hundredth of an integer), unless otherwise indicated. As used herein, “about” or “comprising essentially of” mean ±15%. The use of the alternative (e.g., “or”) should be understood to mean either one, both, or any combination thereof of the alternatives. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present invention.

As used herein, the term “alkyl” refers to a saturated or unsaturated, branched, straight-chain or cyclic monovalent hydrocarbon group derived by the removal of one hydrogen atom from a single carbon atom of a parent alkane, alkene or alkyne. Alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1yn-3-yl, but-3-yn-1-yl, etc.; and the like.

The term “alkyl” is specifically intended to include straight- or branched-hydrocarbons having from 1 to 25 carbon atoms, or 5 to 20, or 10 to 18, or 1 to 5. The alkyls may have any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. When a specific level of saturation is intended, the expressions “alkanyl,” “alkenyl,” and “alkynyl” are used. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. The alkenyl group may be substituted or unsubstituted.

“Alkynyl” refers to an unsaturated branched, straight chain or cyclic alkyl group having at least one carbon-carbon triple bond derived by the removal of one hydrogen atom from a single carbon atom of a parent alkyne. Alkynyl groups can include ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. When a specific level of saturation is intended, the nomenclature alkanyldiyl, alkenyldiyl or alkynyldiyl is used. In certain embodiments, the alkyldiyl group is (C₁-C₄) alkyldiyl. Other embodiments may include saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” refers to a straight-chain alkyldiyl group having two terminal monovalent radical centers derived by the removal of one hydrogen atom from each of the two terminal carbon atoms of straight-chain parent alkane, alkene or alkyne. Alkyleno groups include methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc.; and the like. When a specific level of saturation is intended, the nomenclature alkano, alkeno or alkyno is used. In certain embodiments, the alkyleno group is (C₁-C₆) or (C₁-C₄) alkyleno. Other embodiments may include straight-chain saturated alkano groups, e.g., methano, ethano, propano, butano, and the like.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatoms or heteroatomic groups. Heteroatoms or heteroatomic groups that can be included in these groups include —O—, —S—, —Se—, —O—O—, —S—S—, —O—S—, —O—S—O—, —O—NR′—, —NR′—, —NR′—NR′—, ═N—N═, —N═N—, —N═N—NR′—, —PH—, —P(O)₂—, —O—P(O)₂—, —SH₂—, —S(O)₂—, —SnH₂— and the like, and combinations thereof, including —NR′—S(O)₂—, wherein each R′ is independently selected from hydrogen, alkyl, alkanyl, alkenyl, alkynyl, aryl, arylalkyl, heteroaryl and heteroarylalkyl, as defined herein.

“Aryl” refers to a monovalent aromatic hydrocarbon group derived by the removal of one hydrogen atom from a single carbon atom of a parent aromatic ring system. Aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, the aryl group can be (C₅-C₁₄) aryl, or more specifically can be (C₅-C₁₀). Some embodiments may include aryls that are cyclopentadienyl, phenyl and naphthyl. The aryl group may be substituted or unsubstituted.

“Arylalkyl” refers to an acyclic alkyl group in which one of the hydrogen atoms bonded to a carbon atom, typically a terminal or sp³ carbon atom, is replaced with an aryl group. Arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. When specific alkyl moieties are intended, the nomenclature arylalkanyl, arylakenyl or arylalkynyl is used. In certain embodiments, the arylalkyl group may be (C₆-C₂₀) arylalkyl, e.g., the alkanyl, alkenyl or alkynyl moiety of the arylalkyl group is (C₁-C₆) and the aryl moiety is (C₅-C₁₄). In other embodiments the arylalkyl group may be (C₆-C₁₃), e.g., the alkanyl, alkenyl or alkynyl moiety of the arylalkyl group is (C₁-C₃) and the aryl moiety is (C₅-C₁₀).

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-14 membered heteroaryl, or a 5-10 membered heteroaryl. Other embodiments may include heteroaryl groups that have been derived from thiophene, pyrrole, benzothiophene, benzofuran, indole, pyridine, quinoline, imidazole, oxazole and pyrazine. The heteroaryl group may be substituted or unsubstituted.

“Heteroalicyclic” refers to a monocyclic or fused ring group having in the ring(s) one or more atoms selected from, for example, nitrogen, oxygen and sulfur. The rings may also have one or more double bonds. However, the rings do not necessarily have a completely conjugated π-electron system. The heteroalicyclic ring may be substituted or unsubstituted. When substituted, the substituted group(s) may be selected independently from alkyl, aryl, haloalkyl, halo, hydroxy, alkoxy, mercapto, cyano, sulfonamidyl, aminosulfonyl, acyl, acyloxy, nitro, and substituted amino.

“Heteroarylalkyl” refers to an acyclic alkyl group in which one of the hydrogen atoms bonded to a carbon atom, such as a terminal or sp³ carbon atom, is replaced with a heteroaryl group. When one or more specific alkyl moiety is intended, the nomenclature heteroarylalkanyl, heteroarylakenyl or heterorylalkynyl is used. In certain embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety may be a 5-14-membered heteroaryl. In other embodiments, the heteroarylalkyl may be a 6-13 membered heteroarylalkyl, e.g., the alkanyl, alkenyl or alkynyl moiety is 1-3 membered and the heteroaryl moiety is a 5-10 membered heteroaryl.

The various naphthalenecarbonyl, pyridinecarbonyl, thiophenecarbonyl and farancarbonyl groups referred to herein include the various position isomers and these can be naphthalene-1-carbonyl, naphthalene-2-carbonyl, nicotinoyl, isonicotinoyl, N-methyl-dihydro-pyridine-3-carbonyl, thiophene-2-carbonyl, thiophene-3-carbonyl, furan-2-carbonyl and furan-3-carbonyl. The naphthalene, pyridine, thiophene and furan groups can be optionally further substituted, as indicated herein.

“Halogen” or “halo” refers to fluoro (F), chloro (Cl), bromo (Br), iodo (I). As used herein, —X refers to independently any halogen.

“Acyl” group refers to the C(O)—R″ group, where R″ can be selected from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Acyl groups include aldehydes, ketones, acids, acid halides, esters and amides. Certain exemplary acyl groups can be carboxy groups, e.g., acids and esters. Esters include amino acid ester derivatives. The acyl group may be attached to a compound's backbone at either end of the acyl group, i.e., via the C or the R″. When the acyl group is attached via the R″, then C can bear another substituent, such as hydrogen, alkyl, and the like.

“Substituted” refers to a group in which one or more hydrogen atoms are each independently replaced with the same or different substituent(s). Substituents may include —X, —R¹³, —O—, ═O, —OR, —SR¹³, —S—, ═S, —NR¹³R¹³, ═NR¹³, CX₃, —CF₃, —CN, —OCN, —SCN, —NO, NO₂, ═N₂, —N₃, —S(O)₂O—, —S(O)₂OH, —S(O)₂R¹³, —OS(O₂)O—, —OS(O)₂R¹³, —P(O)(O)⁻)₂, —P(O)(OH)(O⁻), —OP(O)₂(O⁻), —C(O)R¹³, —C(S)R¹³, —C(O)OR¹³, —C(O)O⁻, —C(S)OR¹³, and —C(N¹³)NR¹³R¹³, wherein each X is independently a halogen; each R¹³ may independently be hydrogen, halogen, alkyl, aryl, arylalkyl, arylaryl, arylheteroalkyl, heteroaryl, heteroarylalkyl NR¹⁴R¹⁴, —C(O)R¹⁴, and —S(O)₂R¹⁴; and each R14 may independently be hydrogen, alkyl, alkanyl, alkynyl, aryl, arylalkyl, arylheteralkyl, arylaryl, heteroaryl or heteroarylalkyl.

“Prodrug” herein refers to a compound that is converted into the parent compound or a metabolite thereof in vivo. Prodrugs often are useful because, in some situations, they may be easier to administer than the parent compound. For example, the prodrug may be more bioavailable by oral administration or for cellular uptake than a parent compound. The prodrug may also have improved solubility in pharmaceutical compositions over the parent compound or an extended half-life in vivo. An example of a prodrug can be a compound as described herein that is administered as an ester (a “prodrug”) to, for example, facilitate transmittal across a cell membrane (when water solubility is detrimental to mobility across such as membrane). In certain embodiments, a prodrug compound may be inactive (or less active) until converted into the parent compound, a metabolite, or a further activated metabolite thereof.

“Pharmaceutically acceptable salt” refers to a salt of a compound of the invention that is pharmaceutically acceptable and that possesses the desired pharmacological (e.g., anti-viral) activity. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

Castanospermine and Derivatives Thereof

As set forth above, the present invention provides a glucosidase inhibitor, such as castanospermine or derivatives thereof and pharmaceutically acceptable salts thereof, and compositions of such compounds for use in combination therapies. For example, compositions disclosed herein comprise a glucosidase inhibitor (e.g., castanospermine or a derivative thereof) in combination with an inhibitor of viral replication (e.g., ribavirin or 2′-C-methyl cytidine or valopicitabine) and a compound that alters immune function or response (e.g., interferon or pegylated interferon), which combinations have unexpectedly high anti-viral activity, and in particular, high anti-HCV activity, as well as a reduction in cytotoxicity of the viral replication inhibitor and agent that alters immune function. In addition, such compositions may optionally be combined with other adjunctive therapeutics, such as anti-diarrheal agents.

Exemplary glucosidase inhibitors include castanospermine and certain imino sugars, such as deoxynojirimycin (DNJ), which are ER α-glucosidase inhibitors that potently inhibit the early stages of glycoprotein processing (see, e.g, Ruprecht et al., J. Acquir. Immune Defic. Syndr. 2:149, 1989; see also, e.g., Whitby et al., Antiviral Chem. Chemother. 15:141, 2004; Branza-Nichita et al., J. Virol. 75:3527, 2001; Courageot et al., J. Virol. 75:564, 2000; Choukhi et al., J. Virol. 72:3851, 1998; WO 99/29321; WO 02/089780). However, the effects of the inhibitors differ substantially depending on the system to which they are applied, and may exhibit quite different specificities—castanospermine apparently being relatively specific for α-glucosidase I.

Castanospermine is a natural alkaloid derived from the black bean or Moreton chestnut tree (Castanospermum australe) (Hohenschutz et al., Phytochemistry 20:811-14 (1981)). Castanospermine is water soluble and, thus, is readily isolated according to procedures practiced in the art (see, e.g., Alexis Platform, San Diego, Calif.). The highest concentration of the compound is found in seeds and seed pods (Pan et al., Arch. Biochem. Biophys. 303:134, 1993). In addition to inhibiting the enzymatic activity of α-glucosidase I, castanospermine also inhibits intestinal glycosidases, such as maltase and sucrase, which may result in gastrointestinal side effects, such as gas, flatulence or diarrhea (Saul et al., Proc. Natl. Acad. Sci. USA 82:93, 1985). Such side effects may be reduced, minimized or prevented in a subject receiving castanospermine by altering the subject's diet to a starch-free, high-glucose diet (see, e.g., Saul et al., supra). Alternatively, as provided herein, castanospermine or derivatives thereof may be optionally combined with an adjunctive therapy that reduces such gastrointestinal side-effects, such as an anti-diarrheal agent.

Castanospermine has the following formula, 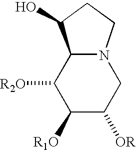
wherein R, R₁, and R₂ are hydrogen. Systematically, this compound can be named in several ways: [1S-(1α,6β,7α,8β,8aβ)]-octahydro-1,6,7,8-indolizinetetrol or [1S,(1S,6S,7R,8R,8aR)-1,6,7,8-tetrahydroxyindolizidine or 1,2,4,8-tetradeoxy-1,4,8-nitrilo-L-glycero-D-galacto-octitol. The term castanospermine or the first systematic name will be used herein.

The castanospermine esters of the present disclosure may be prepared by the reaction of castanospermine with an appropriate acid chloride or anhydride in an inert solvent (see, e.g., U.S. Pat. Nos. 4,970,317; 5,017,563; 5,959,111). The halide can be a chloride or bromide, and the anhydride can include mixed anhydrides. The relative amount of the acid halide or anhydride used, the relative amount of solvent, the temperature and the reaction time are all controlled so as to minimize the number of hydroxyl groups that will be acylated. Thus, only a limited excess of an acid derivative may be used, which means up to about a three-fold excess of an acylating agent.

Use of a solvent in relatively large amounts serves to dilute the reactants and suppress the amount of higher acylated products that form. In certain embodiments, a solvent is used that can dissolve the reactants without reacting with them.

In certain embodiments, it may be advantageous to carry out the reaction in the presence of a tertiary amine, which can react with and remove acid formed during the course of the reaction. The tertiary amine can be added to the mixture, or it can itself be used in excess and serve as the solvent. For example, pyridine can be used. As indicated herein, the time and the temperature may likewise be controlled to limit the amount of acylation that takes place. In some embodiments, the reaction may be carried out with cooling in an ice-bath for a period of about 16 hours to give generally monoesters, or the reaction time may be extended to a longer period, such as 7 days, if more diesters are desired. The reaction can actually be carried out at higher temperatures, and heating can be used as long as the various factors involved are properly controlled.

When the reaction is carried out as described herein, the final reaction mixture may still contain a considerable amount of unreacted castanospermine. This unreacted material can be recovered from the reaction mixture and recycled in subsequent reactions and, therefore, increase the overall amount of castanospermine converted to an ester. This recycling is particularly useful when the reaction is carried out under conditions that would favor the isolation of monoesters. The procedures, as described herein, can generally yield 6- or 7-monoesters, or 6,7- or 6,8-diesters. Other isomers can be obtained by appropriate use of blocking groups. For example, castanospermine can be reacted with 2-(dibromomethyl)benzoyl chloride to give the 6,7-diester. This diester is then reacted with an appropriate acid halide or anhydride to give the corresponding 8-ester. The two protecting groups are then readily removed by conversion of the two dibromomethyl groups to formyl (using silver perchlorate and 2,4,6-collidine in aqueous acetone) followed by hydrolysis of the formylbenzoic acid ester obtained using morpholine and hydroxide ion. The indicated procedure can be used in a similar way to give diester isomers.

With 1,8-O-isopropylidenecastanospermine or 1,8-cyclohexylidene castanospermine, the reaction with an acid chloride in a standard esterification procedure favors the formation of the 6-ester almost exclusively. The isopropylidene or cyclohexylidene group may then be removed by treatment with an acid, such as 4-toluene sulphonic acid. The starting ketal compounds are themselves obtained from castanospermine 6,7-dibenzoate. This dibenzoate may then be reacted with 2-methoxypropene or 1-methoxycyclohexene and acid to introduce the 1,8-O-isopropylidene or 1,8-O-cyclohexylidene group, and the two benzoate ester groups are removed by hydrolysis with base, such as sodium hydroxide, or by transesterification with sodium or potassium alkoxide as the catalyst.

In certain embodiments, the present disclosure provides compositions and methods for treating or preventing a Flaviviridae infection, comprising administering to a subject a composition. Compositions of the instant disclosure include a glucosidase inhibitor, a viral replication inhibitor and an agent that alters immune function, wherein the glucosidase inhibitor has the following structural formula (I):

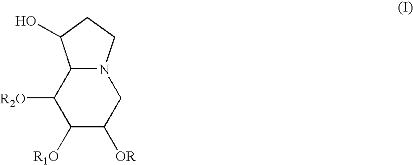

wherein R, R₁ and R₂ are independently hydrogen, C₁₋₁₄ alkanoyl, C₂₋₁₄ alkenoyl, cyclohexanecarbonyl, C₁₋₈ alkoxyacetyl,

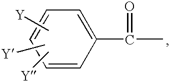

naphthalenecarbonyl optionally substituted by methyl or halogen; phenyl(C₂₋₆ alkanoyl) wherein the phenyl is optionally substituted by methyl or halogen; cinnamoyl; pyridinecarbonyl optionally substituted by methyl or halogen; dihydropyridine carbonyl optionally substituted by C₁₋₁₀ alkyl; thiophenecarbonyl optionally substituted by methyl or halogen; or furancarbonyl optionally substituted by methyl or halogen; Y is hydrogen, C₁₋₄ alkyl, C₁₋₄ alkoxy, halogen, trifluoromethyl, C₁₋₄ alkylsulphonyl, C₁₋₄ alkylmercapto, cyano or dimethylamino; Y′ is hydrogen, C₁₋₄ alkyl, C₁₋₄ alkoxy, halogen or it is combined with Y to give 3,4-methylenedioxy; Y″ is hydrogen, C₁₋₄ alkyl, C₁₋₄ alkoxy or halogen; or a pharmaceutically acceptable salt or derivative thereof. In another embodiment, the glucosidase inhibitor structural formula (I) has the following stereochemistry:

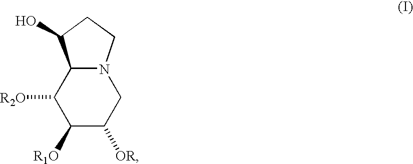

In certain embodiments, the glucosidase inhibitor structural formula (I) as described herein has R, R₁ and R₂ selected in such a way that at least one of them, but not more than two of them, is hydrogen. In still other embodiments, a castanospermine ester has a structure as shown in Table 1.

TABLE 1

| | Structure | | Structure |
|---|---|---|---|
| Compound | R | Compound | R |
| CAST | H | MDL 29270 | H |
| MDL 28574 | CH₃(CH₂)₂—CO— | MDL 44370 | 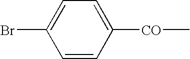 |
| MDL 43305 | 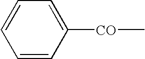 | MDL 29797 | CH₃(CH₂)₆—CO— |
| MDL 28653 | 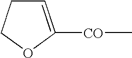 | MDL 29710 | CH₃(CH₂)₃—CO— |
| MDL 29435 | 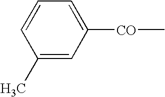 | MDL 29513 | CH₃CH₂(CH₂)₂CH₂—CO— |
| MDL 29204 | 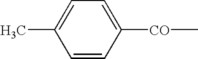 | | |

*In MDL 29270, R₁ is 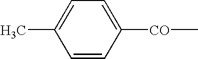 ; in all other structures R₁ is H

In certain embodiments, provided are castanospermine esters of structure (I) wherein R₁ may be a C₁₋₈ alkanoyl, C₂₋₁₀ alkenoyl, C₁₋₈ alkoxy-acetyl, or a benzoyl optionally substituted with an alkyl or halogen group. In still other embodiments, R₁ may be a C₁₋₈ alkanoyl, C₂₋₈ alkenoyl, C₁₋₈ alkoxyacetyl, or a benzoyl optionally substituted with a methyl, bromo, chloro, or fluoro group.

In still further embodiments, the glucosidase inhibitor may be (a) [1S-(1α,6β,7α,8β,8aβ)]-octahydro-1,6,7,8-indolizinetetrol 6-benzoate; (b) [1S-(1α,6β,7α,8β,8aβ)]-octahydro-1,6,7,8-indolizinetetrol 7-benzoate; (c) [1S-(1α,6β,7α,8β,8aβ)]-octahydro-1,6,7,8-indolizinetetrol 6-(4-methylbenzoate); (d) [1S-(1α,6β,7α,8β,8aβ)]-octahydro-1,6,7,8-indolizinetetrol 7-(4bromobenzoate); (e) [1S-(1α,6β,7α,8β,8aβ)]-octahydro-1,6,7,8-indolizinetetrol 6,8-dibutanoate; (f) [1S-(1α,6β,7α,8β,8aβ)]-octahydro-1,6,7,8-indolizinetetrol 6-butanoate; (g) [1S-(1α,6β,7α,8β,8aβ)]-octahydro-1,6,7,8-indolizinetetrol 6-(2-furancarbonxylate); (h) [1S-(1α,6β,7α,8β,8aβ)]-octahydro-1,6,7,8-indolizinetetrol 7-(2,4-dichlorobenzoate); (i) [1S-(1α,6β,7α,8β,8aβ)]-octahydro-1,6,7,8-indolizinetetrol 6-(3-hexenoate); (j) [1S-(1α,6β,7α,8β,8aβ)]-octahydro-1,6,7,8-indolizinetetrol 6-octanoate; (k) [1S-(1α,6β,7α,8β,8aβ)]-octahydro-1,6,7,8-indolizinetetrol 6-pentanoate; (l) an O-pivaloyl ester; (m) a 2-ethyl-butyryl ester; (n) a 3,3-dimethylbutyryl ester; (o) a cyclopropanoyl ester; (p) a 4-methoxybenzoate ester; (q) a 2-aminobenzoate ester; (r) castanospermine; or (s) a mixture of at least two of (a)-(r). In a preferred embodiment, the glucosidase inhibitor is castanospermine or [1S-(1α,6β,7α,8β,8aβ)]-octahydro-1,6,7,8-indolizinetetrol 6-butanoate (also referred to as celgosivir).

A structurally pure compound refers to a compound composition in which a substantial percentage, e.g., on the order of 95% to 100% and can range from about 95%, 96%, 97%, 98%, 99% or greater, of the individual molecules comprising the composition each contain the same number and types of atoms attached to each other in the same order and with the same bonds. As used herein, the term “structurally pure” is not intended to distinguish different geometric isomers or different optical isomers from one another. For example, a mixture of cis- and trans-but-2,3-ene is considered structurally pure, as is a racemic mixture. When compositions are intended to include a substantial percentage of a single geometric isomer or optical isomer, the terms “geometrically pure” and “optically or enantiomerically pure,” respectively, are used.

The term “structurally pure” is also not intended to discriminate between different tautomeric forms or ionization states of a molecule, or other forms of a molecule that result from equilibrium phenomena or other reversible interconversions. Thus, a composition of, for example, an organic acid is structurally pure even though some of the carboxyl groups may be in a protonated state (COOH) and others may be in a deprotonated state (COO⁻). Likewise, a composition comprising a mixture of keto and enol tautomers, unless specifically noted otherwise, is considered structurally pure.

Combination Therapies and Methods of Use

As described herein, a glucosidase inhibitor (e.g., castanospermine and derivatives thereof) in combination with an agent that alters immune function and an agent that alters viral replication or infectivity, act synergistically to inhibit viral infection or viral replication. In certain embodiments, the combinations described herein are capable of inhibiting replication of a virus of the Flaviviridae family, preferably HCV, at clinically relevant concentrations according to statistically measurable criteria. Use of a glucosidase inhibitor, such as castanospermine or derivatives thereof (e.g., celgosivir) in combination with at least two other therapeutic agents as a treatment encompasses a therapeutic or prophylactic application of the instant disclosure; that is, administration of the combinations to a subject known to be, about to be (at risk), or believed to be infected with a virus of the Flaviviridae family, such as HCV. Also contemplated herein, is a combination of a glucosidase inhibitor (e.g., castanospermine or derivatives thereof) with an agent that alters immune function in a host (e.g., interferon or pegylated interferon, such as interferon-a), an agent that alters viral replication (e.g., ribavirin or 2′-C-methyl cytidine or valopicitabine), or combined with both an agent that alters immune function and an agent that alters viral replication, wherein any embodied combination increases, in a statistically significant and synergistic manner, the effectiveness (efficacy) of the agents for treating a Flaviviridae infection, such as an HCV infection. In still other embodiments, any of these compositions may further optionally comprise an additional adjunctive therapeutic agent, such as an anti-diarrheal agent and the like.

Treatment also encompasses prophylaxis or preventative administration of any combination described herein. Effective treatment of a Flaviviridae infection may include a cure of the infection (i.e., eradication of the virus from the host or host tissue); a sustained response in which HCV RNA is not longer detectable in the blood of the subject six months after completing a therapeutic regimen (such a sustained response may be equated with a favorable prognosis and may be equivalent to a cure); slowing or reducing liver scarring (fibrosis); the slowing or reducing production of the virus; reducing, alleviating, or abrogating symptoms in a subject; or preventing symptoms or infection from worsening or progressing. Thus, the compositions described herein may be used for accomplishing one or more of the following goals: (1) elimination of infectivity and potential transmission of a Flaviviridae infection, such as an HCV infection, to another subject; (2) arresting the progression of liver disease and improving clinical prognosis; (3) preventing development of cirrhosis and HCC; (4) improving the clinical benefit when combined with currently used therapeutic molecules or modalities; or (5) improving the host immune response to HCV infection. To date, a therapeutic agent that adequately treats or prevents an HCV infection, such as genotype 1, and any associated disease without severe side-effects has remained elusive.

In some embodiments, the therapy or prophylaxis may be for the treatment or prevention of disease associated with an infection by a virus, such as Flaviviridae, as described herein. For example, the therapy or prophylaxis may be the treatment or prevention of a disease selected from hepatitis C, yellow fever, dengue fever, Japanese encephalitis, Murray Valley encephalitis, Rocio virus infection, West Nile fever, St. Louis encephalitis, tick-borne encephalitis, Louping ill virus infection, Powassan virus infection, Omsk hemorrhagic fever, Kyasanur forest disease, bovine viral diarrhea, classical swine fever, border disease, and hog cholera. A viral infection, such as a flaviviral infection or an HCV infection, refers to any state or condition that involves (i.e., is caused, exacerbated, or characterized by) a Flaviviridae residing in the cells or body of a subject or patient. A patient or subject may be a human, a non-human primate, sheep, cattle, horse, pig, dog, cat, rat, or mouse, or other mammal.

HCV is difficult to propagate efficiently in cell culture, which renders analysis and identification of potential anti-HCV agents difficult. In the absence of a suitable cell culture system capable of supporting replication of human HCV and re-infection of cells in vitro, use of another member of the Flaviviridae family, bovine viral diarrhea virus (BVDV) is an art-accepted surrogate virus for use in cell culture models (Buckwold et al., Antiviral Res. 60:1, 2003; Stuyver et al., Antimicrob. Agents Chemother. 47:244, 2003; Whitby et al., supra). HCV and BVDV share a significant degree of local protein homology, a common replication strategy, and probably the same subcellular location for viral envelopment. Both HCV and BVDV have single-stranded genomes (approximately 9,600 and 12,600 nucleotides, respectively) that encode nine functionally analogous gene products, including the E1 and E2 envelope glycoproteins (see, e.g., Rice, Flaviviridae: The Viruses and Their Replication, in Fields Virology, 3rd Ed. Philadelphia, Lippincott, 931, 1996). Other assays well-known in the art include HCV pseudoparticles (see, e.g., Bartosch et al., J. Exp. Med. 197:633, 2003; Hsu et al., Proc. Nat'l Acad. Sci. USA 100:7271, 2003) and HCV replicons of any type, such as full length replicons, expressing E1 and E2, and also resistant to IFN-α or ribavirin (see, e.g., U.S. Pat. Nos. 5,372,928; 5,698,446; 5,874,565; 6,750,009).

The compounds described herein may be useful research tools for in vitro and cell-based assays to study the biological mechanisms of viral infection, growth, and replication, such as by HCV. By way of background and not wishing to be bound by theory, HCV morphogenesis is complex wherein preassembled viral core particles are believed to attach to cytosolic sides of viral envelope (surface) proteins, which have inserted in the endoplasmic reticulum (ER) membrane. After acquiring envelopes, virions bud to the lumen of the ER and then are transported through the Golgi apparatus to the extracellular fluids. Removal of N-linked glucose residues (trimming is done by cellular enzymes, such as α-glucosidases) from immature viral glycoproteins may play a role in the migration of viral glycoproteins from the ER to the Golgi.

In one embodiment, a method is provided for identifying anti-viral compounds, comprising contacting a host cell infected with a virus with a glucosidase inhibitor (e.g., castanospermine or a derivative thereof) and at least one other test compound or agent under conditions and for a time sufficient to inhibit viral replication, and identifying a candidate agent that inhibits (prevents, slows, abrogates, interferes with) infection, viral replication, and/or viral assembly. In certain embodiments, the methods described herein are used to identify a test compound that acts synergistically when combined with a glucosidase inhibitor, such as castanospermine or a derivative thereof (e.g., celgosivir). In another embodiment, a method is provided for identifying cells suspected of having a viral infection, comprising contacting a host cell suspected of being infected with a virus with a glucosidase inhibitor (e.g., castanospermine or a derivative thereof) and at least one candidate compound or agent under conditions and for a time sufficient to inhibit infection, viral replication, or viral assembly, and identifying cells infected with a virus. In certain embodiments, the viral infection may be caused by or associated with HCV. The assays described herein are useful for determining the therapeutic value of a candidate compound or combination, and to further determine dosage parameters necessary to effectively treat a subject in need thereof.

In particular embodiments, a glucosidase inhibitor (e.g., castanospermine or a derivative thereof) is administered in association or in combination with an adjunctive therapeutic agent (i.e., in an admixture or co-packaged or administered in such a manner that a glucosidase inhibitor such as castanospermine or a derivative thereof, an agent that alters the host immune function, and an agent that alters viral replication are available systemically or at the site of infection such that the anti-viral effects of the combination is additive or synergistic). In one embodiment, castanospermine or a derivative thereof, such as celgosivir, is combined with an agent that alters immune function, such as interferon-α or pegylated interferon-α, and an agent that alters viral replication, for example, a nucleoside analog such as ribavirin or 2′-C-methyl cytidine or valopicitabine.

A representative adjunctive therapeutic agent can be a compound or molecule that has anti-viral activity may, for example, inhibit or prevent infection of a cell (such as by preventing binding or adherence of the virus to a cell); inhibit, reduce, or prevent viral replication or assembly; inhibit, reduce, or prevent release of viral RNA from the viral capsid; or inhibit, reduce, or interfere with the function of a HCV gene product. Another exemplary adjunctive therapeutic agent can be a compound or molecule that alters immune function (increases or decreases in a statistically significant manner or a clinically significant manner) increases or enhances an immune function or immune response against the infectious virus.

In one embodiment, a composition comprising a glucosidase inhibitor, an agent that alters immune function and an agent that alters viral replication act synergistically in the treatment of infection by Flaviviridae, such as HCV, in a subject or patient. Two or more compounds that act synergistically interact such that the combined effect of the compounds is greater than the sum of the individual effects of each compound when administered alone (see, e.g., Berenbaum, Pharmacol. Rev. 41:93, 1989). For example, an interaction between castanospermine or a derivative thereof and another agent or compound may be analyzed by a variety of mechanistic and empirical models (see, e.g., Ouzounov et al., Antivir. Res. 55:425, 2002). A commonly used approach for analyzing interaction between a combination of agents employs the construction of isoboles (iso-effect curves, also referred to as isobolograms), in which the combination of agents (dₐ,d_{b}) is represented by a point on a graph, the axes of which are the dose-axes of the individual agents (see, e.g., Ouzounov et al., supra; see also Tallarida, J. Pharmacol. Exp. Therap. 298:865, 2001).

Another method for analyzing drug-drug interactions (antagonism, additivity, synergism) known in the art includes determination of combination indices (CI) according to the median effect principle to provide estimates of IC₅₀ values of compounds administered alone and in combination (see, e.g., Chou. In Synergism and Antagonism Chemotherapy. Eds. Chou and Rideout. Academic Press, San Diego Calif., pages 61-102, 1991; CalcuSyn™ software). A CI value of less than one represents synergistic activity, equal to one represents additive activity, and greater than one represents antagonism.

Still another exemplary method is the independent effect method (Pritchard and Shipman, Antiviral Research 14:181, 1990; Pritchard and Shipman, Antiviral Therapy 1:9, 1996; MacSynergy™ II software, University of Michigan, Ann Arbor, Mich.). MacSynergy™ II software allows a three-dimensional (3-D) examination of compound interactions by comparing a calculated additive surface to observed data to generate differential plots that reveal regions (in the form of a volume) of statistically greater than expected (synergy) or less than expected (antagonism) compound interactions. For example, a composition comprising a glucosidase inhibitor, an agent that alters immune function and an agent that alters viral replication will be considered to have synergistic activity or have a synergistic effect when the volume of synergy produced as calculated by the volume of the synergy peaks is about 15% greater than the additive effect (that is, the effect of each agent alone added together), or about a 2-fold to 10-fold greater than the additive effect, or about a 3-fold to 5-fold or more greater than the additive effect.

In certain embodiments, a glucosidase inhibitor (e.g., castanospermine or a derivative thereof, such as celgosivir) in combination with an agent that alters immune function (e.g., interferon) and an agent that alters viral replication (e.g., a nucleoside analogue such as ribavirin or valopicitabine) or another agent or compound described herein may act synergistically or have a synergistic effect when values are between about 25 and 50 μM² % or μM(IU/mL)% (minor but statistically significant); between about 50 and 100 μM²% or μM(IU/mL)% (moderate synergy that may be indicative of a significant synergistic effect in vivo); or greater than about 100 μM²% or IM(IU/mL) % (strong synergy likely indicative of a significant synergistic effect in vivo). Buckwold et al. reported that ribavirin and interferon-α in combination (which is the current standard of combination care for treating HCV infections) had a synergy volume of 66±25 IU(μg)/mL²% (Antimicrob. Agents Chemother. 47:2293, 2003).

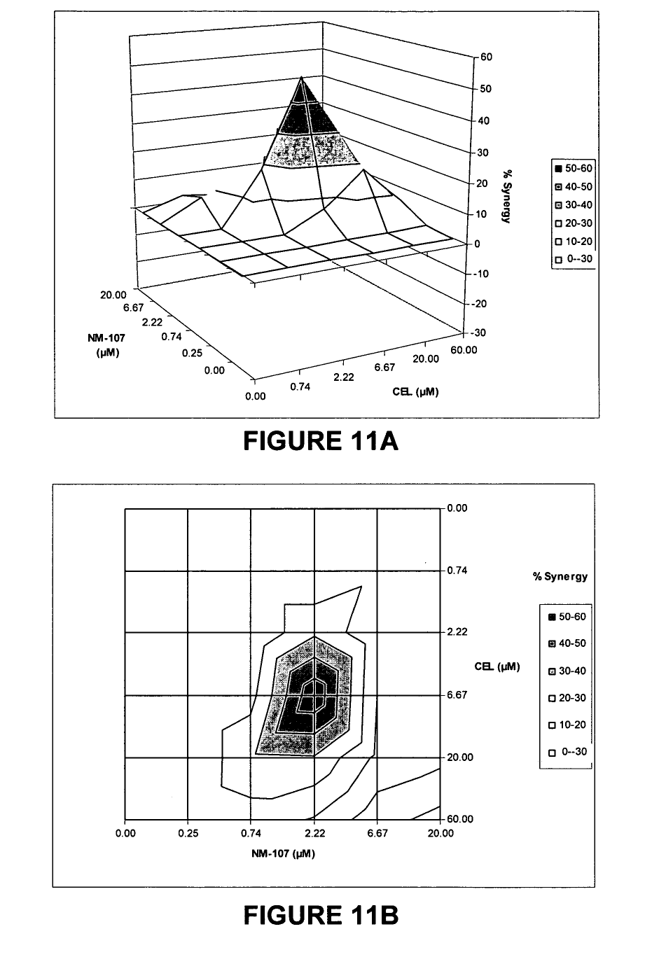

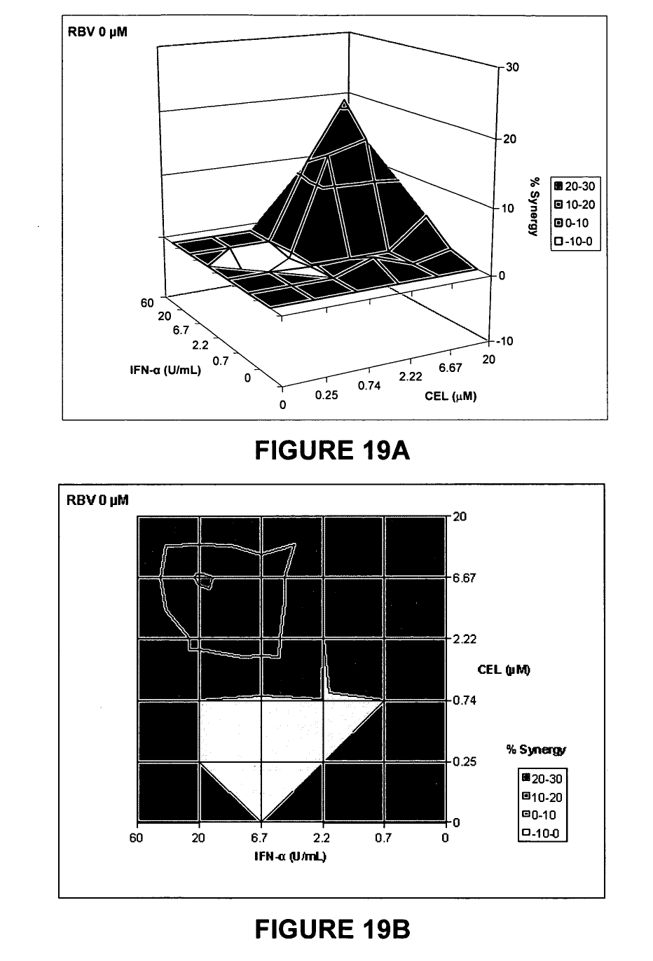

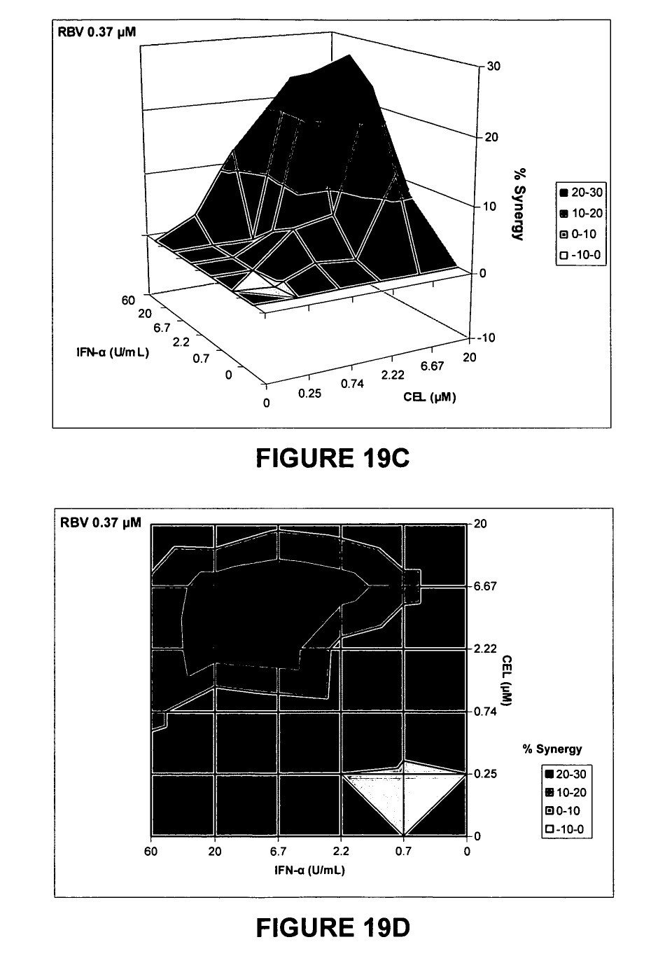

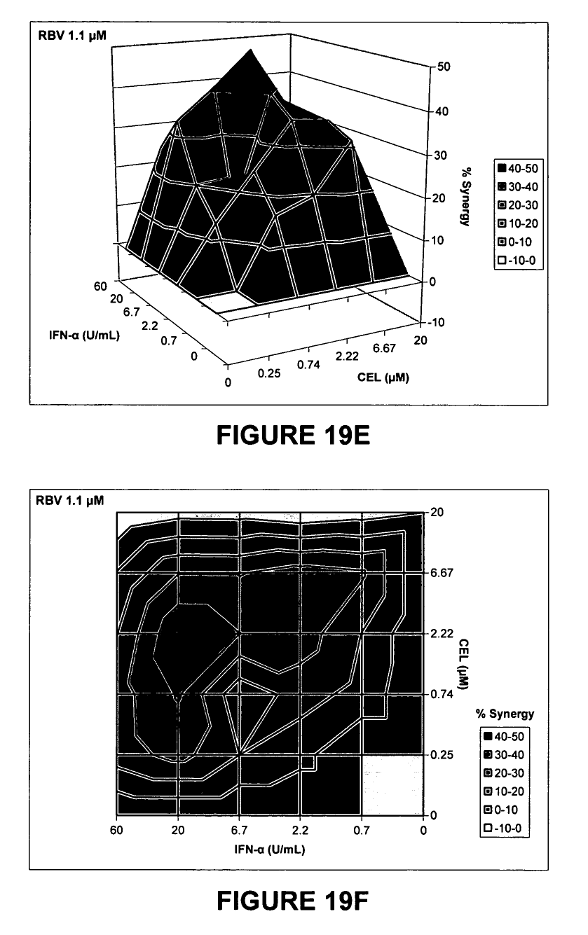

A double combination composition comprising castanospermine or celgosivir, and interferon-α, as described herein, showed a synergy volume ranging from about 96 μM(IU/mL) % to about 168 μM(IU/mL) %, and a triple combination composition comprising castanospermine or celgosivir, ribavirin (0.37 μM to 3.3 μM) and interferon-α, as described herein, showed a synergy volume ranging from about 145 μM(IU/mL) % to about 624 μM(IU/mL) %, and 213 μM(IU/mL) % to about 460 μM(IU/mL) %, respectively (see, e.g., Example 6 and FIG. 19). A double combination comprising celgosivir with 2′-C-methyl cytidine (NM-107, which is the active ingredient of its ester prodrug valopicitabine) also showed a synergistic interaction (see, e.g., Example 3, Table 5 and FIG. 11).

In certain embodiments, a composition of the instant disclosure comprises a glucosidase inhibitor (e.g., castanospermine or a derivative thereof such as celgosivir) in combination with an adjunctive therapeutic agent or compound that inhibits the binding to, or infection of cells, by Flaviviridae (e.g., HCV). Examples of such compounds include antibodies that specifically bind to one or more HCV gene products (e.g., E1 or E2 proteins) or to a cell receptor to which the HCV binds. The antibody may be a monoclonal or polyclonal antibody, or antigen binding fragments thereof, including genetically engineered chimeric, humanized, sFv, or other such immunoglobulins. Other compounds that prevent binding or infection of cells by a virus include glucosaminoglycans (such as heparan sulfate and suramin). In still other embodiments, the combination of a glucosidase inhibitor and a first adjunctive therapeutic agent or compound that inhibits the binding to, or infection of cells, by Flaviviridae is further combined with a second adjunctive therapeutic agent, such as a second glucosidase inhibitor, an agent that alters immune function, an agent that alters Flaviviridae replication, an agent that inhibits the release of Flaviviridae RNA from the viral capsid or inhibits the function of Flaviviridae gene products, an agent that alters symptoms of a Flaviviridae infection, an agent for treating Flaviviridae-associated infections, and the like.

In another embodiment, glucosidase inhibitors of the instant disclosure (e.g., castanospermine or derivatives thereof such as celgosivir) may also be combined with an adjunctive therapeutic agent or compound that inhibits the release of Flaviviridae RNA from the viral capsid or inhibits the function of HCV gene products, including inhibitors of the internal ribosome entry site (IRES), protease inhibitors (e.g., serine protease inhibitors), helicase inhibitors, and inhibitors of the viral polymerase/replicase (see, e.g., Olsen et al., Antimicrob. Agents Chemother. 48:3944, 2004; Stansfield et al., Bioorg. Med. Chem. Lett. 14:5085, 2004). Inhibitors of IRES include, for example, nucleotide sequence specific antisense (see, e.g., McCaffrey et al., Hepatology 38:503, 2003); small yeast RNA (see, e.g., Liang et al., World J. Gastroenterol. 9:1008, 2003); or short interfering RNA molecules (siRNA) that inhibit translation of mRNA; and cyanocobalamin (CNCbl, vitamin B12) (Takyar et al., J. Mol. Biol. 319:1, 2002). NS3 serine protease (helicase) inhibitors include peptides that are derived from NS3 substrates and act to block enzyme activity. Exemplary serine protease inhibitors designated BILN 2061 (see, e.g., Lamarre et al., Nature 426:186, 2003) (Boehringer Ingelheim (Canada) Ltd., Quebec) ,HCV-796 (Wyeth/Viropharma), SCH-503034 (Schering-Plough), ITMN-A (or ITMN-B) (Intermune), and VX-950 (Vertex Pharmaceuticals, Inc. Cambridge, MA) can be combined with glucosidase inhibitors of the instant disclosure, or further combined with additional adjunctive therapeutic agents such as those that alter immune function or that alter Flaviviridae replication. In related embodiments, the combination of a glucosidase inhibitor and a first adjunctive therapeutic agent or compound that inhibits the release of Flaviviridae RNA from the viral capsid or inhibits the function of Flaviviridae gene products is further combined with a second adjunctive therapeutic agent, such as a second glucosidase inhibitor, an agent that alters immune function, an agent that alters Flaviviridae replication, an agent that inhibits the binding to or infection of cells by Flaviviridae, an agent that alters symptoms of a Flaviviridae infection, an agent for treating Flaviviridae-associated infections, and the like.

In another embodiment, glucosidase inhibitors of the instant disclosure (e.g., castanospermine or derivatives thereof, such as celgosivir) may be combined with a compound that perturbs cellular functions involved in or influencing Flaviviridae replication indirectly, such as inhibitors of inosine monophosphate dehydrogenase (e.g., ribavirin, mycophenolic acid, and VX497 (merimepodib, Vertex Pharmaceuticals)), Toll-like receptors (e.g., TLR3, TLR4, TLR7, TLR9) and agonists thereof (such as TLR7 agonists isatoribine or ANA975 (the prodrug of isatoribine) and TLR9 agonist CPG-10101), caspase inhibitors (such as IDN-6556), or inhibitors of HCV p7 (e.g., DGJ and derivatives). Other compounds are those that directly alter Flaviviridae replication, including other inhibitors of glycoprotein processing (such as imino sugars, including deoxygalactonojirimycin (DGJ) and deoxynojirimycin (DNJ), and derivatives thereof (e.g., N-butyl-DNJ, N-nonyl-DNJ, and long alkyl chain imino sugars such as N7-oxanonyl-DNJ, N7-oxanonyl-DGJ)); inhibitors of RNA-dependent RNA polymerase (RdRp inhibitor), such as non-nucleoside analogues (e.g., 2-BAIP) or nucleoside analogues, including 2′-C-methyl cytidine (NM107, Idenix Pharmaceuticals), valopicitabine (NM283, the valine ester prodrug of NM107; Idenix Pharmaceuticals) or the like. NM107 is an active species in cell-based assays and can be delivered to a subject (e.g., humans) as the prodrug NM283. NM107 may be active as is or may be active as a further activated metabolite. Other antiviral compounds can be used as well, such as broad spectrum compounds including amantadine, (Symmetrel®, Endo Pharamceuticals), rimantadine (Flumadine®, Forest Pharmaceuticals, Inc.).

In still other embodiments, the combination of a glucosidase inhibitor and a first adjunctive therapeutic agent or compound that indirectly or directly alters Flaviviridae replication is further combined with a second adjunctive therapeutic agent, such as a second glucosidase inhibitor, an agent that alters immune function, an agent that inhibits the release of Flaviviridae RNA from the viral capsid or inhibits the function of Flaviviridae gene products, an agent that inhibits the binding to or infection of cells by Flaviviridae, an agent that alters symptoms of a Flaviviridae infection, an agent for treating Flaviviridae-associated infections, and the like. In certain embodiments, the combination comprises celgosivir, ribavirin and interferon, or comprises celgosivir, 2′-C-methyl cytidine or valopicitabine and interferon, and optionally DGJ or DNJ.

In another embodiment, glucosidase inhibitors of the instant disclosure (e.g., castanospermine or derivatives thereof such as celgosivir) may be combined with a compound that acts to alter immune function (increase or decrease in a statistically significant, clinically significant, or biologically significant manner), preferably to enhance or stimulate an immune function or an immune response against a Flaviviridae infection. For example, a compound may stimulate a T cell response or enhance a specific immune response (e.g., thymosin-ae such as thymosin-al (e.g., Zadaxin®), and interferons such as α-interferons and β-interferons) or may stimulate or enhance a humoral response. In still other embodiments, the combination of a glucosidase inhibitor and a first adjunctive therapeutic agent or compound that alters immune function is further combined with a second adjunctive therapeutic agent, such as a second glucosidase inhibitor, an agent that alters Flaviviridae replication, an agent that inhibits the release of Flaviviridae RNA from the viral capsid or inhibits the function of Flaviviridae gene products, an agent that inhibits the binding to or infection of cells by Flaviviridae, an agent that alters symptoms of a Flaviviridae infection, an agent for treating Flaviviridae-associated infections, and the like.

Exemplary compounds that alter an immune function include type I interferons, such as interferon-α (see, e.g., Nagata et al., Nature 287:401, 1980), interferon-β (see, e.g., Tanigushi et al., Nature 285:547, 1980), and interferon-ω (Adolf, J. Gen. Virol. 68:1669, 1987); type II interferons, such as interferony (Belardelli, APMIS 103:161, 1995) and interferon-γ-1b (Actimmune®, InterMune); cytokine-like interferons, such as interferon-λ1 (interleukin-29 or IL-29), interferon-λ2 (IL-28A), interferon-λ3 (IL-28B); otherwise unclassified interferons; or the like. Exemplary interferon-α include interferon-α-2a (Roferon®-A; Hoffman-La Roche), interferon-α-2b (Intron A, PBL Biomedical), interferon-α-con-1 (Infergen®, InterMune), interferon-α-n3 (Alferon or Alferon N®, Interferon Sciences), albumin interferon-α (Albuferon-alpha™, Human Genome Sciences, Rockville, Md.) and Veldona (Amarillo Biosciences, Inc.). Exemplary interferon-β include interferon-β-1a (Avonex®, Biogen Idec; or Rebif®, Serono Inc.) and interferon-β-1b (Betaseron®, Berlex).

Interferons alter immune finction and also may alter (inhibit, prevent, abrogate, reduce, or slow) replication of a virus, such as HCV. The production of interferon-α and interferon-β in virally infected cells induces resistance to viral replication, enhances MHC class I expression, increases antigen presentation, and activates natural killer cells (subset of lymphocytes that lack antigen-specific surface receptors) to kill virus-infected cells (see, e.g., Janeway et al., in Immunobiology, 5th ed. New York, London: Garland Publishing, 2001). Thus, these interferons alter immune fimction by affecting both innate and adaptive immunity.

In certain embodiments, castanospermine is administered in combination with the interferon or pegylated interferon, such as pegylated interferon-α2a or pegylated interferon-α2b. Interferon-α has been used in the treatment of a variety of viral infections, either as a monotherapy or as a combination therapy (see, e.g., Liang, New Engl. J. Med. 339:1549, 1998; Hulton et al., J. Acquir. Immune Defic. Syndr. 5:1084, 1992; Johnson et al., J. Infect. Dis. 161:1059, 1990). Interferon-α binds to cell surface receptors and stimulates signal transduction pathways that lead to activation of cellular enzymes (e.g., double-stranded RNA-activated protein kinase and RNase L that inhibit translation initiation and degrade viral RNA, respectively) that repress virus replication (see, e.g, Samuel, Clin. Microbiol. Rev. 14:778, 2001; Kaufman, Proc. Natl. Acad. Sci. USA 96:11693, 1999). HCV E2 glycoprotein and NS5a may block RNA-activated protein kinase activity such that some HCV strains are more resistant to interferon-α; thus, combination therapies of interferon-α and one or more other compounds may be necessary for treatment of persistent viral infection (see, e.g., Ouzounov et al., supra, and references cited therein). In some embodiments, a polyethylene glycol moiety is linked to interferon-α (known as pegylated interferon-a; peginterferon-α-2b (Peg-Intron®; Schering-Plough) and peginterferon-α-2a (Pegasys®; Hoffmann-La Roche)), which have an improved pharmacokinetic profile and also manifest fewer undesirable side effects (see, e.g., Zeuzem et al., New Engl. J. Med. 343:1666, 2000; Heathcote et al., New Engl. J. Med. 343:1673, 2000; Matthews et al., Clin. Ther. 26:991, 2004).

Interferon-α-2a (Roferon®-A; Hoffman-La Roche), Interferon-α-2b (Intron-A; Schering-Plough), and interferon-α-con-1 (Infergeng; InterMune) are approved for use as single agents in the U.S. for treatment of adults with chronic hepatitis C. The recommended dose of interferons-α-2b and -α-2a for the treatment of chronic hepatitis C infection is 3,000,000 units three times a week, administered by subcutaneous or intramuscular injection. Treatment is administered for six months to two years. For interferon-α-con-1, the recommended dose is 9 μg three times a week for first time treatment and 15 μg three times a week for another six months for patients who do not respond or relapse. During the treatment periods with any of these recombinant interferons, the patient must be monitored for side effects, which include flu-like symptoms, depression, rashes, and abnormal blood counts. Treatment with interferon-α alone leads to a sustained response in less than 15% of subjects with genptype 1 infections, so these interferons are rarely used as a monotherapy for the treatment of patients with chronic hepatitis C infection because of this low response rate.

The combination of an interferon-α with ribavirin for treating an HCV infection has been superior to either treatment alone, and the combination is the current standard of care. The effectiveness, doses, and frequency of administration were studied in three large double-blind, placebo-controlled clinical trials (Reichard et al., Lancet 351:83, 1998; Poynard et al., Lancet 352:1426, 1998; McHutchison et al., New Engl. J. Med. 339:1485, 1998; see also Buckwold et al., Antimicrob. Agents Chemother. 47:2293, 2003; Buckhold, J. Antimicrob. Chemother. 53:412, 2004). Adverse effects associated with ribavirin include abnormal fetal development. Ribavirin is also contraindicated in patients who have anemia, heart disease, or kidney disease. Thus, therapeutic doses of ribavirin can be toxic over time.

In one exemplary embodiment, the instant disclosure provides at least a triple combination of a glucosidase inhibitor (e.g., castanospermine or derivatives thereof, celgosivir), an agent that alters immune function (e.g., interferon-α or pegylated interferon-α) and an agent that alters Flaviviridae replication (e.g., ribavirin or 2′-C-methyl cytidine or valopicitabine).

In another embodiment, glucosidase inhibitors, such as castanospermine or derivatives thereof, may be further optionally combined with an adjunctive agent or compound that modulates (preferably decreases or reduces the severity or intensity of, reduces the number of, or abrogates) the symptoms and effects of HCV infection (e.g., antioxidants such as the flavinoids). In another embodiment, the combination of a glucosidase inhibitor and a first adjunctive therapeutic agent or compound that alters symptoms of a Flaviviridae infection is further combined with a second adjunctive therapeutic agent, such as a second glucosidase inhibitor, an agent that alters Flaviviridae replication, an agent that inhibits the release of Flaviviridae RNA from the viral capsid or inhibits the function of Flaviviridae gene products, an agent that inhibits the binding to or infection of cells by Flaviviridae, an agent that alters immune function against Flaviviridae, an agent for treating Flaviviridae-associated infections, and the like.

In certain embodiments, the combination comprises celgosivir, interferon-α2a, and an agent that directly alters Flaviviridae replication. In other embodiments, the combination comprises celgosivir, interferon-α2b, and an agent that directly alters Flaviviridae replication. In still other embodiments, the combination comprises celgosivir, peginterferon-α2a, and an agent that directly alters Flaviviridae replication. In yet other embodiments, the combination comprises celgosivir, peginterferon-α2b, and an agent that directly alters Flaviviridae replication. In further embodiments, the combination comprises celgosivir, interferon-acon-1, and an agent that directly alters Flaviviridae replication. In still further embodiments, the combination comprises celgosivir, interferon-α-n3, and an agent that directly alters Flaviviridae replication. In still other embodiments, the combination comprises celgosivir, interferon-ω, and an agent that directly alters Flaviviridae replication. In other embodiments, the combination comprises celgosivir, interferon-β, and an agent that directly alters Flaviviridae replication. In yet another embodiment, the combination comprises celgosivir, interferon-γ, and an agent that directly alters Flaviviridae replication. In any of these embodiments, the agent that directly alters Flaviviridae replication is an RdRp inhibitor, such as valopicitabine (NM283) or 2′-C-methyl cytidine (NM107). In any of these embodiments, the agent that directly alters Flaviviridae replication is a non-nucleoside analogue, such a 2-BAIP.

In another aspect, the combination comprises castanospermine, interferon-α2a, and an agent that directly alters Flaviviridae replication. In other embodiments, the combination comprises castanospermine, interferon-α2b, and an agent that directly alters Flaviviridae replication. In still other embodiments, the combination comprises castanospermine, peginterferon-α2a, and an agent that directly alters Flaviviridae replication. In yet other embodiments, the combination comprises castanospermine, peginterferon-α2b, and an agent that directly alters Flaviviridae replication. In further embodiments, the combination comprises castanospermine, interferon-αcon-1, and an agent that directly alters Flaviviridae replication. In still further embodiments, the combination comprises castanospermine, interferon-α-n3, and an agent that directly alters Flaviviridae replication. In still other embodiments, the combination comprises castanospermine, interferon-ω, and an agent that directly alters Flaviviridae replication. In other embodiments, the combination comprises castanospermine, interferon-β, and an agent that directly alters Flaviviridae replication. In yet another embodiment, the combination comprises castanospermine, interferon-y, and an agent that directly alters Flaviviridae replication. In any of these embodiments, the agent that directly alters Flaviviridae replication is an RdRp inhibitor, such as valopicitabine (NM283) or 2′-C-methyl cytidine (NM107). In any of these embodiments, the agent that directly alters Flaviviridae replication is a non-nucleoside analogue, such a 2-BAIP.

In certain embodiments, the combination comprises celgosivir, interferon-α2a, and an agent that indirectly alters Flaviviridae replication. In other embodiments, the combination comprises celgosivir, interferon-α2b, and an agent that indirectly alters Flaviviridae replication. In still other embodiments, the combination comprises celgosivir, peginterferon-α2a, and an agent that indirectly alters Flaviviridae replication. In yet other embodiments, the combination comprises celgosivir, peginterferon-α2b, and an agent that indirectly alters Flaviviridae replication. In further embodiments, the combination comprises celgosivir, interferon-acon-1, and an agent that indirectly alters Flaviviridae replication. In still further embodiments, the combination comprises celgosivir, interferon-α-n3, and an agent that indirectly alters Flaviviridae replication. In still other embodiments, the combination comprises celgosivir, interferon-ω, and an agent that indirectly alters Flaviviridae replication. In other embodiments, the combination comprises celgosivir, interferon-β, and an agent that indirectly alters Flaviviridae replication. In yet another embodiment, the combination comprises celgosivir, interferon-γ, and an agent that indirectly alters Flaviviridae replication. In any of these embodiments, the agent that indirectly alters Flaviviridae replication is ribavirin or viramidine.

In certain embodiments, the combination comprises castanospermine, interferon-α2a, and an agent that indirectly alters Flaviviridae replication. In other embodiments, the combination comprises castanospermine, interferon-α2b, and an agent that indirectly alters Flaviviridae replication. In still other embodiments, the combination comprises castanospermine, peginterferon-α2a, and an agent that indirectly alters Flaviviridae replication. In yet other embodiments, the combination comprises castanospermine, peginterferon-α2b, and an agent that indirectly alters Flaviviridae replication. In further embodiments, the combination comprises castanospermine, interferon-αcon-1, and an agent that indirectly alters Flaviviridae replication. In still further embodiments, the combination comprises castanospermine, interferon-α-n3, and an agent that indirectly alters Flaviviridae replication. In still other embodiments, the combination comprises castanospermine, interferon-co, and an agent that indirectly alters Flaviviridae replication. In other embodiments, the combination comprises castanospermine, interferon-β, and an agent that indirectly alters Flaviviridae replication. In yet another embodiment, the combination comprises castanospermine, interferon-γ, and an agent that indirectly alters Flaviviridae replication. In any of these embodiments, the agent that indirectly alters Flaviviridae replication is ribavirin or viramidine.

An adjunctive therapeutic agent may comprise an antiviral compound that is used for treatment of an infectious agent frequently identified as co-infecting a subject who is infected with a Flaviviridae (e.g., HCV), such as an antiviral compound or drug against HBV or HIV. An exemplary co-infection is by HBV, a human retrovirus such as HIV1 and 2, or human T-cell lymphotrophic virus (HTLV) type 1 or type 2, or the like. Exemplary antiviral compounds include nucleotide reverse transcriptase (RT) inhibitors (e.g., lamivudine (3TC), zidovudine, stavudine, didanosine, adefovir dipivoxil, and abacavir); non-nucleoside RT inhibitors (e.g., nevirapine, efavirenz); and protease inhibitors (e.g., saquinavir, indinavir, and ritonavir). In a related embodiment, the combination of a glucosidase inhibitor and a first adjunctive therapeutic agent or compound for treating Flaviviridae-associated infections is further combined with a second adjunctive therapeutic agent, such as a second glucosidase inhibitor, an agent that alters Flaviviridae replication, an agent that inhibits the release of Flaviviridae RNA from the viral capsid or inhibits the function of Flaviviridae gene products, an agent that inhibits the binding to or infection of cells by Flaviviridae, an agent that alters immune function against Flaviviridae, an agent that alters symptoms of a Flaviviridae infection, and the like.

An adjunctive therapeutic may optionally comprise an anti-diarrheal agent, such as an anti-secretory agent, an anti-motility agent, including anticholinergic agents (e.g., agents that increase intestinal transit time or, in other words, decrease peristalsis), an adsorbent agent, a filler agent, or any combination thereof. In certain embodiments, an anti-diarrheal agent may be anti-secretory, such as bismuth subsalicylate. In a further embodiment, an anti-diarrheal agent may be an anti-motility agent, such as loperamide hydrochloride, diphenoxylate hydrochoride, difenoxin hydrochloride, codeine phosphate, or paregoric (camphorated opium tincture). In still further embodiments, an anti-diarrheal agent may be an adsorbent such as attapulgite, kaolin, or pectin. In other embodiments, an anti-diarrheal agent may be an anticholinergic such as belladonna tincture, atropine sulfate, or propantheline. In another embodiment, an anti-diarrheal agent may be a filler or bulk such as calcium polycarbophil. Any one or more of these anti-diarrheal agents may be optionally combined with castanospernine or a derivative thereof, or combined with other adjunctive therapies (such as interferon or ribavirin or valopicitabine) and castanospermine or a derivative thereof. For example, an anti-motility agent (such as diphenoxylate or diphenoxin) and an anticholinergic agent (such as atropine sulfate) can be used in combination with a glucosidase inhibitor (e.g., castanospermine or a derivative thereof, such as celgosivir), or with a combination of a glucosidase inhibitor (such as castanospermine or derivative thereof), an agent that alters immune function (such as interferon or pegylated interferon) and an agent that alters replication of Flaviviridae (such as ribavirin or 2′-C-methyl cytidine or valopicitabine), or any combination thereof.a In certain embodiments, the combination comprises celgosivir, ribavirin and interferon. In other embodiments, the combination comprises celgosivir, amantadine and ribavirin. In certain embodiments, the combination comprises castanospermine, amantadine and 2-BAIP.

In certain embodiments, the combination comprises castanospermine, amantadine and ribavirin. In certain other embodiments, the combination comprises celgosivir, amantadine and viramidine. In further embodiments, the combination comprises castanospermine, amantadine and viramidine. In still other embodiments, the combination comprises celgosivir, amantadine and NM-107. In more embodiments, the combination comprises castanospermine, amantadine and NM-107. In further embodiments, the combination comprises celgosivir, amantadine and NM-283. In still other embodiments, the combination comprises castanospermine, amantadine and NM-283. In additional embodiments, the combination comprises celgosivir, amantadine and 2-BAIP.

In certain embodiments, the combination comprises celgosivir, amantadine and IFN-α2a. In certain embodiments, the combination comprises castanospermine, amantadine and IFN-α2a. In certain embodiments, the combination comprises celgosivir, amantadine and IFN-α2b. In certain embodiments, the combination comprises castanospermine, amantadine and IFN-α2b. In certain embodiments, the combination comprises celgosivir, amantadine and IFN-alfacon-1. In certain embodiments, the combination comprises castanospermine, amantadine and IFN-alfacon-1. In certain embodiments, the combination comprises celgosivir, amantadine and IFN-α-n3. In certain embodiments, the combination comprises castanospermine, amantadine and IFN-α-n3. In certain embodiments, the combination comprises celgosivir, amantadine and IFN-β. In certain embodiments, the combination comprises castanospermine, amantadine and IFN-β. In certain embodiments, the combination comprises celgosivir, amantadine and peg-IFN-α2a. In certain embodiments, the combination comprises castanospermine, amantadine and peg-IFN-α2a. In certain embodiments, the combination comprises celgosivir, amantadine and peg-IFN-α2b. In certain embodiments, the combination comprises castanospermine, amantadine and peg-IFN-α2b. In certain embodiments, the combination comprises celgosivir, amantadine and IFN-omega. In certain embodiments, the combination comprises castanospermine, amantadine and IFN-omega. In certain embodiments, the combination comprises celgosivir, amantadine and IFN-gamma. In certain embodiments, the combination comprises castanospermine, amantadine and IFN-gamma. In certain embodiments, the combination comprises celgosivir, amantadine and IFN-gamma-1b. In certain embodiments, the combination comprises castanospermine, amantadine and IFN-gamma-1b. In certain embodiments, the combination comprises celgosivir, amantadine and IFN-lambda. In certain embodiments, the combination comprises castanospermine, amantadine and IFN-lambda. In certain embodiments, the combination comprises celgosivir, amantadine and NB-DNJ. In certain embodiments, the combination comprises castanospermine, amantadine and NB-DNJ.

In certain embodiments, the combination comprises celgosivir, ribavirin and viramidine. In other embodiments, the combination comprises castanospermine, ribavirin and viramidine. In firther embodiments, the combination comprises celgosivir, ribavirin and NM-107. In certain embodiments, the combination comprises castanospermine, ribavirin and NM-107. In certain embodiments, the combination comprises celgosivir, ribavirin and NM-283. In certain embodiments, the combination comprises castanospermine, ribavirin and NM-283. In certain embodiments, the combination comprises celgosivir, ribavirin and 2-BAIP. In still further embodiments, the combination comprises castanospermine, ribavirin and 2-BAIP. In certain embodiments, the combination comprises celgosivir, ribavirin and IFN-α2a. In certain embodiments, the combination comprises castanospermine, ribavirin and IFN-α2a. In certain embodiments, the combination comprises celgosivir, ribavirin and IFN-α2b. In certain embodiments, the combination comprises castanospermine, ribavirin and IFN-α2b. In certain embodiments, the combination comprises celgosivir, ribavirin and IFN-alfacon-1. In certain embodiments, the combination comprises castanospermine, ribavirin and IFN-alfacon-1. In certain embodiments, the combination comprises celgosivir, ribavirin and IFN-α-n3. In certain embodiments, the combination comprises castanospermine, ribavirin and IFN-α-n3. In certain embodiments, the combination comprises celgosivir, ribavirin and IFN-β. In certain embodiments, the combination comprises castanospermine, ribavirin and IFN-β. In certain embodiments, the combination comprises celgosivir, ribavirin and peg-IFN-α2a. In certain embodiments, the combination comprises castanospermine, ribavirin and peg-IFN-α2a. In certain embodiments, the combination comprises celgosivir, ribavirin and peg-IFN-α2b. In certain embodiments, the combination comprises castanospermine, ribavirin and peg-IFN-α2b. In certain embodiments, the combination comprises celgosivir, ribavirin and IFN-omega. In certain embodiments, the combination comprises castanospermine, ribavirin and IFN-omega. In certain embodiments, the combination comprises celgosivir, ribavirin and IFN-gamma. In certain embodiments, the combination comprises castanospermine, ribavirin and IFN-gamma. In certain embodiments, the combination comprises celgosivir, ribavirin and IFN-gamma-1b. In certain embodiments, the combination comprises castanospermine, ribavirin and IFN-gamma-1b. In certain embodiments, the combination comprises celgosivir, ribavirin and IFN-lambda. In certain embodiments, the combination comprises castanospermine, ribavirin and IFN-lambda. In certain embodiments, the combination comprises celgosivir, ribavirin and NB-DNJ. In certain embodiments, the combination comprises castanospermine, ribavirin and NB-DNJ.

In certain embodiments, the combination comprises celgosivir, viramidine and NM-107. In certain embodiments, the combination comprises castanospermine, viramidine and NM-107. In certain embodiments, the combination comprises celgosivir, viramidine and NM-283. In certain embodiments, the combination comprises castanospermine, viramidine and NM-283. In certain embodiments, the combination comprises celgosivir, viramidine and 2-BAIP. In certain embodiments, the combination comprises castanospermine, viramidine and 2-BAIP. In certain embodiments, the combination comprises celgosivir, viramidine and IFN-α2a. In certain embodiments, the combination comprises castanospermine, viramidine and IFN-α2a. In certain embodiments, the combination comprises celgosivir, viramidine and IFN-α2b. In certain embodiments, the combination comprises castanospermine, viramidine and IFN-α2b. In certain embodiments, the combination comprises celgosivir, viramidine and IFN-alfacon-1. In certain embodiments, the combination comprises Castanospermine, viramidine and IFN-alfacon-1. In certain embodiments, the combination comprises celgosivir, viramidine and IFN-α-n3. In certain embodiments, the combination comprises Castanospermine, viramidine and IFN-α-n3. In certain embodiments, the combination comprises celgosivir, viramidine and IFN-β. In certain embodiments, the combination comprises Castanospermine, viramidine and IFN-β. In certain embodiments, the combination comprises celgosivir, viramidine and peg-IFN-α2a. In certain embodiments, the combination comprises Castanospermine, viramidine and peg-IFN-α2a. In certain embodiments, the combination comprises celgosivir, viramidine and peg-IFN-α2b. In certain embodiments, the combination comprises castanospermine, viramidine and peg-IFN-α2b. In certain embodiments, the combination comprises celgosivir, viramidine and IFN-omega. In certain embodiments, the combination comprises castanospermine, viramidine and IFN-omega. In certain embodiments, the combination comprises celgosivir, viramidine and IFN-gamma. In certain embodiments, the combination comprises castanospermine, viramidine and IFN-gamma. In certain embodiments, the combination comprises celgosivir, viramidine and IFN-gamma-1b. In certain embodiments, the combination comprises castanospermine, viramidine and IFN-gamma-1b. In certain embodiments, the combination comprises celgosivir, viramidine and IFN-lambda. In certain embodiments, the combination comprises castanospermine, viramidine and IFN-lambda. In certain embodiments, the combination comprises celgosivir, viramidine and NB-DNJ. In certain embodiments, the combination comprises castanospermine, viramidine and NB-DNJ.

In certain embodiments, the combination comprises celgosivir, NM-107 and NM-283. In certain embodiments, the combination comprises castanospermine, NM-107 and NM-283. In certain embodiments, the combination comprises celgosivir, NM-107 and 2-BAIP. In certain embodiments, the combination comprises castanospermine, NM-107 and 2-BAIP. In certain embodiments, the combination comprises celgosivir, NM-107 and IFN-α2a. In certain embodiments, the combination comprises castanospermine, NM-107 and IFN-α2a. In certain embodiments, the combination comprises celgosivir, NM-107 and IFN-α2b. In certain embodiments, the combination comprises castanospermine, NM-107 and IFN-α2b. In certain embodiments, the combination comprises celgosivir, NM-107 and IFN-alfacon-1. In certain embodiments, the combination comprises castanospermine, NM-107 and IFN-alfacon-1. In certain embodiments, the combination comprises celgosivir, NM-107 and IFN-α-n3. In certain embodiments, the combination comprises castanospermine, NM-107 and IFN-α-n3. In certain embodiments, the combination comprises celgosivir, NM-107 and IFN-β. In certain embodiments, the combination comprises castanospermine, NM-107 and IFN-β. In certain embodiments, the combination comprises celgosivir, NM-107 and peg-IFN-α2a. In certain embodiments, the combination comprises castanospermine, NM-107 and peg-IFN-α2a. In certain embodiments, the combination comprises celgosivir, NM-107 and peg-IFN-α2b. In certain embodiments, the combination comprises Castanospermine, NM-107 and peg-IFN-α2b. In certain embodiments, the combination comprises celgosivir, NM-107 and IFN-omega. In certain embodiments, the combination comprises Castanospermine, NM-107 and IFN-omega. In certain embodiments, the combination comprises celgosivir, NM-107 and IFN-gamma. In certain embodiments, the combination comprises Castanospermine, NM-107 and IFN-gamma. In certain embodiments, the combination comprises celgosivir, NM-107 and IFN-gamma-1b. In certain embodiments, the combination comprises Castanospermine, NM-107 and IFN-gamma-1b. In certain embodiments, the combination comprises celgosivir, NM-107 and IFN-lambda. In certain embodiments, the combination comprises Castanospermine, NM-107 and IFN-lambda. In certain embodiments, the combination comprises celgosivir, NM-107 and NB-DNJ. In certain embodiments, the combination comprises Castanospermine, NM-107 and NB-DNJ.

In certain embodiments, the combination comprises celgosivir, NM-283 and 2-BAIP. In certain embodiments, the combination comprises Castanospermine, NM-283 and 2-BAIP. In certain embodiments, the combination comprises celgosivir, NM-283 and IFN-α2a. In certain embodiments, the combination comprises Castanospermine, NM-283 and IFN-α2a. In certain embodiments, the combination comprises celgosivir, NM-283 and IFN-α2b. In certain embodiments, the combination comprises Castanospermine, NM-283 and IFN-α2b. In certain embodiments, the combination comprises celgosivir, NM-283 and IFN-alfacon-1. In certain embodiments, the combination comprises Castanospermine, NM-283 and IFN-alfacon-1. In certain embodiments, the combination comprises celgosivir, NM-283 and IFN-α-n3. In certain embodiments, the combination comprises Castanospermine, NM-283 and IFN-α-n3. In certain embodiments, the combination comprises celgosivir, NM-283 and IFN-β. In certain embodiments, the combination comprises Castanospermine, NM-283 and IFN-β. In certain embodiments, the combination comprises celgosivir, NM-283 and peg-IFN-α2a. In certain embodiments, the combination comprises Castanospermine, NM-283 and peg-IFN-α2a. In certain embodiments, the combination comprises celgosivir, NM-283 and peg-IFN-α2b. In certain embodiments, the combination comprises Castanospermine, NM-283 and peg-IFN-α2b. In certain embodiments, the combination comprises celgosivir, NM-283 and IFN-omega. In certain embodiments, the combination comprises Castanospermine, NM-283 and IFN-omega. In certain embodiments, the combination comprises celgosivir, NM-283 and IFN-gamma. In certain embodiments, the combination comprises Castanospermine, NM-283 and IFN-gamma. In certain embodiments, the combination comprises celgosivir, NM-283 and IFN-gamma-1b. In certain embodiments, the combination comprises Castanospermine, NM-283 and IFN-gamma-1b. In certain embodiments, the combination comprises celgosivir, NM-283 and IFN-lambda. In certain embodiments, the combination comprises Castanospermine, NM-283 and IFN-lambda. In certain embodiments, the combination comprises celgosivir, NM-283 and NB-DNJ. In certain embodiments, the combination comprises Castanospermine, NM-283 and NB-DNJ.

In certain embodiments, the combination comprises celgosivir, 2-BAIP and IFN-α2a. In certain embodiments, the combination comprises Castanospermine, 2-BAIP and IFN-α2a. In certain embodiments, the combination comprises celgosivir, 2-BAIP and IFN-α2b. In certain embodiments, the combination comprises Castanospermine, 2-BAIP and IFN-α2b. In certain embodiments, the combination comprises celgosivir, 2-BAIP and IFN-alfacon-1. In certain embodiments, the combination comprises Castanospermine, 2-BAIP and IFN-alfacon-1. In certain embodiments, the combination comprises celgosivir, 2-BAIP and IFN-α-n3. In certain embodiments, the combination comprises Castanospermine, 2-BAIP and IFN-α-n3. In certain embodiments, the combination comprises celgosivir, 2-BAIP and IFN-β. In certain embodiments, the combination comprises Castanospermine, 2-BAIP and IFN-β. In certain embodiments, the combination comprises celgosivir, 2-BAIP and peg-IFN-α2a. In certain embodiments, the combination comprises Castanospermine, 2-BAIP and peg-IFN-α2a. In certain embodiments, the combination comprises celgosivir, 2-BAIP and peg-IFN-α2b. In certain embodiments, the combination comprises Castanospermine, 2-BAIP and peg-IFN-α2b. In certain embodiments, the combination comprises celgosivir, 2-BAIP and IFN-omega. In certain embodiments, the combination comprises Castanospermine, 2-BAIP and IFN-omega. In certain embodiments, the combination comprises celgosivir, 2-BAIP and IFN-gamma. In certain embodiments, the combination comprises Castanospermine, 2-BAIP and IFN-gamma. In certain embodiments, the combination comprises celgosivir, 2-BAIP and IFN-gamma-1b. In certain embodiments, the combination comprises Castanospermine, 2-BAIP and IFN-gamma-1b. In certain embodiments, the combination comprises celgosivir, 2-BAIP and IFN-lambda. In certain embodiments, the combination comprises Castanospermine, 2-BAIP and IFN-lambda. In certain embodiments, the combination comprises celgosivir, 2-BAIP and NB-DNJ. In certain embodiments, the combination comprises Castanospermine, 2-BAIP and NB-DNJ.

In certain embodiments, the combination comprises celgosivir, IFN-α2a and IFN-α2b. In certain embodiments, the combination comprises Castanospermine, IFN-α2a and IFN-α2b. In certain embodiments, the combination comprises celgosivir, IFN-α2a and IFN-alfacon-1. In certain embodiments, the combination comprises Castanospermine, IFN-α2a and IFN-alfacon-1. In certain embodiments, the combination comprises celgosivir, IFN-α2a and IFN-α-n3. In certain embodiments, the combination comprises Castanospermine, IFN-α2a and IFN-α-n3. In certain embodiments, the combination comprises celgosivir, IFN-α2a and IFN-β. In certain embodiments, the combination comprises Castanospermine, IFN-α2a and IFN-β. In certain embodiments, the combination comprises celgosivir, IFN-α2a and peg-IFN-α2a. In certain embodiments, the combination comprises Castanospermine, IFN-α2a and peg-IFN-α2a. In certain embodiments, the combination comprises celgosivir, IFN-α2a and peg-IFN-α2b. In certain embodiments, the combination comprises Castanospermine, IFN-α2a and peg-IFN-α2b. In certain embodiments, the combination comprises celgosivir, IFN-α2a and IFN-omega. In certain embodiments, the combination comprises Castanospermine, IFN-α2a and IFN-omega. In certain embodiments, the combination comprises celgosivir, IFN-α2a and IFN-gamma. In certain embodiments, the combination comprises Castanospermine, IFN-α2a and IFN-gamma. In certain embodiments, the combination comprises celgosivir, IFN-α2a and IFN-gamma-1b. In certain embodiments, the combination comprises Castanospermine, IFN-α2a and IFN-gamma-1b. In certain embodiments, the combination comprises celgosivir, IFN-α2a and IFN-lambda. In certain embodiments, the combination comprises Castanospermine, IFN-α2a and IFN-lambda. In certain embodiments, the combination comprises celgosivir, IFN-α2a and NB-DNJ. In certain embodiments, the combination comprises Castanospermine, IFN-α2a and NB-DNJ.

In certain embodiments, the combination comprises celgosivir, IFN-α2b and IFN-alfacon-1. In certain embodiments, the combination comprises Castanospermine, IFN-α2b and IFN-alfacon-1. In certain embodiments, the combination comprises celgosivir, IFN-α2b and IFN-α-n3. In certain embodiments, the combination comprises Castanospermine, IFN-α2b and IFN-α-n3. In certain embodiments, the combination comprises celgosivir, IFN-α2b and IFN-β. In certain embodiments, the combination comprises Castanospermine, IFN-α2b and IFN-β. In certain embodiments, the combination comprises celgosivir, IFN-α2b and peg-IFN-α2a. In certain embodiments, the combination comprises Castanospermine, IFN-α2b and peg-IFN-α2a. In certain embodiments, the combination comprises celgosivir, IFN-α2b and peg-IFN-α2b. In certain embodiments, the combination comprises Castanospermine, IFN-α2b and peg-IFN-α2b. In certain embodiments, the combination comprises celgosivir, IFN-α2b and IFN-omega. In certain embodiments, the combination comprises Castanospermine, IFN-α2b and IFN-omega. In certain embodiments, the combination comprises celgosivir, IFN-α2b and IFN-gamma. In certain embodiments, the combination comprises Castanospermine, IFN-α2b and IFN-gamma. In certain embodiments, the combination comprises celgosivir, IFN-α2b and IFN-gamma-1b. In certain embodiments, the combination comprises Castanospermine, IFN-α2b and IFN-gamma-1b. In certain embodiments, the combination comprises celgosivir, IFN-α2b and IFN-lambda. In certain embodiments, the combination comprises Castanospermine, IFN-α2b and IFN-lambda. In certain embodiments, the combination comprises celgosivir, IFN-α2b and NB-DNJ. In certain embodiments, the combination comprises Castanospermine, IFN-α2b and NB-DNJ.

In certain embodiments, the combination comprises celgosivir, IFN-alfacon-1 and IFN-α-n3. In certain embodiments, the combination comprises Castanospermine, IFN-alfacon-1 and IFN-α-n3. In certain embodiments, the combination comprises celgosivir, IFN-alfacon-1 and IFN-β. In certain embodiments, the combination comprises Castanospermine, IFN-alfacon-1 and IFN-β. In certain embodiments, the combination comprises celgosivir, IFN-alfacon-1 and peg-IFN-α2a. In certain embodiments, the combination comprises Castanospermine, IFN-alfacon-1 and peg-IFN-α2a. In certain embodiments, the combination comprises celgosivir, IFN-alfacon-1 and peg-IFN-α2b. In certain embodiments, the combination comprises Castanospermine, IFN-alfacon-1 and peg-IFN-α2b. In certain embodiments, the combination comprises celgosivir, IFN-alfacon-1 and IFN-omega. In certain embodiments, the combination comprises Castanospermine, IFN-alfacon-1 and IFN-omega. In certain embodiments, the combination comprises celgosivir, IFN-alfacon-1 and IFN-gamma. In certain embodiments, the combination comprises Castanospermine, IFN-alfacon-1 and IFN-gamma. In certain embodiments, the combination comprises celgosivir, IFN-alfacon-1 and IFN-gamma-1b. In certain embodiments, the combination comprises Castanospermine, IFN-alfacon-1 and IFN-gamma-1b. In certain embodiments, the combination comprises celgosivir, IFN-alfacon-1 and IFN-lambda. In certain embodiments, the combination comprises Castanospermine, IFN-alfacon-1 and IFN-lambda. In certain embodiments, the combination comprises celgosivir, IFN-alfacon-1 and NB-DNJ. In certain embodiments, the combination comprises Castanospermine, IFN-alfacon-1 and NB-DNJ.

In certain embodiments, the combination comprises Celgosivir, IFN-α-n3 and IFN-β. In certain embodiments, the combination comprises Castanospermine, IFN-α-n3 and IFN-β. In certain embodiments, the combination comprises Celgosivir, IFN-α-n3 and peg-IFN-α2a. In certain embodiments, the combination comprises Castanospermine, IFN-α-n3 and peg-IFN-α2a. In certain embodiments, the combination comprises celgosivir, IFN-α-n3 and peg-IFN-α2b. In certain embodiments, the combination comprises Castanospermine, IFN-α-n3 and peg-IFN-α2b. In certain embodiments, the combination comprises celgosivir, IFN-α-n3 and IFN-omega. In certain embodiments, the combination comprises Castanospermine, IFN-α-n3 and IFN-omega. In certain embodiments, the combination comprises celgosivir, IFN-α-n3 and IFN-gamma. In certain embodiments, the combination comprises Castanospermine, IFN-α-n3 and IFN-gamma. In certain embodiments, the combination comprises celgosivir, IFN-α-n3 and IFN-gamma-1b. In certain embodiments, the combination comprises Castanospermine, IFN-α-n3 and IFN-gamma-1b. In certain embodiments, the combination comprises celgosivir, IFN-α-n3 and IFN-lambda. In certain embodiments, the combination comprises Castanospermine, IFN-α-n3 and IFN-lambda. In certain embodiments, the combination comprises celgosivir, IFN-α-n3 and NB-DNJ. In certain embodiments, the combination comprises Castanospermine, IFN-α-n3 and NB-DNJ.

In certain embodiments, the combination comprises celgosivir, IFN-β and peg-IFN-α2a. In certain embodiments, the combination comprises castanospermine, IFN-β and peg-IFN-α2a. In certain embodiments, the combination comprises celgosivir, IFN-β and peg-IFN-α2b. In certain embodiments, the combination comprises Castanospermine, IFN-β and peg-IFN-α2b. In certain embodiments, the combination comprises celgosivir, IFN-β and IFN-omega. In certain embodiments, the combination comprises Castanospermine, IFN-β and IFN-omega. In certain embodiments, the combination comprises celgosivir, IFN-β and IFN-gamma. In certain embodiments, the combination comprises castanospermine, IFN-β and IFN-gamma. In certain embodiments, the combination comprises celgosivir, IFN-β and IFN-gamma-1b. In certain embodiments, the combination comprises Castanospermine, IFN-β and IFN-gamma-1b. In certain embodiments, the combination comprises celgosivir, IFN-β and IFN-lambda. In certain embodiments, the combination comprises Castanospermine, IFN-β and IFN-lambda. In certain embodiments, the combination comprises celgosivir, IFN-β and NB-DNJ. In certain embodiments, the combination comprises Castanospermine, IFN-β and NB-DNJ.

In certain embodiments, the combination comprises celgosivir, peg-IFN-α2a and peg-IFN-α2b. In certain embodiments, the combination comprises Castanospermine, peg-IFN-α2a and peg-IFN-α2b. In certain embodiments, the combination comprises celgosivir, peg-IFN-α2a and IFN-omega. In certain embodiments, the combination comprises Castanospermine, peg-IFN-α2a and IFN-omega. In certain embodiments, the combination comprises celgosivir, peg-IFN-α2a and IFN-gamma. In certain embodiments, the combination comprises Castanospermine, peg-IFN-α2a and IFN-gamma. In certain embodiments, the combination comprises celgosivir, peg-IFN-α2a and IFN-gamma-1b. In certain embodiments, the combination comprises Castanospermine, peg-IFN-α2a and IFN-gamma-1b. In certain embodiments, the combination comprises celgosivir, peg-IFN-α2a and IFN-lambda. In certain embodiments, the combination comprises Castanospermine, peg-IFN-α2a and IFN-lambda In certain embodiments, the combination comprises celgosivir, peg-IFN-α2a and NB-DNJ. In certain embodiments, the combination comprises Castanospermine, peg-IFN-α2a and NB-DNJ.

In certain embodiments, the combination comprises celgosivir, peg-IFN-α2b and IFN-omega. In certain embodiments, the combination comprises Castanospermine, peg-IFN-α2b and IFN-omega. In certain embodiments, the combination comprises celgosivir, peg-IFN-α2b and IFN-gamma. In certain embodiments, the combination comprises Castanospermine, peg-IFN-α2b and IFN-gamma. In certain embodiments, the combination comprises celgosivir, peg-IFN-α2b and IFN-gamma-1b. In certain embodiments, the combination comprises Castanospermine, peg-IFN-α2b and IFN-gamma-1b. In certain embodiments, the combination comprises celgosivir, peg-IFN-α2b and IFN-lambda. In certain embodiments, the combination comprises castanospermine, peg-IFN-α2b and IFN-lambda. In certain embodiments, the combination comprises celgosivir, peg-IFN-α2b and NB-DNJ. In certain embodiments, the combination comprises castanospermine, peg-IFN-α2b and NB-DNJ.

In certain embodiments, the combination comprises celgosivir, IFN-omega and IFN-gamma. In certain embodiments, the combination comprises castanospermine, IFN-omega and IFN-gamma. In certain embodiments, the combination comprises celgosivir, IFN-omega and IFN-gamma-1b. In certain embodiments, the combination comprises castanospermine, IFN-omega and IFN-gamma-1b. In certain embodiments, the combination comprises celgosivir, IFN-omega and IFN-lambda. In certain embodiments, the combination comprises castanospermine, IFN-omega and IFN-lambda. In certain embodiments, the combination comprises celgosivir, IFN-omega and NB-DNJ. In certain embodiments, the combination comprises castanospermine, IFN-omega and NB-DNJ.

In certain embodiments, the combination comprises celgosivir, IFN-gamma and IFN-gamma-1b. In certain embodiments, the combination comprises castanospermine, IFN-gamma and IFN-gamma-1b. In certain embodiments, the combination comprises celgosivir, IFN-gamma and IFN-lambda. In certain embodiments, the combination comprises castanospermine, IFN-gamma and IFN-lambda. In certain embodiments, the combination comprises celgosivir, IFN-gamma and NB-DNJ. In certain embodiments, the combination comprises Castanospermine, IFN-gamma and NB-DNJ.

In certain embodiments, the combination comprises celgosivir, IFN-gamma-1b and IFN-lambda. In certain embodiments, the combination comprises Castanospermine, IFN-gamma-1b and IFN-lambda. In certain embodiments, the combination comprises celgosivir, IFN-gamma-1b and NB-DNJ. In certain embodiments, the combination comprises Castanospermine, IFN-gamma-1b and NB-DNJ.

In certain embodiments, the combination comprises celgosivir, IFN-lambda and NB-DNJ. In certain embodiments, the combination comprises castanospermine, IFN-lambda and NB-DNJ.

In certain embodiments, the combinations of compounds may be administered concurrently, together in the same pharmaceutically acceptable carrier, or separately (but concurrently). In other embodiments, the glucosidase inhibitor and adjunctive therapeutic(s) can be sequentially administered, and sequentially administered in any order or combination.

Any of the specific combinations of compounds disclosed herein may be synergistic and may be used in a method for treating a Flaviviridae infection.

In certain embodiments, the combination comprises celgosivir, interferon-α2a, and an agent that directly alters Flaviviridae replication, wherein celgosivir and the agent that directly alters Flaviviridae replication are administered orally and the interferon-α2a is administered by injection, such as injection subcutaneously. In other embodiments, the combination comprises celgosivir, interferon-α2b, and an agent that directly alters Flaviviridae replication, wherein celgosivir and the agent that directly alters Flaviviridae replication are administered orally and the interferon-α2b is administered by injection, such as injection subcutaneously. In still other embodiments, the combination comprises celgosivir, peginterferon-α2a, and an agent that directly alters Flaviviridae replication, wherein celgosivir and the agent that directly alters Flaviviridae replication are administered orally and the peginterferon-α2a is administered by injection, such as injection subcutaneously. In yet other embodiments, the combination comprises celgosivir, peginterferon-α2b, and an agent that directly alters Flaviviridae replication, wherein celgosivir and the agent that directly alters Flaviviridae replication are administered orally and the peginterferon-α2b is administered by injection, such as injection subcutaneously. In further embodiments, the combination comprises celgosivir, interferon-acon-1, and an agent that directly alters Flaviviridae replication, wherein celgosivir and the agent that directly alters Flaviviridae replication are administered orally and the interferon-acon-l is administered by injection, such as injection subcutaneously. In still further embodiments, the combination comprises celgosivir, interferon-α-n3, and an agent that directly alters Flaviviridae replication, wherein celgosivir and the agent that directly alters Flaviviridae replication are administered orally and the interferon-α-n3 is administered by injection, such as injection subcutaneously. In still other embodiments, the combination comprises celgosivir, interferon-ω, and an agent that directly alters Flaviviridae replication, wherein celgosivir and the agent that directly alters Flaviviridae replication are administered orally and the interferon-ω is administered by injection, such as injection subcutaneously. In other embodiments, the combination comprises celgosivir, interferon-β, and an agent that directly alters Flaviviridae replication, wherein celgosivir and the agent that directly alters Flaviviridae replication are administered orally and the interferon-β is administered by injection, such as injection subcutaneously. In yet another embodiment, the combination comprises celgosivir, interferon-γ, and an agent that directly alters Flaviviridae replication, wherein celgosivir and the agent that directly alters Flaviviridae replication are administered orally and the interferon-γ is administered by injection, such as injection subcutaneously. In any of these embodiments, the agent that directly alters Flaviviridae replication is an RdRp inhibitor, such as valopicitabine (NM283) or 2′-C-methyl cytidine (NM107). In any of these embodiments, the agent that directly alters Flaviviridae replication is a non-nucleoside analogue, such a 2-BAIP. In any of these embodiments, the combinations of compounds may be administered concurrently, sequentially, or sequentially in any order or combination thereof.

In certain embodiments, the combination comprises castanospermine, interferon-α2a, and an agent that directly alters Flaviviridae replication, wherein castanospermine and the agent that directly alters Flaviviridae replication are administered orally and the interferon-α2a is administered by injection, such as injection subcutaneously. In other embodiments, the combination comprises castanospermine, interferon-α2b, and an agent that directly alters Flaviviridae replication, wherein castanospermine and the agent that directly alters Flaviviridae replication are administered orally and the interferon-α2b is administered by injection, such as injection subcutaneously. In still other embodiments, the combination comprises castanospermine, peginterferon-α2a, and an agent that directly alters Flaviviridae replication, wherein castanospermine and the agent that directly alters Flaviviridae replication are administered orally and the peginterferon-α2a is administered by injection, such as injection subcutaneously. In yet other embodiments, the combination comprises castanospermine, peginterferon-α2b, and an agent that directly alters Flaviviridae replication, wherein castanospermine and the agent that directly alters Flaviviridae replication are administered orally and the peginterferon-α2b is administered by injection, such as injection subcutaneously. In further embodiments, the combination comprises castanospermine, interferon-αcon-1, and an agent that directly alters Flaviviridae replication, wherein castanospermine and the agent that directly alters Flaviviridae replication are administered orally and the interferon-αcon-1 is administered by injection, such as injection subcutaneously. In still further embodiments, the combination comprises castanospermine, interferon-α-n3, and an agent that directly alters Flaviviridae replication, wherein castanospermine and the agent that directly alters Flaviviridae replication are administered orally and the interferon-α-n3 is administered by injection, such as injection subcutaneously. In still other embodiments, the combination comprises castanospermine, interferon-ω, and an agent that directly alters Flaviviridae replication, wherein castanospermine and the agent that directly alters Flaviviridae replication are administered orally and the interferon-ω is administered by injection, such as injection subcutaneously. In other embodiments, the combination comprises castanospermine, interferon-β, and an agent that directly alters Flaviviridae replication, wherein castanospermine and the agent that directly alters Flaviviridae replication are administered orally and the interferon-β is administered by injection, such as injection subcutaneously. In yet another embodiment, the combination comprises castanospermine, interferon-γ, and an agent that directly alters Flaviviridae replication, wherein castanospermine and the agent that directly alters Flaviviridae replication are administered orally and the interferon-γ is administered by injection, such as injection subcutaneously. In any of these embodiments, the agent that directly alters Flaviviridae replication is an RdRp inhibitor, such as valopicitabine (NM283) or 2′-C-methyl cytidine (NM107). In any of these embodiments, the agent that directly alters Flaviviridae replication is a non-nucleoside analogue, such a 2-BAIP. In any of these embodiments, the combinations of compounds may be administered concurrently, sequentially, or sequentially in any order or combination thereof.

Methods for determining the effects of castanospermine or a derivative thereof and each of the aforementioned adjunctive therapeutics, that is, for example, altering an immune response, modulating symptoms and effects of a Flaviviridae infection, or altering viral replication (preferably adversely affecting, preventing, decreasing, or inhibiting viral replication), may be carried out by methods described herein and routinely practiced by a skilled artisan.

As described herein, BVDV is an art-accepted surrogate virus for use in cell culture models (Buckwold et al. supra; Stuyver et al., supra; Whitby et al., supra). Assays may therefore be performed using bovine cell lines, such as bovine kidney cells (MDBK) and bovine turbinate (BT) cells, using a cytopathic strain of BVDV such as the NADL strain (available from ATCC, Manassas, Va.) that causes cytolysis of infected cells. Exemplary assays that may be performed to determine whether castanospermine or a derivative thereof alone or in combination with another compound, agent, or molecule may be useful for treating a Flaviviridae infection or inhibiting or preventing a Flaviviridae infection include viral plaque formation assays, cytotoxicity assays (see, e.g., Buckwold et al., Antimicrob. Agents Chemother. 47:2293, 2003; Whitby et al., supra), virus release assays, cell proliferation assays (e.g., nonradioactive MTS/PMS or MTT assays, or radioactive thymidine incorporation assays), and other assays described herein and known and practiced by persons skilled in the art. The data from these assays when castanospermine are analyzed in combination with another compound, such as data obtained from the cytotoxicity assay, may be analyzed as described herein to determine whether the agents interact to provide an additive effect or a synergistic effect.

This disclosure also relates to pharmaceutical compositions that contain a glucosidase inhibitor (e.g., castanospermine or a derivative thereof, such as celgosivir) in combination with one or more compounds used to treat or prevent a viral infection (e.g., HCV). The instant disclosure further relates to methods for treating or preventing viral infections by administering to a subject castanospermine or a derivative thereof in combination with at least two other agents or compounds, wherein each component is administered at a dose sufficient to treat or prevent a viral infection, as described herein. The castanospermine or derivatives thereof and combinations or cocktails of such compounds, are preferably part of a pharmaceutical composition when used in the methods described herein. A castanospermine or a derivative thereof (e.g., celgosivir) may be administered in combination with another compound described herein by administering each compound sequentially to a subject, that is, castanospermine or a derivative thereof may be administered prior to administration of another compound, after administration of another compound; alternatively castanospermine or a derivative thereof (such as celgosivir) may be administered concurrently with another compound. For sequential or concurrent administration of each compound (molecule, agent) of a combination described herein, each compound may be administered by the same or different routes in the same or different formulations, which are described herein and determined, in part, according to the properties of the compounds.

In one embodiment, the invention comprises a pharmaceutical composition comprising a glucosidase inhibitor as described herein (or a pharmaceutical salt thereof) with an adjunctive therapy and a pharmaceutically acceptable carrier, vehicle or excipient, and optional additives (e.g., one or more binders, colorings, desiccants, stabilizers, diluents, preservatives or other adjunctive therapeutics) for use in the methods of treatment described herein. Pharmaceutical compositions comprising interferon-α and ribavirin may be prepared according to methods known and practiced in the art for preparing these compounds for administration to a subject.

As set forth herein, castanospermine or a derivative thereof (e.g., celgosivir) and two or more adjunctive therapeutic compounds or agents may be included in a pharmaceutically acceptable carrier, excipient or diluent for administration to a subject in need thereof in an amount effective to treat or prevent a Flaviviridae infection, such as an HCV infection. In one exemplary embodiment, the instant disclosure provides a glucosidase inhibitor (e.g., castanospermine or derivatives thereof, celgosivir), an agent that alters immune function (e.g., interferon-α or pegylated interferon-α) and an agent that alters Flaviviridae replication (e.g., ribavirin or valopicitabine or 2′-C-methyl cytidine) in a pharmaceutically acceptable carrier, excipient or diluent.

In certain embodiments, a dose of the active compound(s) for the indications described herein may be in a range from about 0.01 mg/kg to about 300 mg/kg per day; preferably about 0.1 mg/kg to about 100 mg/kg per day, more preferably about 0.5 mg/kg to about 25 mg/kg body weight of the recipient per day. In some embodiments, a topical dosage can range from about 0.01-3% wt/wt in a suitable carrier. Interferon-α or ribavirin when administered in combination with castanospermine or a derivative thereof may be administered according to dosing regimens known and practiced in the art (see, e.g., Matthews et al., supra; Foster, Semin. Liver Dis. 24 Suppl 2:97, 2004; Craxi et al., Semin. Liver Dis. 23 Suppl 1:35, 2003).

In addition, the dose of one or more adjunctive therapeutic agents may be adjusted away from the norm when administered with castanospermine or a derivative thereof. For example, due to the reduced cytotoxicity and the synergy seen when interferon and/or ribavirin are combined with castanospermine or celgosivir (i.e., results in a subtherapeutic dose effect) the dosages may be adjusted so that more IFN-α or ribavirin may be safely administered. As used herein, a “subtherapeutic dose effect” means a dose of a therapeutic compound (e.g., glucosidase inhibitor, agent that alters immune function, agent that alters Flaviviridae replication directly or indirectly, or any combination thereof) that is the same or higher than the usual or typical dose of the therapeutic compound administered alone for the treatment of a Flaviviridae infection but shows no increase in adverse side effect or even a decrease in side effects or associated adverse events (i.e., mimics the effects seen at subtherapeutic levels). The castanospermine or a derivative thereof (e.g., celgosivir) may also be adjusted.

Alternatively, lower (subtherapeutic) doses of IFN-α or ribavirin or both in combination with castanospermine or celgosivir may be used with the same effectiveness and less toxicity as higher doses of the IFN-α or ribavirin administered individually or together. As used herein, “subtherapeutic dose” means a dose of a therapeutic compound (e.g., glucosidase inhibitor, agent that alters immune function, agent that alters Flaviviridae replication directly or indirectly, or any combination thereof) that is lower than the usual or typical dose of the therapeutic compound when administered alone for the treatment of a Flaviviridae infection.

The active ingredient(s) are preferably administered to achieve peak plasma concentrations of about 0.001 μM to about 30 μM, and preferably about 0.01 μM to about 10 μM. This may be achieved, for example, by intravenous injection of a composition of a formulation of castanospermine or a derivative thereof, optionally in saline or other aqueous medium. In another embodiment, castanospermine is administered as a bolus. Castanospermine or a derivative thereof (e.g., celgosivir) and other compounds used in the methods of treatment described herein may be administered orally, or intramuscularly, intraperitoneally, intravenously, subcutaneously, transdermally, via an aerosol or by inhalation, rectally, vaginally, or topically (including buccal and sublingual administration).

The concentration of an active compound in a pharmaceutical composition will depend on absorption, distribution, inactivation (e.g., metabolism), and excretion rates of the compound, as well as other factors known to those of skill in the art. The dose will also vary with the severity of the condition to be alleviated. Specific dose regimens (including frequency of dose administration) may be adjusted over time according to the individual subject's need and the professional judgment of the person administering or supervising the administration of the compositions. The dose level and regimen will depend on a variety of factors, including the age, body weight, diet, gender, general health, medical history (including whether the subject is co-infected with another virus, such as HBV or HIV). In certain embodiments, a single dose may be sufficient to obtain a desired clinical outcome. Accordingly, the concentration ranges set forth herein are exemplary only and are not intended to limit the scope or practice of the claimed compositions. For example, the active ingredient may be administered all at once, or may be divided into a number of smaller doses to be administered at varying intervals of time.

The compositions for pharmaceutical use as described herein may be in the form of a kit of parts. The kit may comprise, for example, a glucosidase inhibitor (e.g., castanospermine or a derivative thereof, such as celgosivir), as one component of the composition in unit dosage form, and comprises an agent that alters immune function (e.g., interferon or pegylated interferon) and comprises an agent that alters viral replication (such as ribavirin or valopicitabine or 2′-C-methyl cytidine), each in the respective dosage unit form. The kit may include instructions for use and other relevant information, as well as information required by a regulatory agency.

Oral compositions will generally include an inert diluent or an edible carrier. They may be enclosed in gelatin capsules, compressed into tablets, or made into other oral forms. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as fatty oil. In addition, dosage unit forms can contain various other materials that modify the physical form of the dosage unit, for example, coatings of sugar, shellac, or enteric agents. See generally “Remington's Pharmaceutical Sciences,” Mack Publishing Co., Easton, Pa.

The active compound or pharmaceutically acceptable salt or derivative thereof can be administered as a component of an elixir, suspension, syrup, wafer, chewing gum or the like. Syrup may contain, in addition to the active compounds, sucrose as a sweetening agent and certain preservatives, dyes and colorings, and flavors.

The pharmaceutical composition described herein will preferably include at least one of a pharmaceutically acceptable vehicle, carrier, diluent or excipient, in addition to castanospermine or a derivative thereof, and other components or active ingredients (such as other anti-HCV drug), including agents that alter viral replication or alter an immune function or response, or an agent that is an anti-Hepadnaviridae (e.g., anti-HBV), which are described in detail herein. A composition of the invention may have a variety of active ingredients, such as castanospermine or a derivative thereof, or pharmaceutically acceptable salts thereof, or a cocktail or combination with one or more anti-diarrheal agents, antibiotics, anti-fungals, anti-inflammatory agents, or other anti-viral compounds as described herein (including gastrointestinal anti-motility agents, interferons, cytokines, nucleoside analogs, and the like).

Pharmaceutically acceptable carriers suitable for use with a composition may include, for example, a thickening agent, a buffering agent, a solvent, a humectant, a preservative, a chelating agent, an adjuvant, and the like, and combinations thereof. Pharmaceutically acceptable carriers for therapeutic use are well known in the pharmaceutical art, and as described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; anti-bacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes, or multiple dose vials made of glass or plastic. If administered intravenously, preferred carriers are physiological saline or phosphate buffered saline (PBS) or an adjuvant. Exemplary adjuvants are alum (aluminum hydroxide, REHYDRAGEL®); aluminum phosphate; virosomes, liposomes with and without Lipid A, Detox (Ribi/Corixa); MF59; or other oil and water emulsions type adjuvants, such as nanoemulsions (see, e.g., U.S. Pat. No. 5,716,637) and submicron emulsions (see, e.g., U.S. Pat. No. 5,961,970), and Freund's complete and incomplete. In certain embodiments, a pharmaceutical composition is sterile.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. For example, as is known in the art, some of these materials can be obtained commercially from Alza Corporation (CA) and Gilford Pharmaceuticals (Baltimore, Md.).

Liposomal suspensions may also be pharmaceutically acceptable carriers. These may be prepared according to methods known to those skilled in the art (for example, U.S. Pat. Nos. 4,522,811; 6,320,017; 5,595,756). For example, liposome formulations may be prepared by dissolving appropriate lipid(s) (such as stearoyl phosphatidyl ethanolamine, stearoyl phosphatidyicholine, arachadoyl phosphatidylcholine, and cholesterol) in an inorganic solvent that is then evaporated, leaving behind a thin film of dried lipid on the surface of the container. An aqueous solution of the active compound or its monophosphate, diphosphate, or triphosphate derivatives is then introduced into the container. The container is then swirled by hand to free lipid material from the sides of the container and to disperse lipid aggregates, thereby forming the liposomal suspension. Hydrophilic compounds, such as castanospermine or a derivative thereof like celgosivir, may likely be loaded into the aqueous interior of a liposome.

All U.S. patents, U.S. patent application publications, U.S. patent applications, foreign patents, foreign patent applications, and non-patent publications referred to in this specification, are incorporated herein by reference, in their entirety. The following examples are intended to illustrate, but not limit, the invention.

### EXAMPLES

### Example 1

### In Vitro Inhibition of Viral Release from BVDV-Infected MDBK Cells

Madin-Darby Bovine Kidney Cells (MDBK) (American Type Culture Collection (ATCC), Manassas, Va.; ATCC CCL22) were seeded into 96-well plates at a density of approximately 2×10⁴ cells per well in Dulbecco's Modified Eagles Medium (DMEM/F12; Gibco, Ontario, Canada) containing 2% heat inactivated horse serum (HS, Sigma Aldrich). The cell cultures were incubated at 37° C., 5% CO₂ for about 24 hours to allow attachment of the cells to the tissue culture plates prior to infection and treatment with the test compounds. The cells were infected with sufficient plaque forming units (PFUs) of BVDV strain NADL (ATCC VR-534) diluted in sterile phosphate buffered saline (PBS) containing 1% HS and 1 mM MgCl₂ to achieve a desired multiplicity of infection (MOI) (about 1 virus per cell), incubated at 37° C., 5% CO₂ for about 1 to 2 hours, and then washed with PBS. The infected cells were then suspended in cell growth medium, 2% HS alone or containing one test compound at varying concentrations, and then incubated at 37° C. under 5% CO₂ for 24 hours (i.e., one cycle of BVDV replication). The following test compounds were used: (1) celgosivir; (2) castanospermine (Phytex, Australia); (3) ribavirin (Sigma); and (4) Interferon-α2b (IFN-α2b; PBL Biomedical Laboratories, Piscataway, N.J.). The 96-well plates containing the treated cells were then centrifuged at low speed to sediment any loose cells or debris, the supernatant was harvested and serially diluted to infect a new monolayer of cells in 12-well plates.

The newly infected cell monolayer was then overlaid with 0.5% agarose dissolved in cell growth media with 2% HS, incubated for 3 to 5 days at 37° C. under 5% CO₂, and then stained for about 2 to 3 hours using 150 μL 3-(4,5-dimethyl-2-thiazolyl)-2,5-diphenyl-2H-tetrazolium bromide solution at 5 mg/mL (MTT, Sigma-Aldrich). The live cells of the MTT-stained monolayers turn a blue/black color, while zones of dead cells killed by the virus form plaques that can be counted. Viral plaques were manually counted and a titer was determined for each test compound. Using the titers, an EC₅₀, EC₉₀, and CC₅₀ were calculated for each compound. The EC₅₀ and EC₉₀ are the concentration of compound that inhibits 50% or 90%, respectively, of viral release into the culture medium as compared to an untreated control. The CC₅₀ is a measure of cytotoxicity caused by the test compound (in the absence of viral infection) and equals the concentration that affects the viability of 50% of the treated cells as compared to untreated cells. The data are presented in Table 2.

TABLE 2
Inhibition of Viral Release (MOI = 1)

| Compound | EC₅₀ | | EC₉₀ | | CC₅₀ | | TI** |
|---|---|---|---|---|---|---|---|
| Celgosivir | 2.0 ± 1.3 | μM | 7.6 ± 2.3 | μM | >2000 | μM | >1000 |
| Castanospermine | 19.4 ± 8.3 | μM | 89 ± 21 | μM | >2000 | μM | >100 |
| Interferon-α2b | 8.0 ± 6.8 | IU*/mL | 114 ± 92 | IU/mL | >1000 | IU/mL | >200 |
| Ribavirin | 1.5 ± 1 | μM | 5.7 ± 2.7 | μM | 250 | μM | ˜166 |

*IU = Interferon Units
**TI is the Therapeutic Index (CC₅₀/EC₅₀)

The CC₅₀ results show that all of these test compounds are not cytotoxic near their EC₅₀ or EC₉₀ values and show a very favorable therapeutic index (i.e., not cytotoxic at therapeutically relevant concentrations). The EC₅₀ and EC₉₀ values show that each of the test compounds (celgosivir, castanospermine, interferon, ribavirin) have a direct anti-viral effect, which indicates that HCV would also be directly inhibited by celgosivir, castanospermine, interferon and ribavirin.

### Example 2

### Protection of MDBK Cells from BVDV-Induced Cytopathicity by Test Compounds

Cell proliferation assays were performed using a non-radioactive cell proliferation MTS/PMS assay. MTS is 3-(4,5-dimethylthiazol-2-yl)-5-(3-carboxymethoxy phenyl)-2-(4-sulfophenyl)-2H-tetrazolium (Promega Corporation, Madison, Wis.)) and PMS is phenazine methosulfate (Sigma Aldrich, St. Louis, Mo.). MDBK cells were seeded into 96-well plates at a density of approximately 2×10⁴ cells per well and incubated at 37° C., 5% CO₂ for about 24 hours to allow attachment of the cells to the tissue culture plates prior to infection and treatment with the test compounds. The cell monolayers were infected with sufficient plaque forming units of BVDV diluted in sterile phosphate buffered saline (PBS) containing 1% HS and 1 mM MgCl₂ with (PFU) to achieve a desired MOI (from about 0.001 to about 0.1 virus per cell), incubated at 37° C., 5% CO₂ for about 1 to about 2 hours, and then washed with PBS. The infected and washed cells were suspended in cell growth medium having 2% HS or in cell growth medium having 2% HS containing various concentrations of test compounds. Ujninfected cells were also used as an additional control. The test compounds used included amantadine, celgosivir, castanospermine, NM-107, interferon α-2b, ribavirin, peginterferon α-2a, peginterferon α-2b, N-butyldeoxynojirimycin (NB-DNJ), interferon αcon-1, interferon α-n3, interferon Omega, and non-nucleoside compound (L)-2-[(1-Benzyl-1H-indole-6-carbonyl)-amino]-3-(1H-indol-3-yl)-propionic acid (2-BAIP). The control and treated cells were done in triplicate and incubated at 37° C., 5% CO₂ for about 3 to about 4 days.

After the treatment, the cells were suspended in an MTS/PMS solution at a final concentration of 333 μg/ml MTS and 25 μM PMS, incubated for 1 to 4 hours at 37° C. in a humidified, 5% CO₂ atmosphere, and then the absorbance at 490 nm (OD₄₉₀) was measured on a spectrophotometer plate reader. The mean absorbance for each set of triplicate wells was determined. Antiviral activity (i.e., reduction of BVDV cytopathicity) was measured as MTS conversion relative to the differential between the conversion for non-drug treated cells that were non-infected and infected. The cytopathic effect (CPE) reduction for each concentration of the tested compound, which correlated with antiviral activity, was calculated as follows:
% CPE reduction=[(D−ND)/(NI−ND)]×100,

in which D is the absorbance of drug-treated cells; ND is the absorbance of non drug-treated infected cells; and NI is the absorbance of non-infected cells. From the treated and infected cells, an EC₅₀ was calculated, which represents the concentration of drug that protects 50% of the cells from BVDV-induced cytopathicity (50% CPE reduction). From the non-treated and uninfected cells, a CC₅₀ was calculated, which is a measure of drug cytotoxicity and equals the concentration of drug that affects the viability of 50% of the MDBK cells. The data are presented in Table 3.

TABLE 3
Protection of MDBK Cells from BVDV-Induced Cytopathicity and Drug
Cytotoxicity

| Compound (MOI*) | EC₅₀ | | CC₅₀ | | TI |
|---|---|---|---|---|---|
| Celgosivir (0.01) | 7.7 ± 3 | μM | >300 | μM | >39 |
| Castanospermine (0.01) | 62 ± 15 | μM | >1000 | μM | >16 |
| Interferon-α2b (0.01) | 19.4 ± 6 | IU^{†}/mL | >300 | IU/mL | >15 |
| Ribavirin (0.01) | 4.4 ± 2 | μM | 25 to >60 | μM | 5.7 to >13 |
| Amantadine (0.05) | 476, 357 | μM | >1000 | μM | >2 |
| NB-DNJ^{‡} (0.05) | 459 | μM | >500 | μM | >1 |
| NM-107 (0.01) | 2.1 ± 0.5 | μM | 228 | μM | 100 |
| Peginterferon-α2b (0.01) | 13.1 ± 7.6 | pM | >200 | pM | >15 |
| Peginterferon-α2a (0.01) | 16.6 ± 11 | IU/mL | >1000 | IU/mL | >60 |
| Interferon-αcon-1 (0.01) | 98 ± 22 | IU/mL | >400 | IU/mL | >4 |
| Interferon-α-n3 (0.01) | 27 ± 5 | IU/mL | >1000 | IU/mL | >36 |
| Interferon-ω (0.01) | 37 ± 1.4 | IU/mL | >1000 | IU/mL | >27 |
| 2-BAIP^{∥} (0.05) | >50 | μM | >50 | μM | NA |
| Interferon-γ (0.01) | >1000 | IU/mL | >1000 | IU/mL | NA |
| Interferon-β-1a (0.01) | >1000 | IU/mL | >1000 | IU/mL | NA |

*MOI = multiplicity of infection
^{†}IU = interferon units
^{‡}NB-DNJ = N-butyldeoxynojirimycin
^{∥}(L)-2-[(1-Benzyl-1H-indole-6-carbonyl)-amino]-3-(1H-indol-3-yl)-propionic acid

The CC₅₀ results show that all of these test compounds are not cytotoxic near their EC₅₀ values with TIs (Therapeutic Indexes) greater than about 10 (i.e., unlikely to be cytotoxic at therapeutically relevant concentrations), except maybe for NB-DNJ and possibly amantidine. The EC₅₀ values show that at least three compounds-2-BAIP, interferon-y and interferon-β-1a- do not protect MDBK cells from BVDV-induced cytopathicity at the concentrations tested. The rest of the listed compounds can protect cells from virally-induced cytopathicity, which indicates that HCV would be directly inhibited by compounds such as celgosivir, castanospermine, interferon-α2b, peginterferon-α2b, peginterferon-α2a, NM-107, ribavirin and others.

### Example 3

### Synergy of Castanospermine or Celgosivir in Combination with Other Drugs Chechboard Approach

A double combination assay was performed using MDBK cells infected with BVDV in an inhibition of cytopathic effect (CPE) assay as described in Example 2. The double drug combinations were measured by creating a “checkerboard” of drug concentrations used on cell monolayers in microtiter plates, with one drug being titrated horizontally and the other drug titrated vertically, and each double combination being tested at least twice. The combined drug efficacy data were analyzed using a MacSynergy™ II software program (gift from Dr. Mark Prichard, University of Alabama, Tuscaloosa, Ala.) to determine whether the combinations showed synergistic activity (see, e.g., Ouzounov et al., supra; Buckwold et al., Antimicrob. Agents Chemother. 47:2293, 2003).

TABLE 4
Concentration Ranges Used for the Double Combination Treatment

| Combination | | Range Tested | | |
|---|---|---|---|---|
| Compound 1 | Compound 2 | Compound 1 | Compound 2 | |
| Celgosivir | Interferon-α2b | 20-0.3 μM | 60-0.7 | IU/mL |
| Celgosivir | Ribavirin | 20-0.3 μM | 20-0.3 | μM |
| Celgosivir | NM-107 | 60-0.7 μM | 20-0.3 | μM |
| Celgosivir | Amantadine | 20-0.3 μM | 500-31.3 | μM |
| Celgosivir | NB-DNJ | 20-0.3 μM | 500-6.2 | μM |
| Celgosivir | 2-BAIP | 20-0.3 μM | 50-3.1 | μM |
| Castanospermine | Interferon-α2b | 300-1.2 μM | 250-0.1 | IU/mL |
| Catsanospermine | Ribavirin | 300-3.7 μM | 30-0.3 | μM |
| Catsanospermine | NM-107 | 100-1.2 μM | 20-0.3 | μM |
| Catsanospermine | Amantadine | 33-1.2 μM | 500-31.3 | μM |
| Catsanospermine | NB-DNJ | 33-1.2 μM | 500-31.3 | μM |
| Catsanospermine | 2-BAIP | 33-1.2 μM | 50-3.1 | μM |
| Ribavirin | Interferon-α2b | 30-0.3 μM | 250-0.1 | IU/mL |

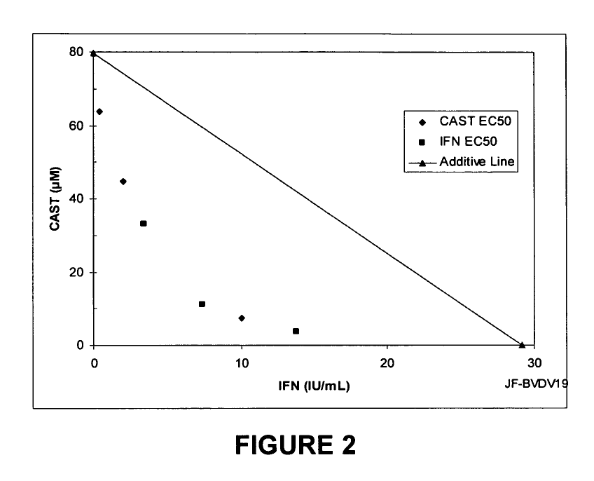

The inhibition of cytopathic effect (CPE) for each drug is provided as an EC₅₀, which represents the concentration of test compound that provides 50% protection of BVDV-induced cytopathicity. The EC₅₀ values of a first test compound derived while in combination with a second test compound were plotted against the corresponding concentration of the second test compound to create an isobole (dose pair). All of the isoboles were plotted in an isobologram to determine the presence of synergy, antagonism or additivity for the combined test compounds. A straight line was plotted between the monotherapy EC₅₀ values of each of the two test compounds (e.g., castanospermine and interferon, or castanospermine and ribavirin, or celgosivir and NM-107). The line connecting the monotherapy EC₅₀ values represents the theoretical additivity effect values for the two compounds. Isoboles of combination treatments that plot below the additivity line indicate synergy when the two test compounds are combined (i.e., the combination shows better activity than the compounds have individually), while isoboles above the additivity line indicate antagonism (i.e., the combination shows less activity than the compounds have individually). See FIGS. 2, 4, 6, 8, and 13.

In addition to generating isobolograms, the checkerboard data was imported into MacSynergy™ II software to graph the observed synergy (or additive or antagonism) volumes for the double combinations tested. Briefly, the calculated additive interactions were subtracted from the experimentally determined values to reveal the corresponding drug concentrations at which a synergistic (indicated by positive % values) or antagonistic (indicated by negative % values) effect is observed. The greater the positive percent volume observed, the greater the synergy between the two compounds. More specifically, values less than about 25 μM²% or μM(IU/ml) % are considered insignificant; values between about 25-50 μM²% or μM(IU/ml) % are considered minor but significant; values between about 50-100 μM²% or PM(IU/ml) % are considered indicative of moderate synergy (which may be indicative of a significant synergistic effect in vivo); and values greater than about 100 μM²% or μM(IU/ml) % are considered indicative of strong synergy (which is likely indicative of a significant synergistic effect in vivo). In contrast, any value about or less than −25 μM²% or μM(IU/mL) % is indicative of a significant antagonistic effect. The data presented in Table 5 represent volumes of synergy or antagonism with 95% confidence. The confidence level was calculated using a Bonferroni adjustment as a conservative estimate of significance to statistically evaluate the data.

TABLE 5
Efficacy Volumes of Double Combination Treatment of BVDV Infected Cells

| | Efficacy Volume (95% Confidence) | | | |
|---|---|---|---|---|
| Combination (MOI) | Synergy | | Antagonism | |
| Castanospermine + Interferon-α2b (0.01) | 231 ± 53 | μM(IU/mL)% | −2 ± 2 | μM(IU/mL)% |
| Catsanospermine + Ribavirin (0.01) | 97 ± 11 | μM²% | −145 ± 9 | μM²% |
| Castanospermine + NM-107 (0.05) | 93 | μM²% | −33 | μM²% |
| Castanospermine + Amantadine (0.05) | 61 | μM²% | −1.5 | μM²% |
| Castanospermine + NB-DNJ (0.05) | 16 | μM²% | −8.5 | μM²% |
| Castanospermine + 2-BAIP (0.05) | 6 | μM²% | −9.3 | μM²% |
| Celgosivir + Interferon α2b (0.01) | 148 ± 35 | μM(IU/mL)% | −2 ± 3 | μM(IU/mL)% |
| Celgosivir + Ribavirin (0.01) | 45 ± 26 | μM²% | −163 ± 124 | μM²% |
| Celgosivir + NM-107 (0.01) | 132 | μM²% | −50 | μM²% |
| Celgosivir + Amantadine (0.05) | 138 | μM²% | −69 | μM²% |
| Celgosivir + NB-DNJ (0.05) | 172 | μM²% | −4.7 | μM²% |
| Celgosivir + 2-BAIP (0.05) | 25 | μM²% | −85 | μM²% |
| Ribavirin + IFN-α2b (0.01) | 68 ± 1 | μM(IU/mL)% | −102 ± 9 | μM(IU/mL)% |

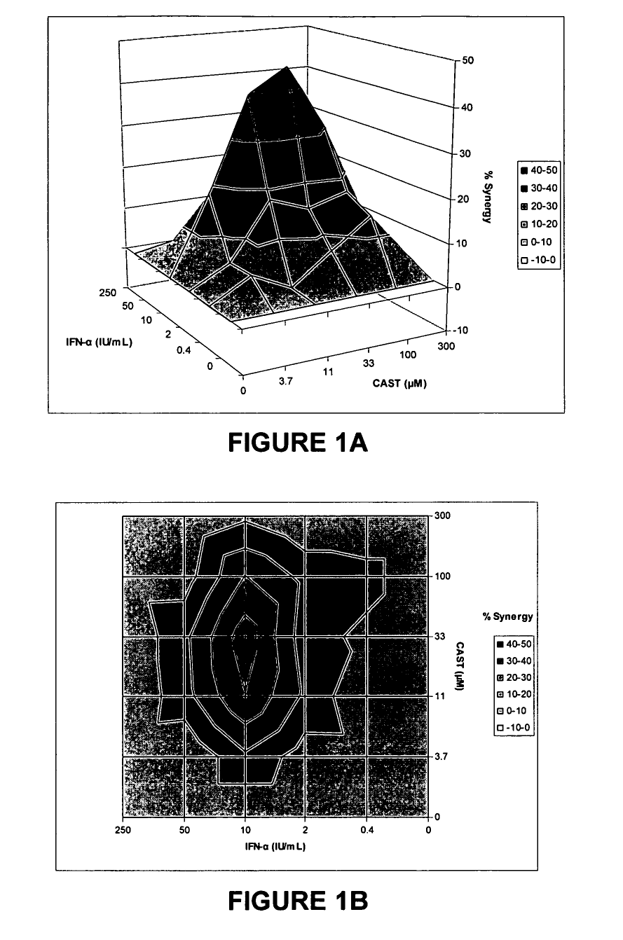

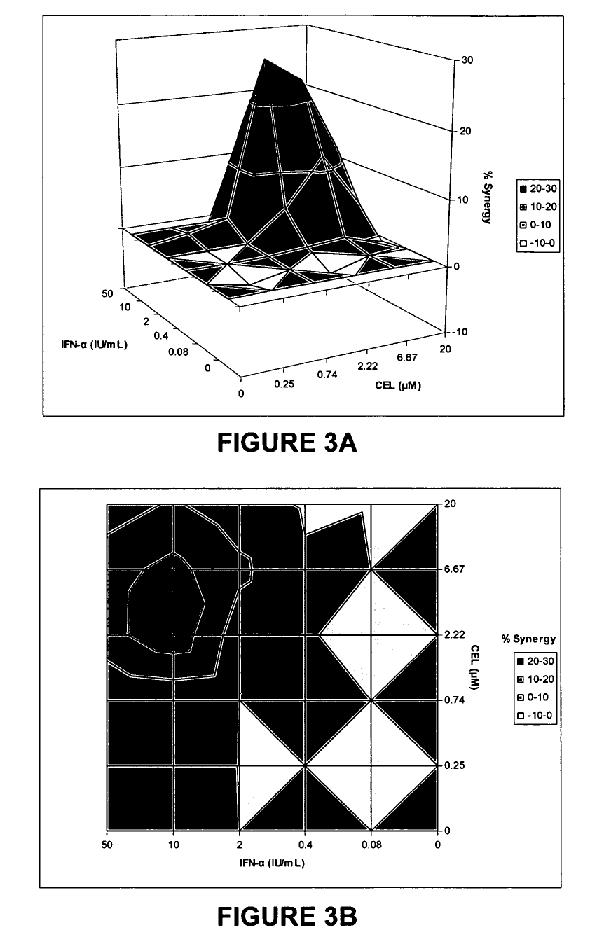

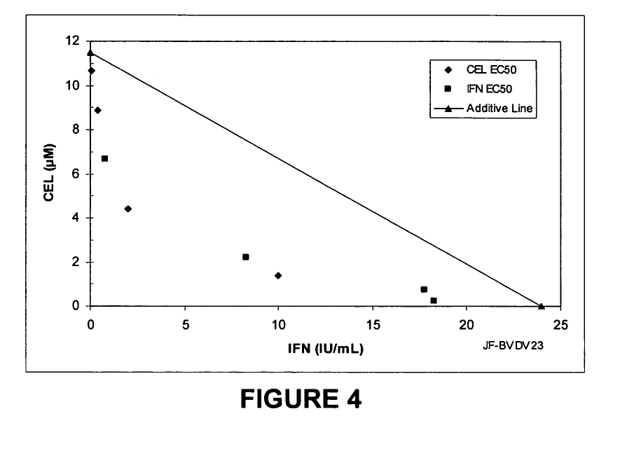

The combination of castanospermine or celgosivir with interferon-α2b demonstrated strong synergy in efficacy against BVDV-infected MDBK cells (Table 5, rows 1 and 7, respectively), and no significant antagonistic effects (i.e., all values were between 0 and −25 μM(IU/mL) %), at all combination of concentrations tested. Synergism peaks were located at castanospermine or celgosivir concentrations between 25 μM and 33 μM and an interferon-α2b concentration of 10 IU/mL (see FIGS. 1 and 3, respectively). Analysis of the combination data using an isobologram confirms the strong synergy observed for the combination of castanospermine or celgosivir with interferon-α2b. The synergy observed between celgosivir and interferon is consistent with what was known in the art (see, e.g., U.S. Patent Publication No. 2004/0147549, July 29, 2004). For example, at 10 IU/mL interferon-α2b, the EC₅₀ of castanospermine is reduced by more than 7-fold, while a less than a 2-fold reduction was expected if the interaction was only additive (see FIGS. 2 and 4, respectively).

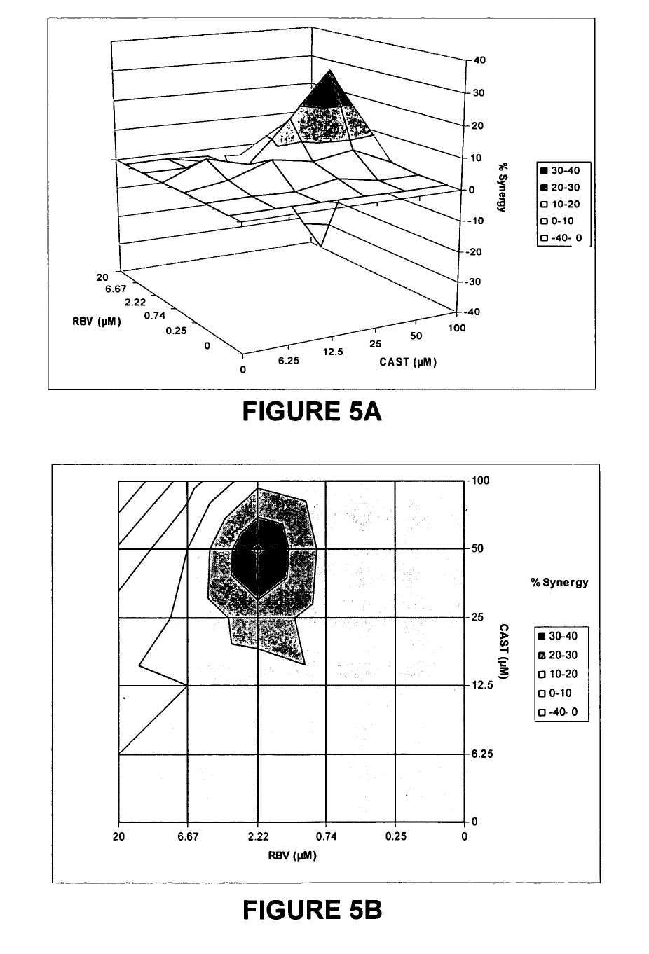

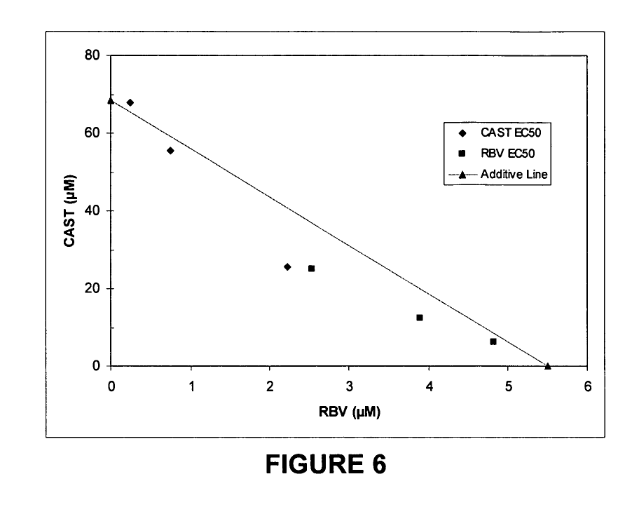

The combination of castanospermine with ribavirin combination demonstrated moderate synergy in efficacy against BVDV-infected MDBK cells (Table 5, row 2). Synergism peaks were located at castanospermine concentrations between 10 μM and 50 μM and ribavirin concentrations between 1 μM and 6 μM, with the maximum percent synergy reached at between 22% and 31% (see FIG. 5). Antagonistic effects in efficacy were observed at very high concentrations of the compounds (see FIG. 5)—for example, antagonistic peaks occurred at a castanospermine concentration of 300 μM and a ribavirin concentration of 30 μM, which are unlikely to be relevant in vivo (i.e., therapeutically). The maximum percent antagonism reached was approximately −40%. The isobologram of the combination of castanospermine with ribavirin shows that there is a moderate synergistic interaction between these compounds. For example, at about 2 μM ribavirin, the EC₅₀ of castanospermine is reduced by about 2- to 3-fold, while a less than 2-fold reduction was expected if the interaction was only additive (see FIG. 6).

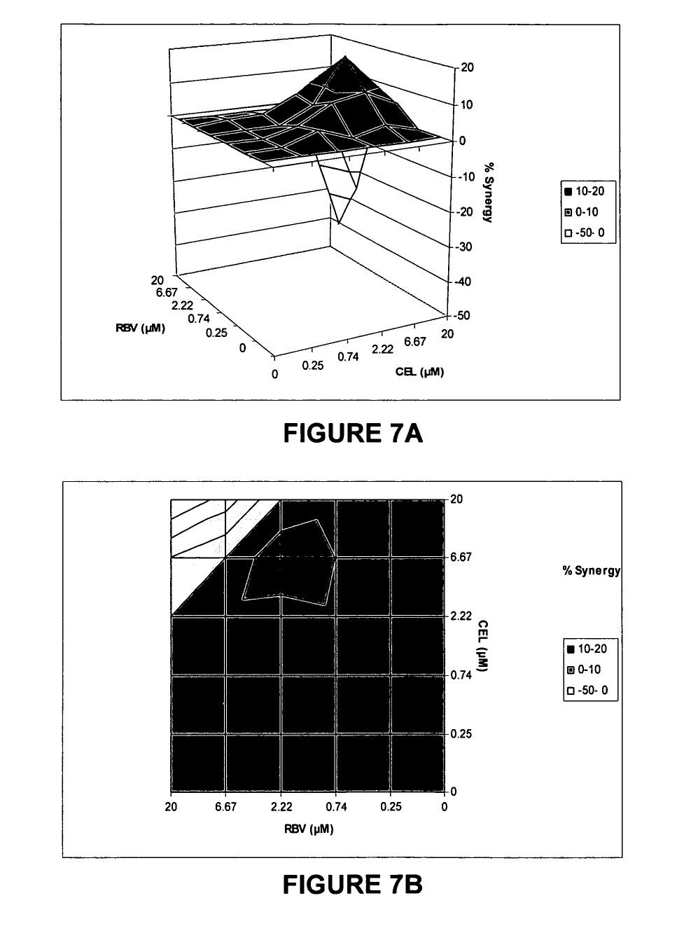

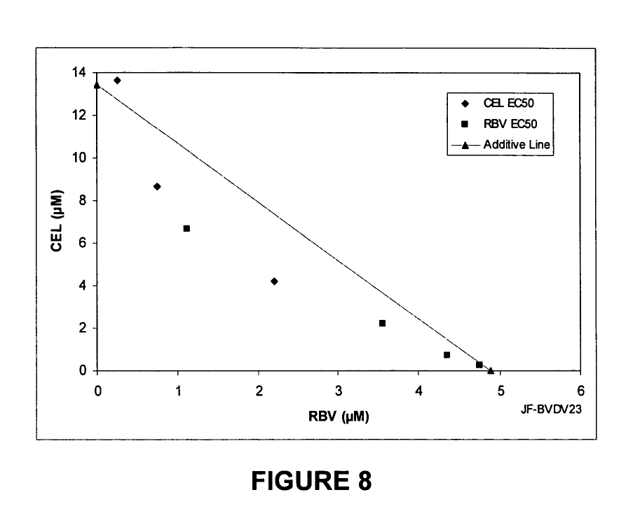

The combination of celgosivir with ribavirin demonstrated moderate synergy in efficacy against BVDV-infected MDBK cells (Table 5, row 8). Antagonistic effects in efficacy were observed at very high concentrations of the compounds—for example, antagonistic peaks occurred at a celgosivir concentration of 20 μM and a ribavirin concentration of 20 μM (see FIG. 7), which are unlikely to be relevant in vivo (i.e., therapeutically). The isobologram of the combination of celgosivir with ribavirin indicates moderate synergistic interaction between these compounds. For example, at a concentration of 2 μM ribavirin, the EC₅₀ of celgosivir is reduced by 3-fold, while about only about a 2-fold reduction was expected if the interaction was only additive (see FIG. 8).

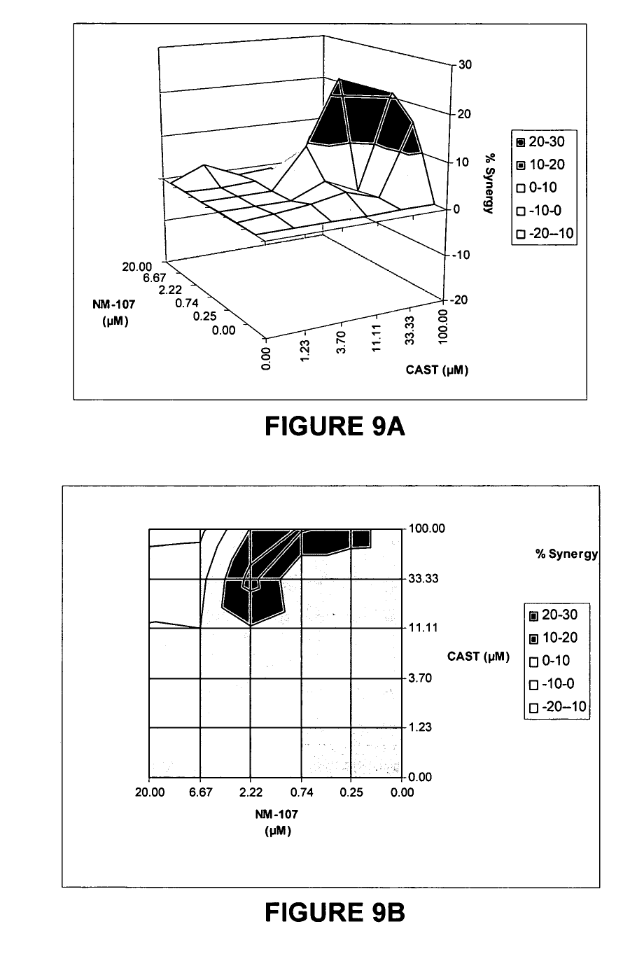

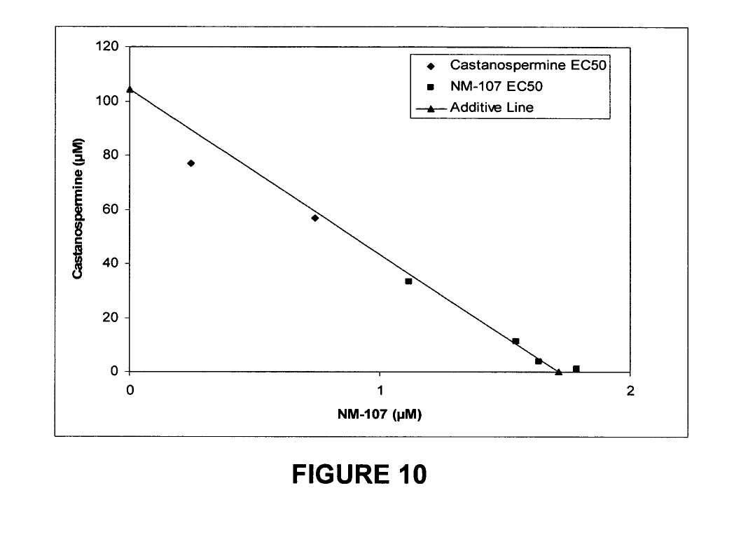

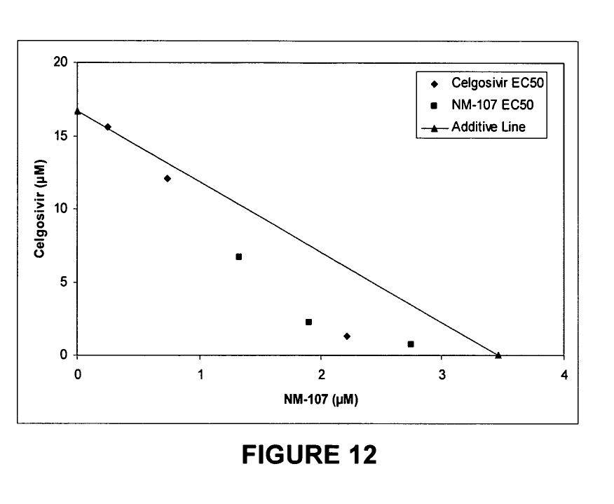

The combination of castanospermine with NM-107 demonstrated only moderate synergy, while the combination of celgosivir with NM-107 demonstrated strong synergy in efficacy against BVDV-infected MDBK cells (see Table 5, rows 3 and 9, respectively). Moderate antagonistic effects in efficacy for the combination of castanospermine or celgosivir with NM-107 began to appear at the higher concentrations of the two drugs (Table 4, rows 3 and 9, respectively). Antagonistic peaks began to appear when the NM-107 concentration was at greater than about 20 μM and castanospermine was at greater than about 100 μM or celgosivir was at greater than about 60 μM and (see FIGS. 9 and 11). An analysis of the combination of castanospermine with NM-107 using an isobologram reveals that the interaction between these drugs is possibly additive to only slightly synergistic (see FIG. 10). In contrast, analysis of the combination data using an isobologram confirms the strong synergy observed for the combination of celgosivir with NM-107. For example, at 2.2 μM NM-107, the EC₅₀ of celgosivir is reduced by more than about 8-fold, while about a 3-fold reduction was expected if the interaction was only additive (see FIG. 12).

The combination of castanospermine with amantadine or NB-DNJ demonstrated moderate and no significant synergy, respectively, in efficacy against BVDV-infected MDBK cells (see Table 5, rows 4 and 5, respectively). No significant antagonistic effects were observed when castanospermine was combined with amantadine or NB-DNJ at any combination of concentrations tested (see Table 5, rows 4 and 5, respectively). By contrast, the combination of celgosivir with amantadine or NB-DNJ demonstrated strong synergy in efficacy against BVDV-infected MDBK cells (see Table 5, rows 10 and 11, respectively). Higher concentrations of celgosivir and amantadine began to show a moderate antagonistic interaction (celgosivir at a concentration of greater than about 20 μM and amantadine at greater than about 500 μM; data not shown and Table 4, row 10). No significant antagonistic effects were observed, however, when celgosivir and NB-DNJ were combined at any combination of concentrations tested (see Table 5, row 11). Finally, the combination of castanospermine or celgosivir with the non-nucleoside inhibitor 2-BAIP demonstrated no significant synergy (see Table 5, rows 6 and 12, respectively). The combination of castanospermine with 2-BAIP demonstrated no significant antagonistic effects (see Table 5, row 6), while the combination of celgosivir with 2-BAIP showed a moderate antagonism (see Table 5, row 12).

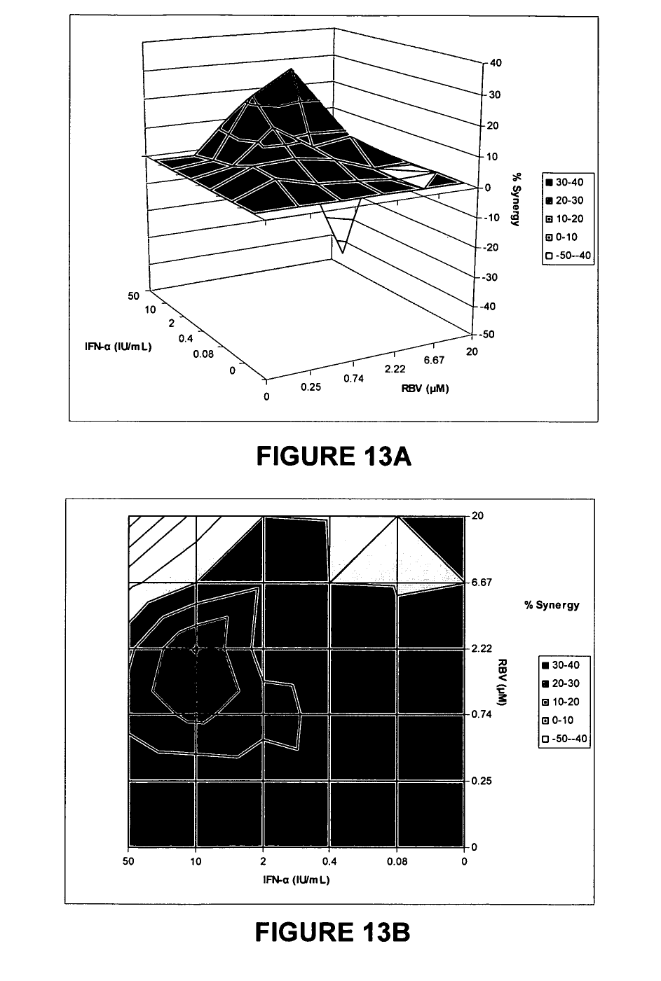

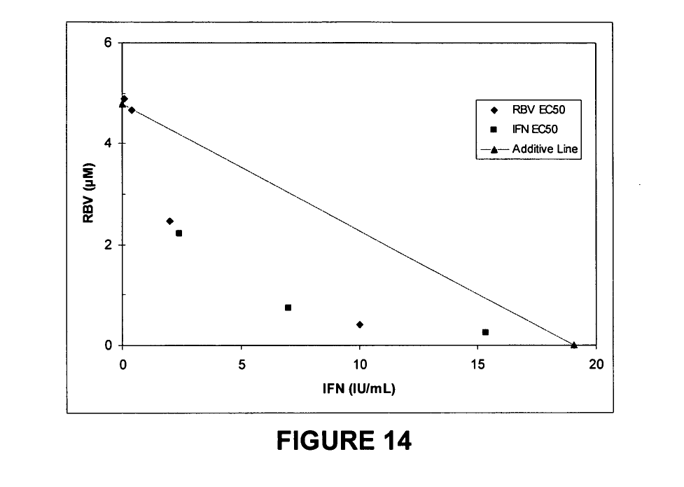

The combination of interferon-α2b with ribavirin demonstrated moderate synergy in efficacy against BVDV-infected MDBK cells (Table 5, row 13). A similar volume of synergy has been reported in literature by Buckwold et al., 2003, and discussed herein. Antagonistic effects in efficacy were also observed at high concentrations of drugs, with antagonistic peaks occurring for interferon-α2b at concentrations of greater than about 50 IU/mL and for ribavirin at concentrations of greater than about 20 μM (FIG. 13). An isobologram derived from the combination of interferon-α2b with ribavirin further confirms that there is synergy between interferon-α2b and ribavirin. For example, at about 10 IU/mL interferon-α2b, the EC₅₀ of ribavirin is reduced by up to about 6-fold, while about a 2-fold reduction was expected if the interaction were additive (FIG. 14).

In sum, double combinations with celgosivir tend to show strong synergistic interactions (volumes of synergy greater than about 100 (IU/mL)μM %), while the double combinations with castanospermine tend to show more moderate synergy (between 25 and 100 (IU/mL)μM %). Thus, a variety of new and known double combinations with castanospermine and derivatives thereof, such as celgosivir, are unexpectedly more efficacious against Flaviviridae infections than the efficacy of the compounds on an individual basis.

### Example 4

### Synergy of Castanospermine or Celgosivir in Combiniation with Interferon Fixed Ratio Approach

The inhibition-of-cytopathic effect (CPE) assay of Example 2 was used to analyze the interaction of celgosivir or castanospermine combined with Interferon α-2b (PBL Biomedical Laboratories, Piscataway, N.J.), Peginterferon α-2a (International Rx Specialty Company, Bastrop, Tex.), Peginterferon α-2b (International Rx Specialty Company, Bastrop, Tex.), Interferon λ (PeproTech, Rocky Hill, N.J.), Interferon α con-1 (International Rx Specialty Company, Bastrop, Tex.), Interferon α-n3 (International Rx Specialty Company, Bastrop, Tex.), or Interferon co (Cedarlane Laboratories, Hornby, ON). The compounds were combined at fixed molar ratios and serially diluted 2-fold in cell growth medium to examine a range of 6 fixed ratio combinations including those having about an equipotent antiviral dose to a combination in which one test compound was used at a sub-optimal (e.g., sub-therapeutic) level. The corresponding monotherapies were conducted in parallel to these combination treatments (EC₅₀ values for the monotherapy treatments are provided in Table 3).

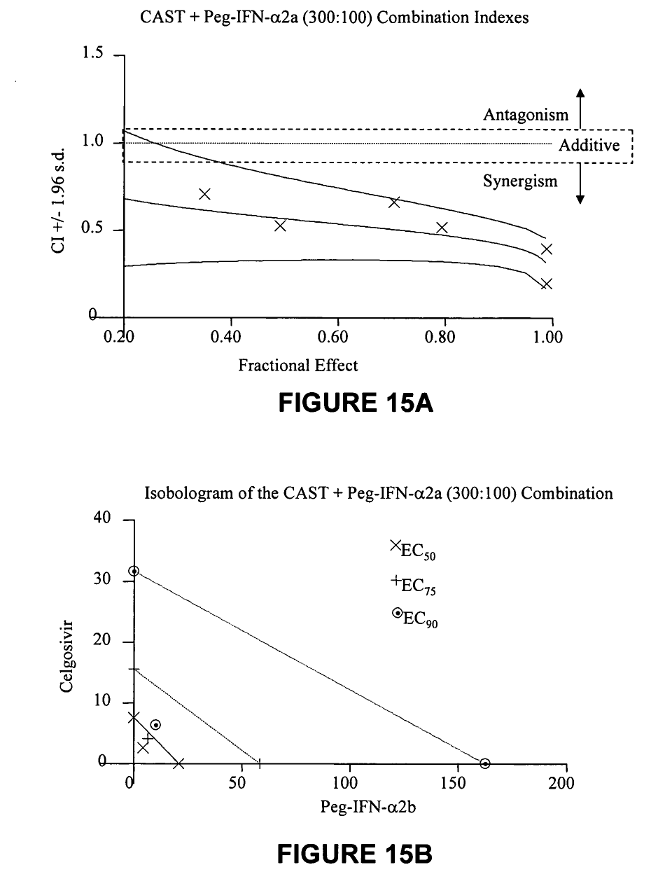

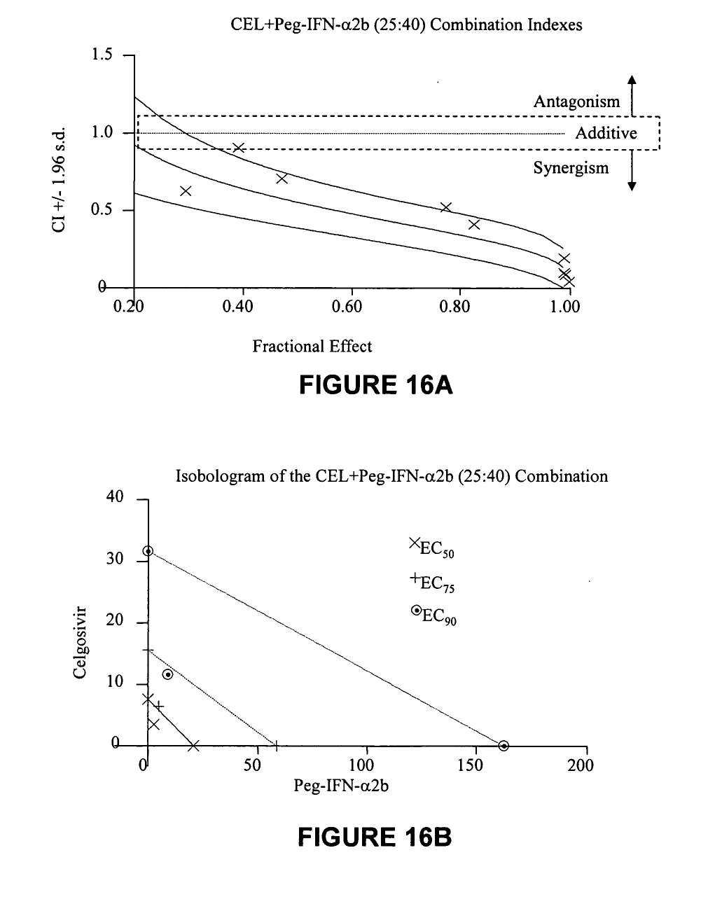

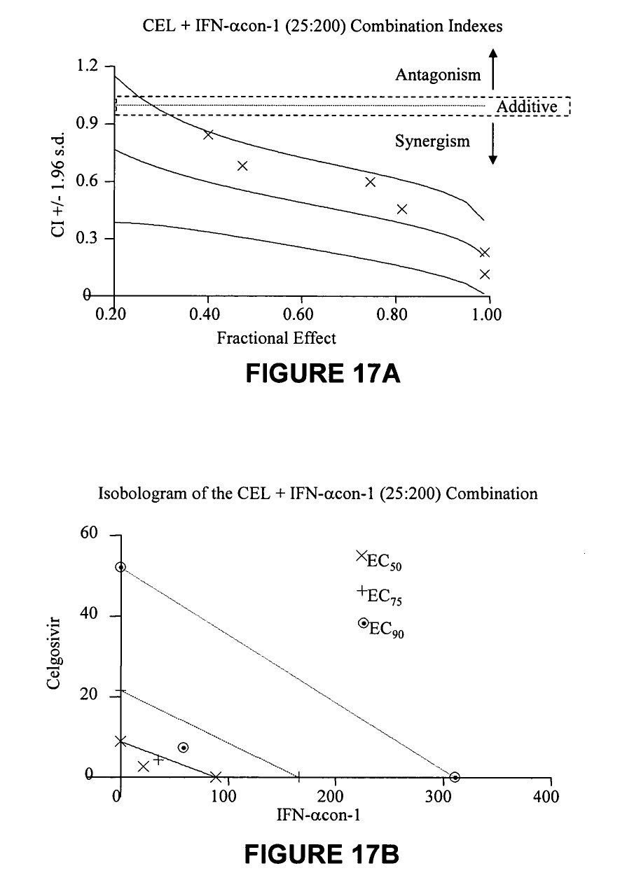

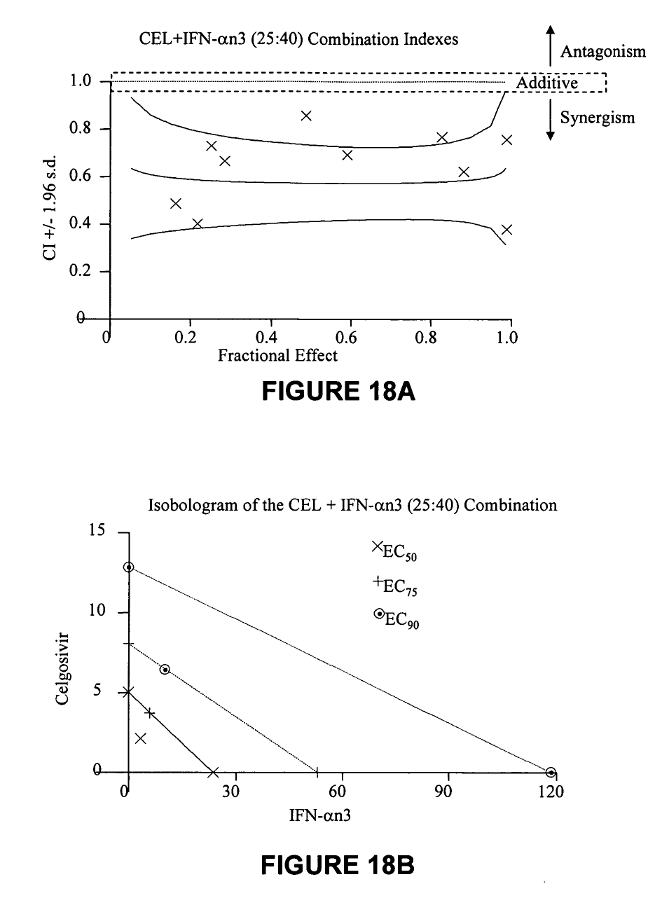

The protection against BVDV-induced cytopathic effect in MDBK cells (MOI of 0.01) by the combined test compound treatments was quantified and the test compound interactions (synergism, additivity or antagonism) were analyzed with the CalcuSyn™ program (Version 2.0, Biosoft, Inc., UK) to generate a Combination Index (CI) value, in which a CI value of 1 equals additivity. The following criteria were used: CI values above 1.45 indicate strong antagonism; CI values between 1.2 and 1.45 indicate moderate antagonism; values between 1.10 and 1.20 indicate slight antagonism; values between 0.90 and 1.10 are nearly additive; values between 0.85 and 0.90 indicate slight synergism; values between 0.7 and 0.85 indicate moderate synergism; values between 0.30 and 0.70 indicate good synergism; values between 0.10 and 0.30 indicate strong synergism; and values below 0.10 indicate very strong synergism. These values are plotted in Fraction of virus affected versus Combination Index plots (Fa-Cl plots), which are generally the most useful in determining drug interactions because the Monte Carlo analysis provides a measure of statistical significance (i.e., these plots have three lines, which represent the median value (middle line) and ±1.96 standard deviations (upper and lower lines)). See, for example, FIGS. 15-18.

In addition, isobolograms were generated, which provide an excellent secondary measure of the drug combination interactions. For these plots, EC₅₀, EC₇₅, and EC₉₀ values for the combination treatments are displayed as single points. Values that fall to the right of (above) the additivity line (i.e., the line drawn between the EC value for each drug as a monotherapy) indicate antagonism, values to the left of (below) the additivity line indicate synergy, and values on or near the line indicate additivity.

TABLE 6
Combination Indexes (CIs) of Various Celgosivir or Castanospermine Double
Combinations

| Compound | Ratio | CI (EC₅₀) | CI (EC₇₅) | CI (EC₉₀) |
|---|---|---|---|---|
| Celgosivir + Interferon-α-con-1 | 25:200 | 0.54 | 0.42 | 0.33 |
| Celgosivir + Interferon-α-con-1 | 25:400 | 0.66 | 0.53 | 0.43 |
| Celgosivir + Interferon-α-n3 | 25:40 | 0.57 | 0.57 | 0.59 |
| Celgosivir + Interferon-α-n3 | 25:80 | 0.82 | 0.72 | 0.64 |
| Celgosivir + Interferon-λ1 | 20:250 | 0.99 | 1.23 | 1.53 |
| Celgosivir + Interferon-λ1 | 25:400 | 0.61 | 0.58 | 0.57 |
| Celgosivir + Interferon-λ1 | 25:800 | 0.70 | 0.62 | 0.57 |
| Celgosivir + Interferon-ω | 25:1200 | 0.70 | 0.65 | 0.61 |
| Celgosivir + Interferon-ω | 25:600 | 0.54 | 0.55 | 0.57 |
| Celgosivir + Peg-Interferon-α2a | 25:20 | 0.87 | 0.86 | 0.85 |
| Celgosivir + Peg-Interferon-α2a | 25:100 | 0.94 | 0.67 | 0.49 |
| Celgosivir + Peg-Interferon-α2a | 25:40 | 0.56 | 0.42 | 0.32 |
| Celgosivir + Peg-Interferon-α2a | 20:200 | 1.00 | 0.84 | 0.70 |
| Celgosivir + Peg-Interferon-α2b | 25:20 | 0.60 | 0.50 | 0.42 |
| Celgosivir + Peg-Interferon-α2b | 25:20 | 0.70 | 0.59 | 0.52 |
| Celgosivir + Peg-Interferon-α2b | 25:40 | 0.55 | 0.38 | 0.26 |
| Celgosivir + Peg-Interferon-α2b | 25:40 | 0.69 | 0.58 | 0.50 |
| Celgosivir + Interferon-α2b | 25:30 | 0.56 | 0.56 | 0.56 |
| Celgosivir + Interferon-α2b | 25:60 | 0.67 | 0.67 | 0.68 |
| Castanospermine + Peg-Interferon-α2a | 300:100 | 0.57 | 0.49 | 0.43 |
| Castanospermine + Peg-Interferon-α2a | 300:200 | 0.71 | 0.64 | 0.57 |

As is evident from Table 6 and FIGS. 15-18, the combination of celgosivir with a variety of different interferons (type I and others) showed measurable synergy at most ratios.

### Example 5

### Synergy of Castanospermine or Celgosivir in Combination with NM-107 and Ribavirin Fixed Ratio Approach

The inhibition of cytopathic effect (CPE) assay of Example 2 was used to analyze the interaction of celgosivir or castanospermine combined with NM-107 (Toronto Research Chemicals, Canada) or ribavirin (Sigma-Aldrich). Testing and analysis was performed as described in Example 4.

TABLE 7
Combination Index of Celgosivir Combined with NM-107 or Ribavirin

| Compound | Ratio (μM) | CI (EC₅₀) | CI (EC₇₅) | CI (EC₉₀) |
|---|---|---|---|---|
| Celgosivir + NM-107 | 20:2.22 | 0.81 | 0.83 | 0.86 |
| Celgosivir + NM-107 | 20:6.67 | 0.55 | 0.59 | 0.62 |
| Celgosivir + NM-107 | 25:10 | 1.01 | 0.86 | 0.73 |
| Celgosivir + NM-107 | 25:5 | 1.14 | 1.05 | 0.97 |
| Celgosivir + Ribavirin | 25:3 | 0.93 | 0.81 | 0.73 |
| Celgosivir + Ribavirin | 25:6 | 1.00 | 1.00 | 1.06 |

The combination of celgosivir with NM-107 showed the best synergy when NM-107 was present at more than about 5 μM., while the combination with ribavirin showed slight synergy to additivity with celgosivir.

### Example 6

### Synergy of Castanospermine or Celgosivir in Triple Combinations Checkerboard Approach

The inhibition of cytopathic effect (CPE) assay of Example 2 was used to analyze the interaction of celgosivir or castanospermine combined with interferon-α2b in presence of increasing concentrations of ribavirin (from 0 to about 3.3 μM). Each double or triple combination was performed twice. The combination efficacy data were analyzed using the MacSynergy™ II software program as described herein.

TABLE 8
Synergy Volume of Triple Combinations of Celgosivir or Castanospermine
with Interferon α and Ribavirin.

Synergy Volume (μM(IU/mL)%)*

| | Celgosivir + | Castanospermine + |
|---|---|---|
| Ribavirin (μM) | Interferon-α2b | Interferon-α2b |
| 0 | 96 ± 30 | 168 ± 77 |
| 0.37 | 213 ± 9 | 145 ± 25 |
| 1.1 | 424 ± 124 | 336 ± 142 |
| 3.3 | 460 ± 110 | 624 ± 33 |

*Synergy volumes are with 95% confidence levels as determined by the MacSynergy ™ II software. Data expressed as mean ± standard deviation. Castanospermine concentration range tested was 0-100 μM; Celgosivir concentration range tested was 0-20 μM; and Interferon-α2b concentration range tested = 0-60 IU/mL.

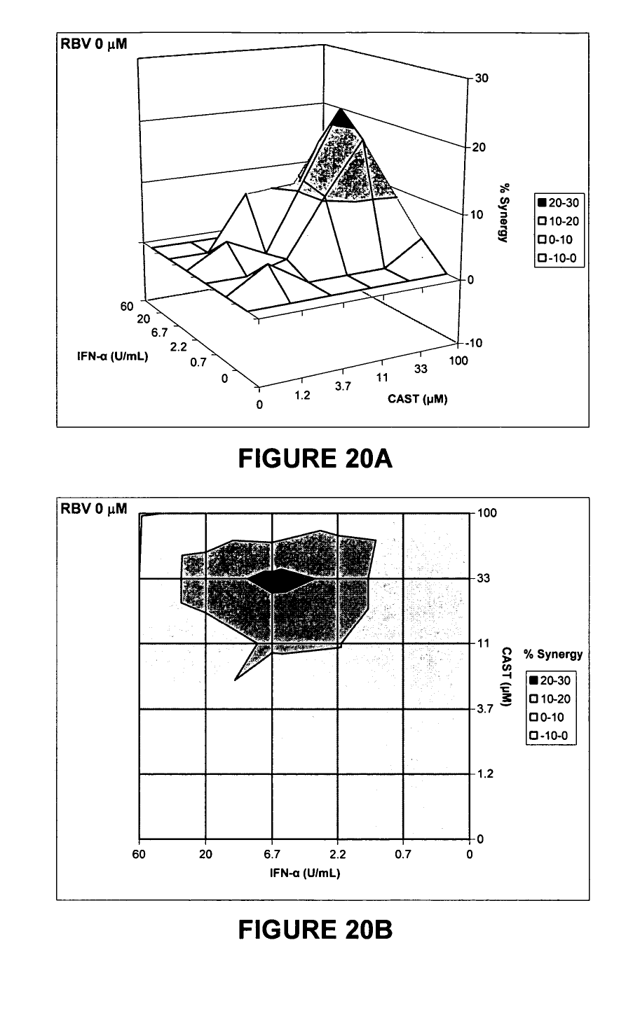

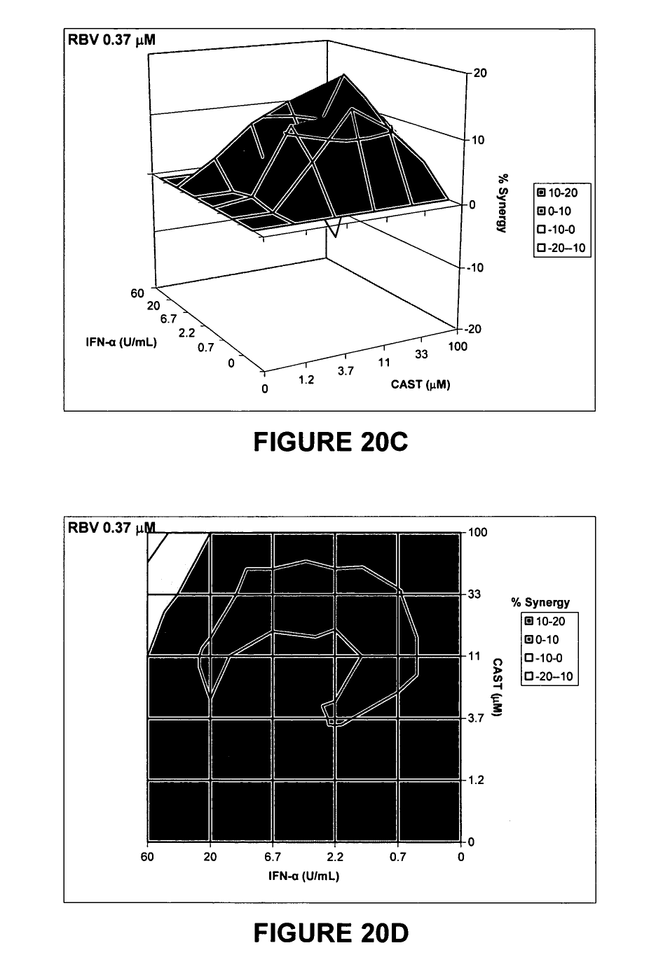

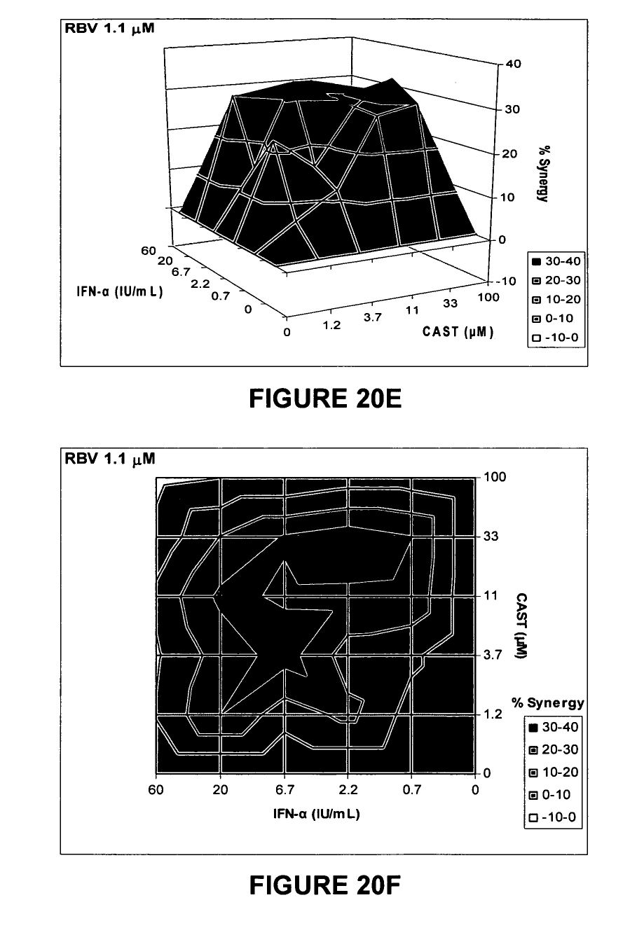

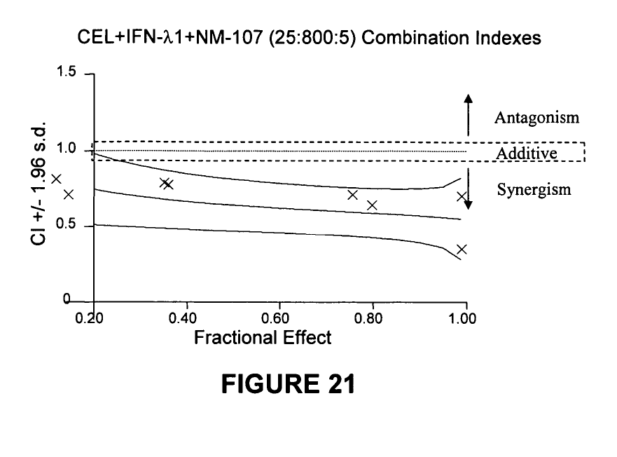

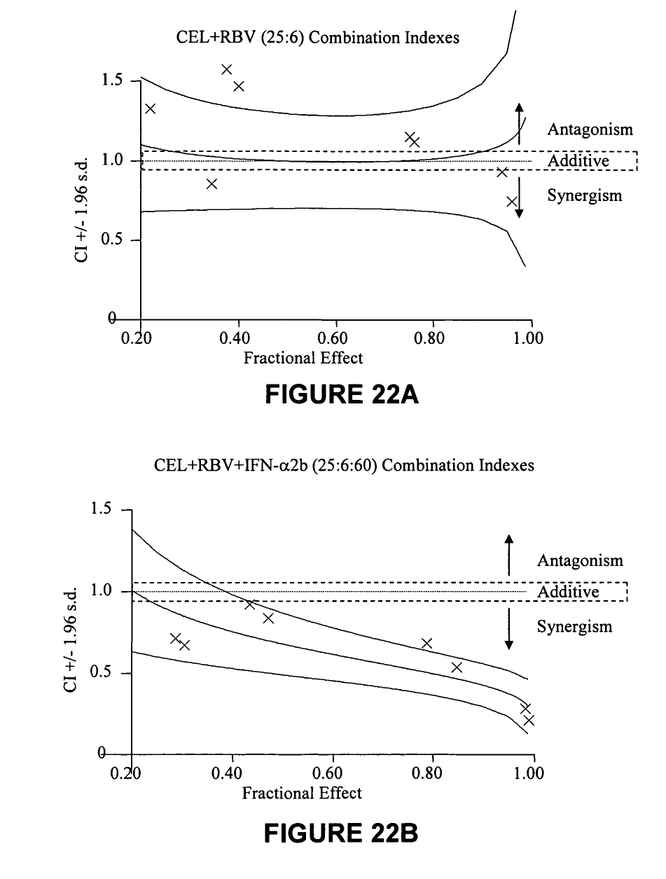

The synergy volumes for the various triple combinations are presented in Table 8. The antiviral activity of celgosivir or castanospermine in a triple combination with interferon-α2b and ribavirin produced strong synergistic effects. At therapeutically relevant concentrations of ribavirin (0.12-3.3 μM), a triple combination with castanospermine or celgosivir and interferon-α2b showed concentration-dependent increases in synergy volume (see Table 8 and FIGS. 19A-F). In comparison to the double combination of interferon-α2b with ribavirin (see FIG. 13), all celgosivir and most castanospermine triple combinations achieved higher synergy volumes and higher peak synergies (see FIGS. 19 and 20).

TABLE 9
Antagonism Volume of Triple Combinations of Celgosivir or
Castanospermine with Interferon α and Ribavirin.

Antagonism Volume
(μM(IU/mL)%)*

| | Celgosivir + | Castanospermine + |
|---|---|---|
| Ribavirin (μM) | Interferon-α2b | Interfron-α2b |
| 0 | −7 ± 3 | 0 ± 1 |
| 0.37 | −4 ± 4 | −12 ± 11 |
| 1.1 | −27 ± 1 | −18 ± 22 |
| 3.3 | −208 ± 118 | −120 ± 14 |

*Antagonism volumes are with 95% confidence levels as determined by the MacSynergy ™ II software. Data expressed as mean ± standard deviation. Castanospermine concentration range tested was 0-100 μM; Celgosivir concentration range tested was 0-20 μM; and Interferon-α2b concentration range tested = 0-60 IU/mL.

In addition, antagonism levels were insignificant or very low in the combination of celgosivir or castanospermine with interferon-α2b when ribavirin doses were between 0 and 1.1 μM (see Table 9). At highest dose of ribavirin (3.3 μM) resulted in a strong level of antagonism for both the celgosivir/interferon-α2b/ribavirin triple combination and the castanospermine/interferon-α2b/ribavirin triple combination (see Table 9). This antagonism was observed at concentrations of greater than about 20 IU/mL of interferon-α2b and greater than about 6.7 μM celgosivir (data not shown). The antagonism observed in presence of 3.3 μM ribavirin is likely due to the cytotoxic effect of ribavirin at this concentration and, thus, reducing the ability of the triple combination from inhibiting cytopathic effect of BVDV.

### Example 7

### Sumergu pf Castanospermine or Celgosivir in Other Triple Combinations Fixed Ratio Approach

The inhibition of cytopathic effect (CPE) assay of Example 2 was used to analyze the interaction of celgosivir or castanospermine in combination with at least two additional test compounds, including various interferons and viral replication inhibitors. The compounds were combined at fixed molar ratios and serially diluted 2-fold in cell growth medium to examine a range of 6 fixed ratio combinations as described in Example 4. The combination efficacy data were analyzed using the CalcuSyn™ II software program as described herein. In addition, isobolograms were generated as a secondary measure of the combined drug interactions. The combination indexes of the triple combinations are compiled in Table 10.

TABLE 10
Combination Index (CI) of Various Celgosivir or Castanospermine Triple Combinations

| Combination | Ratio | CI (EC₅₀) | CI (EC₇₅) | CI (EC₉₀) |
|---|---|---|---|---|
| Celgosivir + IFN-α-con-1 + NM-107 | 25:400:5 | 0.73 | 0.61 | 0.53 |
| Celgosivir + IFN-α-con-1 + NM-107 | 25:200:2.5 | 0.65 | 0.55 | 0.48 |
| Celgosivir + IFN-α-n3 + NM-107 | 25:80:5 | 0.72 | 0.67 | 0.64 |
| Celgosivir + IFN-α-n3 + NM-107 | 25:40:2.5 | 0.68 | 0.67 | 0.66 |
| Celgosivir + IFN-λ1 + NM-107 | 25:800:5 | 0.64 | 0.60 | 0.57 |
| Celgosivir + IFN-λ1 + NM-107 | 25:400:2.5 | 0.63 | 0.62 | 0.62 |
| Celgosivir + IFN-ω + NM-107 | 25:600:2.5 | 0.62 | 0.58 | 0.55 |
| Celgosivir + IFN-ω + NM-107 | 25:1200:5 | 0.71 | 0.71 | 0.73 |
| Celgosivir + NM-107 + IFN-α2b | 20:2.22:20 | 0.71 | 0.70 | 0.70 |
| Celgosivir + NM-107 + IFN-α2b | 25:5:30 | 0.96 | 0.78 | 0.65 |
| Celgosivir + NM-107 + IFN-α2b | 20:6.67:20 | 0.61 | 0.52 | 0.45 |
| Celgosivir + NM-107 + IFN-α2b | 25:10:60 | 0.94 | 0.73 | 0.57 |
| Celgosivir + Peg-IFN-α2a + NM-107 | 25:40:10 | 0.81 | 0.67 | 0.56 |
| Celgosivir + Peg-IFN-α2a + NM-107 | 25:100:5 | 1.03 | 0.84 | 0.69 |
| Celgosivir + Peg-IFN-α2a + Ribavirin | 25:40:6 | 0.58 | 0.49 | 0.42 |
| Celgosivir + Peg-IFN-α2a + Ribavirin | 25:20:3 | 0.79 | 0.76 | 0.74 |
| Celgosivir + Peg-IFN-α2b + NM-107 | 25:40:5 | 0.61 | 0.46 | 0.36 |
| Celgosivir + Peg-IFN-α2b + NM-107 | 25:20:2.5 | 0.64 | 0.58 | 0.54 |
| Celgosivir + Peg-IFN-α2b + Ribavirin | 25:40:6 | 0.67 | 0.58 | 0.51 |
| Celgosivir + Peg-IFN-α2b + Ribavirin | 25:20:3 | 0.83 | 0.84 | 0.86 |
| Celgosivir + Ribavirin + IFN-α2b | 25:3:30 | 0.84 | 0.58 | 0.41 |
| Celgosivir + Ribavirin + IFN-α2b | 25:6:60 | 0.68 | 0.53 | 0.43 |
| Castanospermine + Peg-IFN-α2a + NM-107 | 300:100:5 | 0.74 | 0.65 | 0.57 |

The triple combinations having celgosivir and NM-107 and various interferons (interferon-α-con-1, interferon-α-n3, interferon-α2b, Peg-interferon-α2a, Peg-interferon-α2b and interferon-λ1) all showed moderate to good synergistic activity at all ratios tested. The combination of celgosivir and NM-107 with interferon-ω showed good synergy (25:600:2.5) or moderate synergy (25:1200:5) depending on the ratio. Similarly, the triple combination of castanospermine, NM-107 and Peg-interferon-α2a showed good synergy. In all, the triple combinations showed surprisingly synergistic interactions against Flaviviridae infection.

TABLE 11
Superiority of Triple Compared to Double Combination Indexes (CI)

| Compound | Ratio | CI (EC₅₀) | CI (EC₇₅) | CI (EC₉₀) |
|---|---|---|---|---|
| Celgosivir + IFN-α-n3 | 25:80 | 0.82 | 0.72 | 0.64 |
| Celgosivir + NM-107 | 25:5 | 1.14 | 1.05 | 0.97 |
| Celgosivir + IFN-α-n3 + NM-107 | 25:80:5 | 0.72 | 0.67 | 0.64 |
| Celgosivir + Peg-IFN-α2a | 25:20 | 0.95 | 0.94 | 0.93 |
| Celgosivir + Peg-IFN-α2a + Ribavirin | 25:20:3 | 0.79 | 0.76 | 0.74 |
| Celgosivir + NM-107 | 20:2.22 | 0.81 | 0.83 | 0.86 |
| Celgosivir + NM-107 + IFN-α2b | 20:2.22:20 | 0.71 | 0.70 | 0.70 |
| Celgosivir + NM-107 + IFN-α2b | 25:5:30 | 0.96 | 0.78 | 0.65 |
| Celgosivir + Ribavirin | 25:6 | 1.00 | 1.00 | 1.06 |
| Celgosivir + Ribavirin + IFN-α2b | 25:6:60 | 0.68 | 0.53 | 0.43 |

In addition, the triple combinations having celgosivir, an interferon and a viral replication inhibitor (e.g., ribavirin or NM-107) generally showed better synergistic activity than the related double combinations of celgosivir and interferon or celgosivir and a viral replication inhibitor (see Table 11).

### Example 8

### Dose Effect of Interferon-α2b and/or Ribavirin on Castanospermine or Celgosivir Potency

The inhibition of cytopathic effect (CPE) assay of Example 2 was used to analyze the interaction of celgosivir or castanospermine combined with interferon-α2b in presence of increasing concentrations of ribavirin (from about 1.1 to 3.3 μM). Each double or triple combination was performed twice. The combination efficacy data were analyzed using the MacSynergy™ II software program as described herein. The EC₅₀ for each of celgosivir and castanospermine was calculated from the triple combination studies described in Example 6.

The EC₅₀ for each of celgosivir and castanospermine showed a dose-dependent decrease with increasing concentrations of interferon-α2b (see Tables 12 and 13, respectivelyError! Reference source not found.).

TABLE 12
Effect of Interferon and/or Ribavirin on the EC₅₀ of Celgosivir.

| | Average Celgosivir EC₅₀ (μM)^{†} | | | | |
|---|---|---|---|---|---|
| | Interferon-α2b (IU/mL) | | | | |
| Ribavirin (μM) | 0 | 0.7 | 2.2 | 6.7 | 20 |
| 0 | 6.5 ± 1.3 | 4.8 ± 0.7 | 3.9 ± 0.9 | 1.6 ± 0.6 | <0.4 |
| 0.37 | NT* | 4.3 ± 1.0 | 2.7 ± 1.3 | 1.0 ± 0.7 | <0.3 |
| 1.1 | NT | 2.9 ± 0.9 | 2.1 ± 0.2 | <0.9 | <0.3 |
| 3.3 | NT | <1.0 | <0.3 | <0.3 | <0.3 |

*NT means “Not tested”
^{†}Celgosivir concentrations tested were 20-0.3 μM

As the concentration of interferon-α2b increases from 0 to 20 IU/mL, the EC₅₀ of celgosivir decreased from about 6.5 to less than 0.4 μM (see Table 12Error! Reference source not found.). This reduction in EC₅₀ was even more pronounced when increasing concentrations of ribavirin were added to the double combination of celgosivir and interferon-α2b. Thus, the amount of celgosivir used in the combination treatments can be reduced due to the presence of ribavirin and/or interferon.

TABLE 13
Effect of Interferon and/or Ribavirin on the EC₅₀ of Castanospermine.

| | Average Castanospermine EC₅₀ (μM)^{†} | | | | |
|---|---|---|---|---|---|
| Ribavirin | Interferon-α2b (IU/mL) | | | | |
| (μM) | 0 | 0.7 | 2.2 | 6.7 | 20 |
| 0 | 52.2 ± 9.5 | 39.0 ± 13.4 | 18.9 ± 2.1 | 11.9 ± 2.5 | <1.3 |
| 0.12 | NT | 34.3 ± 3.4 | 23.4 ± 2.4 | 9.7 ± 4.8 | <1.4 |
| 0.37 | NT | 27.7 ± 6.3 | 19.7 ± 5.2 | 6.5 ± 7.5 | <1.5 |
| 1.1 | NT | 14.0 ± 4.5 | 6.9 ± 1 | <1.2 | <1.2 |
| 3.3 | NT | <1.2 | <1.2 | <1.2 | <1.2 |

^{†}Castanospermine concentrations tested were 100-1.2 μM.

As the concentration of interferon-α2b increases from 0 to 20 IU/mL, the EC₅₀ of castanospermine decreased from about 52 μM to less than about 1.3 μM (see Table 13Error! Reference source not found.). This reduction in EC₅₀ was even more pronounced when increasing concentrations of ribavirin were added to the double combination of castanospermine and interferon-α2b. Thus, the amount of castanospermine used in the combination treatments can be reduced due to the presence of ribavirin and/or interferon.

### Example 9

### Dose Effect of Castanospermine or Celgosivir on Interferon-α2b Potency

The inhibition of cytopathic effect (CPE) assay of Example 2 was used to analyze the interaction of celgosivir or castanospermine combined with interferon-α2b in presence of increasing concentrations of ribavirin (from about 1.1 to 3.3 μM). Each double or triple combination was performed twice. The combination efficacy data were analyzed using the MacSynergy™ software program as described herein. The EC₅₀ for interferon-α2b was calculated from the triple combination studies described in Example 6.

The EC₅₀ of interferon-α2b showed a dose-dependent decrease with increasing concentrations of castanospermine or celgosivir (Table 14Error! Reference source not found.).

TABLE 14
Dose-Effects of Combinations on IFN-α2b Potency

| | | | | | |
|---|---|---|---|---|---|
| Celgosivir added (μM) | 0 | 0.25 | 0.74 | 2.2 | 6.7 |
| Average Interferon-α2b EC₅₀ (IU/mL)^{†} | 19 | 21 | 14 | 5 | <1 |
| Castanospermine added (μM) | 0 | 1.2 | 3.7 | 11 | 33 |
| Average Interferon-α2b EC50 (IU/mL) | 16 | 18 | 13 | 7 | 1 |

^{†}Interferon-α2b concentrations tested were 60-0.7 IU/mL

As the concentration of castanospermine or celgosivir is increased, the EC₅₀ Of interferon-α2b decreased from about 20 IU/mL to less than about 1 IU/mL (see Table 14Error! Reference source not found.). This reduction in EC₅₀ was even more pronounced when increasing concentrations of ribavirin were added to the double combination of castanospermine or celgosivir and interferon-α2b (data not shown). Thus, the amount of interferon used in the combination treatments can be reduced due to the presence of castanospermine or celgosivir and/or ribavirin.

### Example 10

### Dose-Reduction Indexes of the Double and Triple Fixed Ratio Combinations

The combination results described in Examples 4, 5, and 7 were used to determine the Dose-Reduction Index (DRI) as described by Chou and Chou (Pharmacologist 30:231, 1988) as calculated by the Calcusyn™ 2 software (Biosoft). The DRI is a measure of how much the dose of each drug in a synergistic combination may be reduced at a given effect level compared with the doses for each drug acting alone. The DRI is important in clinical situations in which dose-reduction leads to a therapeutic regiment having a reduced toxicity profile for a patient and at the same time retaining therapeutic efficacy. Table 15 shows the DRIs of the double combinations at the EC₅₀ and Table 16 the DRIs at the EC₉₀. The DRIs for the triple combinations showing superiority to the corresponding double combinations are in bold and underlined.

TABLE 15
DRI (EC₅₀) of the Double and Triple Celgosivir or Castanospermine Combinations

| Combination | DRI (EC₅₀) | | |
|---|---|---|---|
| | Celgosivir | Peg-IFN-α2a | NM-107 |
| CEL + Peg-IFN-α2a (25:20) | 1.3 | 4.9 | |
| CEL + Peg-IFN-α2a + NM-107 (25:20:5) | 2.0 | 11.2 | 3.4 |
| CEL + Peg-IFN-α2a (25:40) | 1.7 | 4.8 | |
| CEL + Peg-IFN-α2a + NM-107 (25:40:10) | 3.1 | 8.8 | 2.6 |
| CEL + Peg-IFN-α2a (25:100) | 2.8 | 1.7 | |
| CEL + Peg-IFN-α2a + NM-107 (25:100:5) | 3.0 | 1.8 | 6.8 |
| | Celgosivir | Peg-IFN-α2a | RBV |
| CEL + Peg-IFN-α2a (25:40) | 2.2 | 9.9 | |
| CEL + Peg-IFN-α2a + RBV (25:40:6) | 2.4 | 11.0 | 12.0 |
| | Celgosivir | NM-107 | IFN-α2b |
| CEL + NM-107 (25:5) | 1.5 | 2.0 | |
| CEL + NM-107 + IFN-α2b (25:5:30) | 2.2 | 2.9 | 6.2 |
| CEL + NM-107 (25:10) | 2.5 | 1.6 | |
| CEL + NM-107 + IFN-α2b (25:10:60) | 3.4 | 2.2 | 4.8 |
| CEL + NM 107 (20:6.67) | 3.2 | 4.2 | |
| CEL + NM 107 + IFN-α2b (20:6.67:20) | 3.1 | 4.1 | 21.0 |
| CEL + NM 107 (‘20:2.2) | 1.5 | 6.1 | |
| CEL + NM 107 + IFN-α2b (20:2.2:20) | 2.0 | 7.9 | 13.3 |
| | Celgosivir | Peg-IFN-α2b | NM-107 |
| CEL + Peg-IFN-α2b (25:20) | 2.1 | 7.4 | |
| CEL + Peg-IFN-α2b + NM-107 (25:20:2.5) | 2.9 | 10.0 | 2.5 |
| CEL + Peg-IFN-α2b (25:40) | 2.9 | 4.9 | |
| CEL + Peg-IFN-α2b + NM-107 (25:40:5) | 4.5 | 7.7 | 2.0 |
| | Celgosivir | Peg-IFN-α2b | RBV |
| CEL + Peg-IFN-α2b (25:40) | 2.1 | 5.0 | |
| CEL + Peg-IFN-α2b + RBV (25:40:6) | 2.4 | 5.9 | 10.9 |
| CEL + Peg-IFN-α2b (25:20) | 1.7 | 8.4 | |
| CEL + Peg-IFN-α2b + RBV (25:20:3) | 1.6 | 7.7 | 14.2 |
| | Celgosivir | IFN-α-n3 | NM-107 |
| CEL + IFN-α-n3 (25:80) | 2.1 | 3.0 | |
| CEL + IFN-α-n3 + NM-107 (25:80:5) | 3.4 | 5.0 | 4.4 |
| | Celgosivir | IFN-ω | NM-107 |
| CEL + IFN-ω (25:60:2.5) | 2.7 | 5.7 | |
| CEL + IFN-ω + NM-107 (25:60:2.5) | 3.1 | 6.5 | 6.8 |
| CEL + IFN-ω (25:120) | 2.8 | 2.9 | |
| CEL + IFN-ω + NM-107 (25:120:5) | 4.1 | 4.2 | 4.4 |
| | Celgosivir | IFN-λ1 | NM-107 |
| CEL + IFN-λ1 (25:400) | 2.0 | 9.4 | |
| CEL + IFN-λ1 + NM-107 (25:400:2.5) | 2.6 | 12.3 | 6.1 |
| CEL + IFN-λ1 (25:800) | 2.0 | 4.8 | |
| CEL + IFN-λ1 + NM-107 (25:800:5) | 3.5 | 8.4 | 4.2 |
| | Celgosivir | RBV | IFN-α2b |
| CEL + RBV (25:3) | 1.2 | 8.4 | |
| CEL + RBV + IFN-α2b (25:3:30) | 1.7 | 11.3 | 6.8 |
| CEL + RBV (25:6) | 1.3 | 4.4 | |
| CEL + RBV + IFN-α2b (25:6:60) | 2.6 | 8.9 | 5.4 |
| | Celgosivir | IFN-α2b | RBV |
| CEL + IFN-α2b (25:60) | 2.3 | 4.4 | |
| CEL + IFN-α2b + RBV (25:60:6) | 3.1 | 6.1 | 8.5 |
| CEL + IFN-α2b (25:30) | 2.2 | 8.7 | |
| CEL + IFN-α2b + RBV (25:30:3) | 2.2 | 8.7 | 12.2 |
| | Celgosivir | IFN-α-Con-1 | NM-107 |
| CEL + IFN-αCon-1 (25:400) | 4.0 | 2.5 | |
| CEL + IFN-αCon-1 + NM-107 (25:400:5) | 5.3 | 3.3 | 2.1 |
| CEL + IFN-αCon-1 (25:200) | 3.3 | 4.1 | |
| CEL + IFN-αCon-1 + NM-107 (25:200:2.5) | 3.7 | 4.6 | 3.0 |
| | Castanospermine | Peg-IFN-α2a | NM-107 |
| CAST + Peg-IFN-α2a (300:5) | 3.5 | 3.6 | |
| CAST + Peg-IFN-α2a + NM-107 (300:100:5) | 3.3 | 3.4 | 7.3 |

TABLE 16
DRI (EC₉₀) of the Double and Triple Celgosivir or Castanospermine Combinations

| | |
|---|---|
| Combination | DRI (EC₉₀) |

| | Celgosivir | Peg-IFN-α2a | NM-107 |
|---|---|---|---|
| CEL + Peg-IFN-α2a (25:20) | 1.3 | 6.6 | |
| CEL + Peg-IFN-α2a + NM-107 (25:20:5) | 2.5 | 20.4 | 3.7 |
| CEL + Peg-IFN-α2a (25:40) | 1.9 | 7.6 | |
| CEL + Peg-IFN-α2a + NM-107 (25:40:10) | 4.7 | 19.0 | 3.5 |
| CEL + Peg-IFN-α2a (25:100) | 4.8 | 3.6 | |
| CEL + Peg-IFN-α2a + NM-107 (25:100:5) | 4.3 | 3.2 | 7.0 |
| | Celgosivir | Peg-IFN-α2a | RBV |
| CEL + Peg-IFN-α2a (25:40) | 3.4 | 32.2 | |
| CEL + Peg-IFN-α2a + RBV (25:40:6) | 3.0 | 28.3 | 21.1 |
| | CEL | NM-107 | IFN-α2b |
| CEL + NM-107 (25:5) | 2.0 | 2.1 | |
| CEL + NM-107 + IFN-α2b (25:5:30) | 3.4 | 3.6 | 14.0 |
| CEL + NM-107 (25:10) | 4.0 | 2.1 | |
| CEL + NM-107 + IFN-α2b (25:10:60) | 6.0 | 3.1 | 12.3 |
| CEL + NM 107 (20:6.67) | 2.8 | 3.8 | |
| CEL + NM 107 + IFN-α2b (20:6.67:20) | 4.1 | 5.5 | 44.8 |
| CEL + NM 107 (20:2.2) | 1.5 | 5.9 | |
| CEL + NM 107 + IFN-α2b (20:2.2:20) | 1.9 | 7.7 | 21.2 |
| | CEL | Peg-IFN-α2b | NM-107 |
| CEL + Peg-IFN-α2b (25:20) | 2.7 | 17.5 | |
| CEL + Peg-IFN-α2b + NM-107 (25:20:2.5) | 3.9 | 25.0 | 2.0 |
| CEL + Peg-IFN-α2b (25:40) | 5.0 | 16.0 | |
| CEL + Peg-IFN-α2b + NM-107 (25:40:5) | 9.0 | 28.8 | 2.3 |
| | CEL | Peg-IFN-α2b | RBV |
| CEL + Peg-IFN-α2b (25:40) | 2.4 | 12.9 | |
| CEL + Peg-IFN-α2b + RBV (25:40:6) | 2.6 | 14.4 | 17.1 |
| CEL + Peg-IFN-α2b (25:20) | 2.1 | 23.2 | |
| CEL + Peg-IFN-α2b + RBV (25:20:3) | 1.4 | 14.9 | 17.7 |
| | CEL | IFN-α-n3 | NM-107 |
| CEL + IFN-α-n3 (25:80) | 2.1 | 6.0 | |
| CEL + IFN-α-n3 + NM-107 (25:80:5) | 3.5 | 10.0 | 3.9 |
| | CEL | IFN-ω | NM-107 |
| CEL + IFN-ω (25:60:2.5) | 2.2 | 7.9 | |
| CEL + IFN-ω + NM-107 (25:60:2.5) | 3.3 | 11.5 | 6.4 |
| CEL + IFN-ω (25:120) | 2.6 | 4.5 | |
| CEL + IFN-ω + NM-107 (25:120:5) | 3.6 | 6.2 | 3.4 |
| | CEL | IFN-λ1 | NM-107 |
| CEL + IFN-λ1 (25:400) | 1.9 | 23.7 | |
| CEL + IFN-λ1 + NM-107 (25:400:2.5) | 2.5 | 31.3 | 5.2 |
| CEL + IFN-λ1 (25:800) | 2.0 | 12.7 | |
| CEL + IFN-λ1 + NM-107 (25:800:5) | 3.7 | 23.2 | 3.9 |
| | CEL | RBV | IFN-α2b |
| CEL + RBV (25:3) | 1.4 | 35.3 | |
| CEL + RBV + IFN-α2b (25:3:30) | 1.7 | 11.3 | 6.8 |
| CEL + RBV (25:6) | 1.0 | 12.6 | |
| CEL + RBV + IFN-α2b (25:6:60) | 3.3 | 40.2 | 10.5 |
| | CEL | IFN-α2b | RBV |
| CEL + IFN-α2b (25:60) | 2.4 | 3.9 | |
| CEL + IFN-α2b + RBV (25:60:6) | 3.1 | 5.1 | 17.5 |
| CEL + IFN-α2b (25:30) | 2.4 | 7.6 | |
| CEL + IFN-α2b + RBV (25:30:3) | 2.3 | 7.6 | 26.2 |
| | CEL | IFN-αcon-1 | NM-107 |
| CEL + IFN-αcon-1 (25:400) | 8.5 | 3.2 | |
| CEL + IFN-α-con-1 + NM-107 (25:400:5) | 13.2 | 4.9 | 2.0 |
| CEL + IFN-αcon-1 (25:200) | 7.1 | 5.3 | |
| CEL + IFN-α-con-1 + NM-107 (25:200:2.5) | 8.3 | 6.2 | 2.5 |
| | CAST | Peg-IFN-α2a | NM-107 |
| CAST + Peg-IFN-α2a (300:5) | 4.5 | 4.9 | |
| CAST + Peg-IFN-α2a + NM-107 (300:100:5) | 4.4 | 4.7 | 7.6 |

The triple combinations generally not only show an unexpected synergistic interaction, but also show a potential dose reduction index for the component compounds of the triple combinations as compared with the double combinations.

### Example 11

### Reduction of Drug Cytotoxicity in Double and Triple Combinations Checkerboard Approach

The cytotoxicity of test compound combinations was determined in parallel to the efficacy assessments described in Examples 3 and 6, and analyzed using the MacSynergy™ II software program as described herein. In this case, a greater negative percent volume (antagonism) is indicative of the combination having a reduced cytotoxic activity. A value of less than −25 μM(IU/mL) % or μM² is considered a significant antagonistic effect (i.e., a significant decrease in cytotoxicity), while a value between −25 and 0 μM(IU/mL) or μM² is considered a non-significant change in cytotoxicity.

TABLE 17
Cytotoxicity Synergy and Antagonism Volumes of Double
Combinations

| | Cytotoxicity (95% CI) | |
|---|---|---|
| Double Combinations | Synergy | Antagonism |
| Celgosivir + IFN α2b (μM(IU/mL)%) | 0 | −117 ± 16 |
| Celgosivir + Ribavirin (μM2 %) | 0 | −101 ± 15 |
| Celgosivir + Amantadine (μM² %) | 19 | −117 |
| Celgosivir + 2-BAIP (μM² %) | 1.6 | −27 |
| Celgosivir + NB-DNJ (μM² %) | 27 | −2.1 |
| Castanospermine + IFN-α2b (μM(IU/mL)%) | 0 | −63 ± 10 |
| Castanospermine + Ribavirin (μM² %) | 0 | −46 + 13 |
| Castanospermine + Amantadine (μM² %) | 1.3 | −40.1 |
| Castanospermine + 2-BAIP (μM² %) | 0 | −26.3 |
| Castanospermine + NB-DNJ (μM² %) | 8.1 | −1.7 |
| Ribavirin + IFN-α2b (μM(IU/mL)%) | 6 ± 1 | −83 ± 18 |

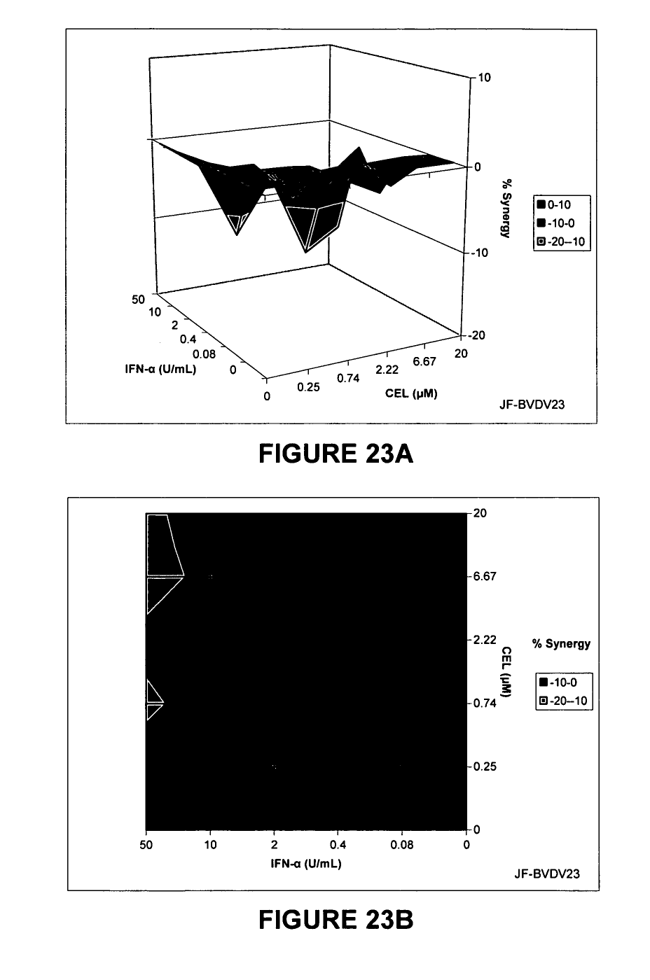

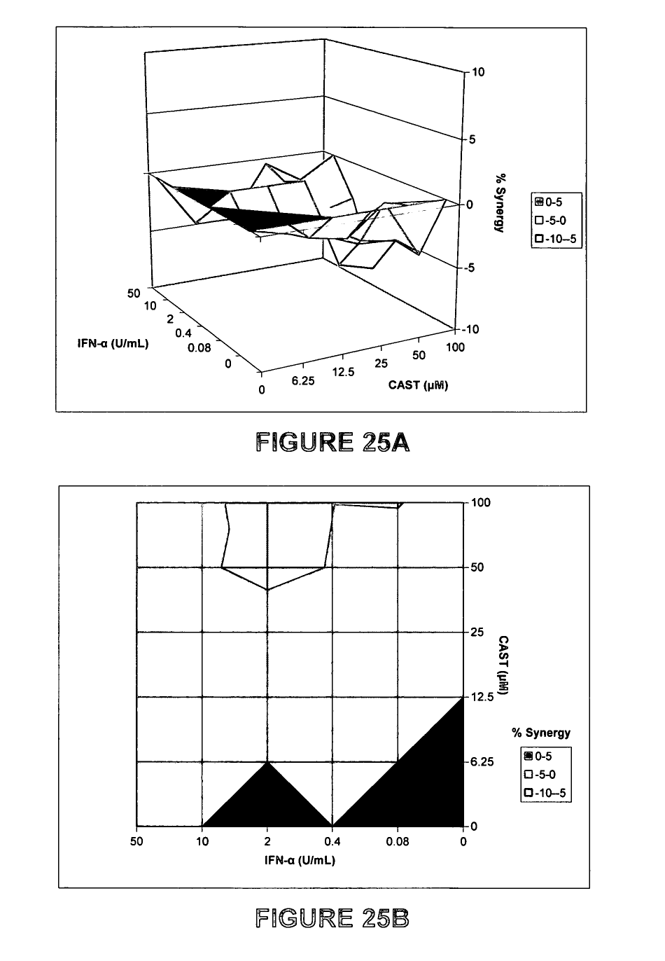

The combinations of celgosivir with IFN-α2b and castanospermine with IFN-α2b showed strong and moderate antagonistic effects on cytotoxicity, respectively, in uninfected MDBK cells, while no increase in cytotoxicity (i.e., synergistic effects) were found,(see Table 17). For the combination of celgosivir with IFN-α2b, antagonistic troughs were located at celgosivir concentrations greater than or equal to about 0.7 μM, and at interferon-α2b concentrations of greater than about 10 IU/mL (see FIG. 23). For the combination of castanospermine with interferon-α2b, antagonistic troughs were located at castanospermine concentrations of between about 50 and 100 μM, and interferon-α2b concentrations of greater than about 0.4 IU/mL (see FIG. 25).

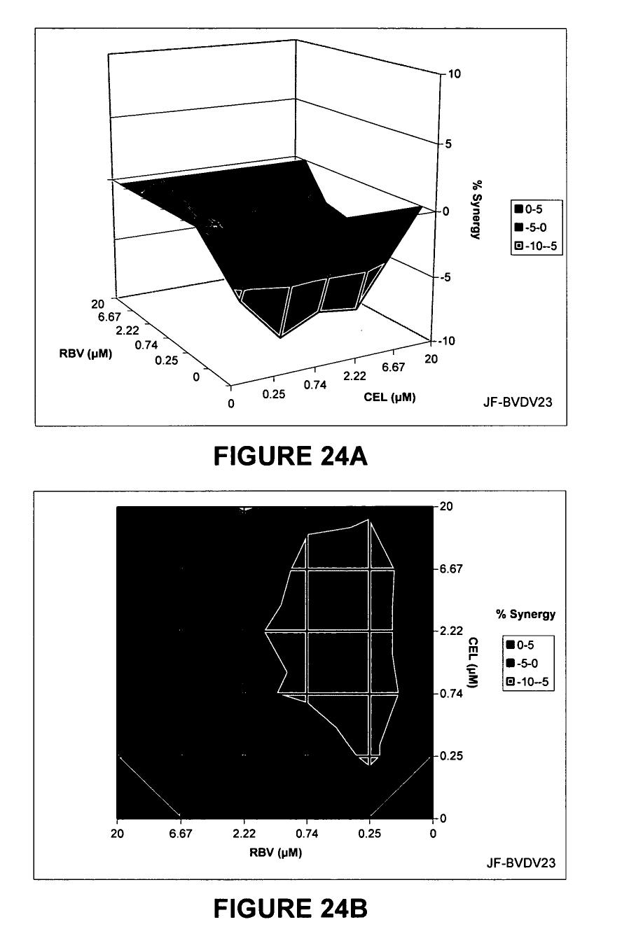

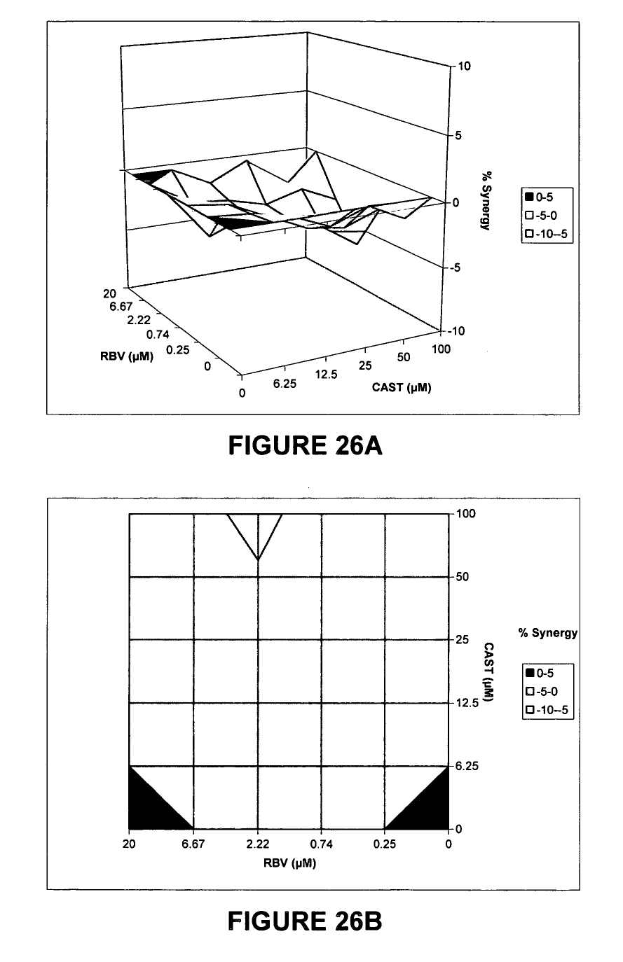

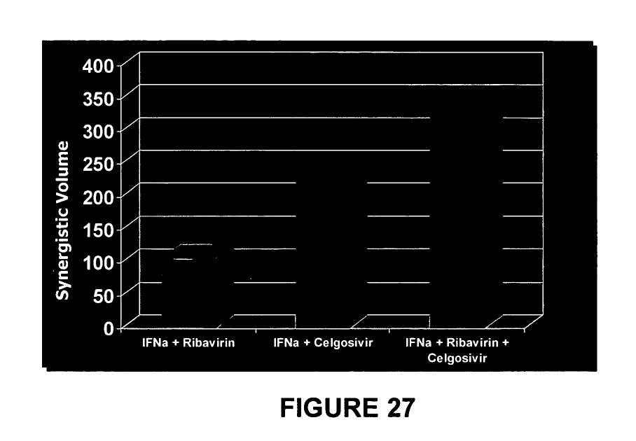

The combination of celgosivir with ribavirin showed strong antagonistic effects on cytotoxicity (−101 μM²%) in uninfected MDBK cells, while no synergistic (increase in) cytotoxic effects were observed (see Table 17). Antagonistic troughs were located at celgosivir concentrations of between about 0.25 to 20 μM, and at ribavirin concentrations of between about 0.25 and 2.2 μM (see FIG. 24). The combination of castanospermine with ribavirin showed moderate antagonistic effects on cytotoxicity (−46 μM²%) in uninfected MDBK cells, while no synergistic cytotoxic effects were observed (see Table 17). Antagonistic troughs were located at castanospermine concentrations of greater than about 20 μM, and at ribavirin concentrations of approximately 3 μM (see FIG. 26).

The cytotoxicity of castanospermine or celgosivir in combination with amantadine, 2-BAIP, or NB-DNJ was determined in uninfected MDBK cells and the cytotoxicity volumes for these double combinations were generally additive (i.e., volumes of synergy between 0 and 25 μM²%) or moderately antagonistic, indicating that addition of castanospermine to amantadine or 2-BAIP may reduce the expected toxicities of the latter compounds.

The standard HCV combination treatment of interferon-α2b with ribavirin showed a moderate antagonistic effect on cytotoxicity (see Table 17). Antagonism was quite uniform throughout the concentration ranges of these two antivirals with no concentration region experiencing significantly higher antagonism than any other areas (data not shown). The maximum percent antagonism reached was about −10%. The cytotoxic volumes for the combinations were generally antagonistic, indicating that the combinations had no significant impact on the cytotoxicity of the individual compounds, but this antagonism may indicate that the combinations can reduce the individual cytotoxicities of the test compounds. As set forth above, a dose-dependent reduction in EC₅₀ of castanospermine (up to about 52-fold) and celgosivir (up to about 26-fold) was observed upon the addition of increasing concentrations of interferon-α2b (see Examples 8 and 9). This decrease in EC₅₀ was more pronounced with the addition of increasing concentrations of ribavirin. Fortunately the combinations did not increase the cytotoxicity of either interferon-α2b or ribavirin. These data indicate that the combination of celgosivir or castanospermine with interferon-α2b and/or ribavirin could be beneficial for devising less toxic treatment regimes for HCV-infected patients while improving therapeutic effects.

TABLE 18
Cytotoxicity Antagonism Volume of Triple Combination Treatment Studies (at
Various Ribavirin Concentrations)

| Treatment | Antagonism Volume (μM(IU/mL)%)* | | | | |
|---|---|---|---|---|---|
| Ribavirin (μM) | 0 | 0.12 | 0.37 | 1.1 | 3.3 |
| Celgosivir + Interferon-α2b | −65 ± 59 | −66 ± 38 | −97 ± 41 | −29 ± 5 | −8 ± 8 |
| Castanospermine + Interferon-α2b | −154 ± 72 | −90 ± 57 | −77 ± 99 | −23 ± 11 | −77 ± 108 |

*The average synergistic volume is at a confidence level of 95%.

The cytotoxic volumes of the triple combination celgosivir, interferon-α2b and ribavirin were strongly antagonistic (values of less than about −100 μM(IU/mL) %) when up to 1.1 μM ribavirin was added to the celgosivir/interferon-α2b combination (see Table 18). No significant synergy in cytotoxicity was observed in the triple combination of celgosivir, interferon-α2b and ribavirin. The cytotoxic antagonistic volumes of the castanospermine, interferon-α2b and ribavirin combination were minor to moderate (see Table 18), while the cytotoxic synergism volumes were not significant to minor (see Table 19). Thus, the triple combinations can provide advantages for devising dosing regimes for treating HCV-infected patients.

TABLE 19
Cytotoxicity Synergy Volume of Triple Combinations (at Various
Ribavirin Concentrations)

| | Average Synergistic | | | | |
|---|---|---|---|---|---|
| Treatment | Volume (μM(IU/mL)%)* | | | | |
| Ribavirin (μM) | 0 | 0.12 | 0.37 | 1.1 | 3.3 |
| Celgosivir + Interferon-α2b | 0 | 0 | 0 | 3 ± 1 | 8 ± 8 |
| Castanospermine + Interferon-α2b | 0 | 0 | 0 | 16 ± 3 | 29 ± 41 |

*The average synergistic volume is at a confidence level of 95%.

### Example 12

### Pharmacokinetics of Castanospermine or Celgosivir in the Presence of an Anti-Diarrheal Agent

The purpose of this study was to evaluate the effect of an anti-diarrheal agent on the pharmacokinetics (PK) of orally administered celgosivir. In addition, the effect of anti-diarrheal agents on celgosivir PK was investigated. The pharmacokinetics of celgosivir was assessed by following the plasma profile of celgosivir's primary metabolite, castanospermine. By way of background, orally administered celgosivir, although well tolerated in humans, does produce side effects in the gastrointestinal tract, including flatulence and mild to moderate diarrhea. Loperamide hydrochloride, an anti-motility agent that is an active ingredient found in some over-the-counter medications used for symptomatic relief of acute and chronic diarrhea, was investigated for its effect on the PK of orally administered celgosivir.

Male Sprague-Dawley rats (Crl;CD) were obtained from Charles River Laboratories (Montreal, Canada). The rats weighed from about 200 g to about 400 g, and dose levels were adjusted according to the weight of each animal. Dose levels of celgosivir and loperamide were based on human doses adjusted to total body surface area. A first group of six rats (Normal Control) were administered a single oral dose of celgosivir at 35 mg/kg. A second group of six rats (Loperamide-treated) were administered a single oral dose of loperamide at 0.35 mg/kg, then ten minutes later each animal was given a single oral dose of celgosivir at 35 mg/kg. A third group of six rats (diarrhea-induced) were fasted for approximately 18 hours with free access to water. Castor oil was then administered as a single oral dose of 5 mL/kg, then immediately given free access to food. One hour after castor oil administration, each rat was administered a single oral dose of celgosivir at 35 mg/kg. A fourth group of six rats (Fasted Controls) were fasted for approximately 18 hours with free access to water, and then allowed free access to food for approximately 30 minutes prior to administration of a single oral dose of celgosivir at 35 mg/kg.

At various time-points after celgosivir administration, blood samples were withdrawn from animals via the tail vein. Plasma samples were generated and stored frozen until analyzed. Plasma samples were analyzed for castanospermine, the major metabolite of celgosivir, using LC/MS. Briefly, samples were extracted using solid phase extraction (SPE) followed by separation by reversed-phase HPLC and MS detection in electrospray positive mode. The range of the bio-analytical assay was from about 0.1 to about 50 μg/mL. Celgosivir pharmacokinetics was assessed by following the plasma concentration of its primary metabolite, castanospermine. Pharmacokinetic parameters were calculated according to a two-compartment model with bio-exponential decay using the method of residuals. Pharmacokinetic parameters were compared using an unpaired student t test with 95% confidence intervals (p=0.01).

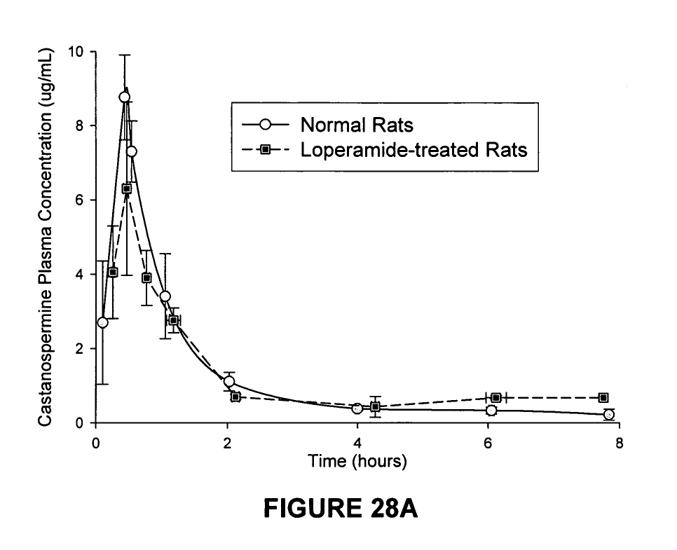

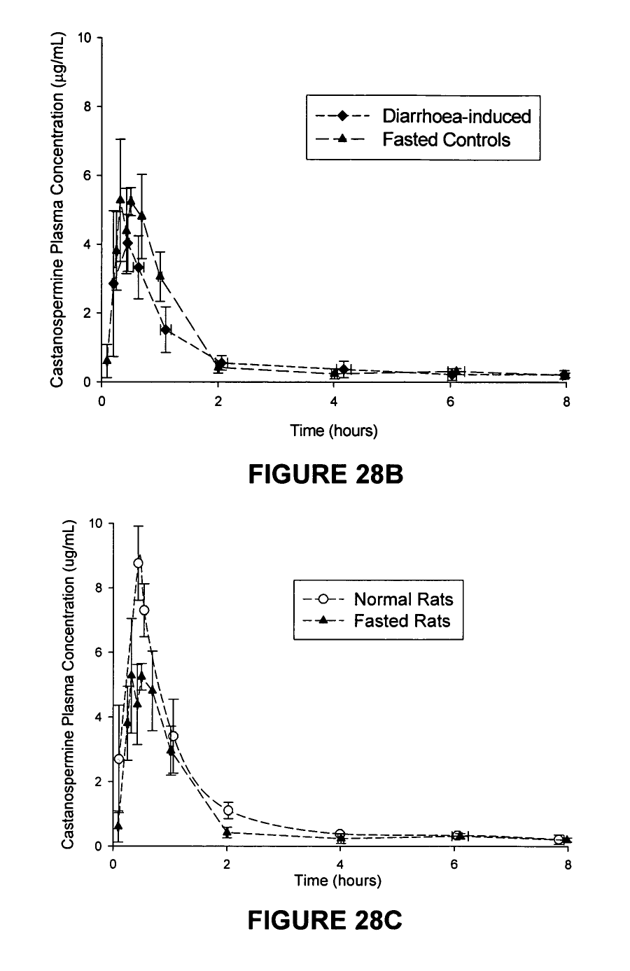

Oral administration of a 35 mg/kg dose of celgosivir to normal rats (Normal Control) resulted in castanospermine Cₘₐₓ, tₘₐₓ and AUC values of 8.8 μg/mL, 0.44 hour and 10.5 μg·hour/mL, respectively. Comparable results were obtained in animals that had been pre-administered a 0.35 mg/kg dose of lopeeramide (Loperamide-treated) (see FIG. 28A and Table 20).

TABLE 20
Summary of Pharmacokinetic Parameters

| | | | | AUC |
|---|---|---|---|---|
| Treatment | | Cₘₐₓ | tₘₐₓ | (μg · hour/ |
| Group | Description | (μg/mL) | (hours) | mL) |
| 1 | Normal Control | 8.76 ± 1.15 | 0.44 + 0.01 | 10.5 |
| 2 | Loperamide-treated | 6.30 ± 2.33 | 0.47 ± 0.05 | 9.5 |
| 3 | Diarrhea-induced | 4.03 ± 0.83 | 0.44 ± 0.01 | 5.9 |
| 4 | Fasted Control | 5.28 ± 1.77 | 0.32 | 7.2 |

The effect of diarrhea on the PK of celgosivir was also investigated. When comparing the Normal control group to the Diarrhea-induced group, the castanospermine Cₘₐₓ and AUC values were reduced by 54% and 44%, respectively (Table 20). The difference in Cₘₐₓ was determined to be statistically significant. Induction of diarrhea with castor oil required overnight fasting (approximately 18 hours) followed by administration of castor oil with immediate access to food. To determine the effect of overnight fasting, a Fasted Control group was used, and castanospermine Cₘₐₓ and AUC values in these animals were somewhere in between those obtained for the Normal Control group and the Diarrhea-Induced group (FIGS. 28B and 28C, and Table 20). These results indicate that both fasting and diarrhea may reduce the Cmax and AUC for orally administered celgosivir.

Concomitant administration of an anti-diarrheal agent had no significant effect on the PK of celgosivir in normal rats and could be considered as a viable option for reducing gastrointestinal effects that may be associated with celgosivir treatment. Diarrhea-induced rats showed a reduction in castanospermine Cmax and AUC, so treatment with an anti-diarrheal agent might be useful in preventing lowered systemic drug exposure in patients experiencing diarrhea.

Those skilled in the art will recognize, or be able to ascertain, using no more than routine experimentation, many equivalents to the specific embodiments of the invention described herein. Such equivalents are intended to be encompassed by the following claims.

1. A combination of compounds comprising a glucosidase inhibitor, an agent that alters immune function, and an agent that alters replication of Flaviviridae.

2. The combination according to claim 1 wherein the glucosidase inhibitor has the following structural formula (I): 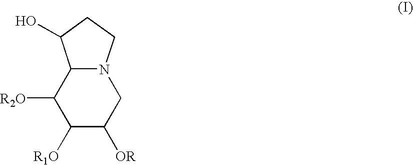
wherein R, R₁ and R₂ are independently hydrogen, C₁₋₁₄ alkanoyl, C₂₋₁₄ alkenoyl, cyclohexanecarbonyl, C₁₋₈ alkoxyacetyl, 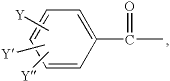
naphthalenecarbonyl optionally substituted by methyl or halogen; phenyl(C₂₋₆ alkanoyl) wherein the phenyl is optionally substituted by methyl or halogen; cinnamoyl; pyridinecarbonyl optionally substituted by methyl or halogen; dihydropyridine carbonyl optionally substituted by C₁₋₁₀ alkyl; thiophenecarbonyl optionally substituted by methyl or halogen; or furancarbonyl optionally substituted by methyl or halogen; Y is hydrogen, C₁₋₄ alkyl, C₁₋₄ alkoxy, halogen, trifluoromethyl, C₁₋₄ alkylsulphonyl, C₁₋₄ alkylmercapto, cyano or dimethylamino; Y′ is hydrogen, C₁₋₄ alkyl, C₁₋₄ alkoxy, halogen or it is combined with Y to give 3,4-methylenedioxy; Y″ is hydrogen, C₁₋₄ alkyl, C₁₋₄ alkoxy or halogen; or a pharmaceutically acceptable salt or derivative thereof;
and pharmaceutically acceptable salts thereof.

3. The combination according to claim 2 wherein the glucosidase inhibitor structural formula (I) has the following stereochemistry:

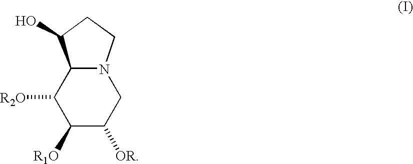

4. The combination according to claim 2 wherein R, R₁ and R₂ are each independently hydrogen, C₁₋₁₀ alkanoyl, C₂₋₁₀ alkenoyl, C₁₋₈ alkoxyacetyl; or 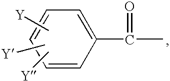
wherein Y is hydrogen, C₁₋₄ alkyl, C₁₋₄ alkoxy, halogen, trifluoromethyl, C₁₋₄ alkylsulphonyl, C₁₋₄ alkylmercapto, cyano or dimethylamino;
Y′ is hydrogen, C₁₋₄ alkyl, C₁₋₄ alkoxy, halogen or it is combined with Y to give 3,4-methylenedioxy;
Y″ is hydrogen, C₁₋₄ alkoxy or halogen; and
wherein at least one, but not more than two, of R, R₁ and R₂ is hydrogen.

5. The combination according to claim 2 wherein R, R_{1 and R}₂ are each independently hydrogen, C₁₋₈ alkanoyl, C₂₋₈ alkenoyl, C₁₋₈ alkoxy-acetyl, or a benzoyl optionally substituted with an alkyl or halogen; and
wherein at least one, but not more than two, of R, R₁ and R₂ is hydrogen.

6. The combination according to claim 2 wherein R, R₁ and R₂ are each independently hydrogen, C₁₋₈ alkanoyl, C₂₋₈ alkenoyl, C₁₋₈ alkoxy-acetyl, or a benzoyl optionally substituted with a methyl, bromo, chloro, or fluoro group; and
wherein at least one, but not more than two, of R, R₁ and R₂ is hydrogen.

7. The combination according to claim 2 wherein RI is a C₁₋₈ alkanoyl, C₂₋₁₀ alkenoyl, C₁₋₈ alkoxy-acetyl, or a benzoyl optionally substituted with an alkyl or halogen group.

8. The combination according to claim 2 wherein R₁ is a C₁₋₈ alkanoyl, C₂₋₈ alkenoyl, C₁₋₈ alkoxyacetyl, or a benzoyl optionally substituted with a methyl, bromo, chloro, or fluoro group.

9. The combination according to claim 2 wherein the glucosidase inhibitor is:
(a) [1S-(1α,6β,7α,8β,8aβ)]-octahydro-1,6,7,8-indolizinetetrol 6-benzoate;
(b) [1S-(1α,6β,7α,8β,8aβ)]-octahydro-1,6,7,8-indolizinetetrol 7-benzoate;
(c) [1S-(1α,6β,7α,8β,8aβ)]-octahydro-1,6,7,8-indolizinetetrol 6-(4-methylbenzoate);
(d) [1S-(1α,6β,7α,8β,8aβ)]-octahydro-1,6,7,8-indolizinetetrol 7-(4bromobenzoate);
(e) [1S-(1α,6β,7α,8β,8aβ)]-octahydro-1,6,7,8-indolizinetetrol 6,8-dibutanoate;
(f) [1S-(1α,6β,7α,8β,8aβ)]-octahydro-1,6,7,8-indolizinetetrol 6-butanoate;
(g) [1S-(1α,6β,7α,8β,8aβ)]-octahydro-1,6,7,8-indolizinetetrol 6-(2-furancarbonxylate);
(h) [1S-(1α,6β,7α,8β,8aβ)]-octahydro-1,6,7,8-indolizinetetrol 7-(2,4-dichlorobenzoate);
(i) [1S-(1α,6β,7α,8β,8aβ)]-octahydro-1,6,7,8-indolizinetetrol 6-(3-hexenoate);
(j) [1S-(1α,6β,7α,8β,8aβ)]-octahydro-1,6,7,8-indolizinetetrol 6-octanoate;
(k) [1S-(1α,6β,7α,8β,8aβ)]-octahydro-1,6,7,8-indolizinetetrol 6-pentanoate;
(l) an O-pivaloyl ester;
(m) a 2-ethyl-butyryl ester;
(n) a 3,3-dimethylbutyryl ester;
(o) a cyclopropanoyl ester;
(p) a 4-methoxybenzoate ester;
(q) a 2-aminobenzoate ester;
(r) castanospermine; or
(s) a mixture of at least two of (a)-(r).

10. The combination according to claim 2 wherein the glucosidase inhibitor is [1S-(1α,6β,7α,8β,8aβ)]-octahydro-1,6,7,8-indolizinetetrol 6-benzoate.

11. The combination according to claim 2 wherein the glucosidase inhibitor is [1S-(1α,6β,7α,8β,8aβ)]-octahydro-1,6,7,8-indolizinetetrol 6-butanoate.

12. The combination according to claim 2 wherein the glucosidase inhibitor is [1S-(1α,6β,7α,8β,8aβ)]-octahydro-1,6,7,8-indolizinetetrol 6-pentanoate.

13. The combination according to claim 2 wherein the glucosidase inhibitor is [1S-(1α,6β,7α,8β,8aβ)]-octahydro-1 ,6,7,8-indolizinetetrol 6-(2-furancarbonxylate).

14. The combination according to claim 1 wherein the agent that alters immune function is an interferon.

15. The combination according to claim 14 wherein the interferon is an interferon-α.

16. The combination according to claim 14 or claim 15 wherein the interferon-α is pegylated.

17. The combination according to claim 1 wherein the agent that alters viral replication is ribavirin.

18. The combination according to claim 1 wherein the agent that alters viral replication is viramidine.

19. The combination according to claim 1 wherein the agent that alters viral replication is a nucleoside analogue.

20. The combination according to claim 1 wherein the nucleoside analogue is NM283.

21. The combination according to claim 1 wherein the nucleoside analogue is NM107.

22. The combination according to claim 1 wherein the Flaviviridae is a member of the genus Flavivirus.

23. The combination according to claim 1 wherein the Flaviviridae is a member of the genus Pestivirus.

24. The combination of claim 4 wherein the Flavivirus is a Hepacivirus, wherein the Hepacivirus is Hepatitis C virus (HCV).

25. The combination according to claim 1 wherein the composition further comprises an anti-diarrheal agent.

26. The combination according to claim 1 wherein the combination fuirther comprises:
(a) a compound that inhibits infection of cells by Flaviviridae;
(b) a compound that inhibits the release of Flaviviridae RNA from the viral capsid or inhibits the fuiction of Flaviviridae gene products;
(c) a compound that alters symptoms of a Flaviviridae infection; or (d) a compound for treating Flaviviridae-associated infections.

27. The combination according to claim 26 wherein the Flaviviridae-associated infection is a hepatitis B viral (HBV) infection or a retroviral infection.

28. The combination according to claim 27 wherein the retroviral infection is a human immunodeficiency virus infection (HIV).

29. A method for treating a Flaviviridae infection comprising administering to a subject a combination of a glucosidase inhibitor, an agent that alters immune function, and an agent that alters replication of Flaviviridae.

30. The method according to claim 29 wherein the glucosidase inhibitor is according to any one of claims 2 to 13.

31. The method according to claim 29 wherein the agent that alters immune function is pegylated interferon-α.

32. The method according to claim 29 wherein the agent that alters replication of Flaviviridae is ribavirin or viramidine.

33. The method according to claim 29 wherein the agent that alters replication of Flaviviridae is NM283 or NM107.

34. The method according to claim 29 wherein the Flaviviridae is a Hepatitis C virus (HCV).

35. The method according to claim 29 further comprising administering an anti-diarrheal agent.

36. The method according to claim 29 wherein the glucosidase inhibitor is administered orally.

37. The method according to any one of claims 29, 32 and 33 wherein the agent that alters replication of Flaviviridae is administered orally.

38. The method according to claim 29 or claim 31 wherein the agent that alters immune function is administered by injection.

39. The method according to claim 29 or claim 31 wherein the injection is subcutaneous.

40. The method according to claim 29 wherein the subject is a human.
